(19) United States
(12) Patent Application Publication
(10) Pub. No.: US 2005/0202077 A1
(43) Pub. Date: Sep. 15, 2005

(54) Targeted delivery of RNA interference molecules

(75) Inventors: James D. Watson, St. Heliers (NZ); J. Greg Murison, Sandringham (NZ); Murray R. Grigor, Glendowie (NZ); Ilkka Jaakko Havukkala, Remuera (NZ); Grant Munro, Rothesay Bay (NZ); Nevin Abernethy, Meadowbank (NZ); Gill Webster, Orakei (NZ)

Correspondence Address: SPECKMAN LAW GROUP PLLC, 1501 WESTERN AVE, SEATTLE, WA, 98101, US

(73) Assignee: GENESIS RESEARCH AND DEVELOPMENT CORPORATION LTD., Parnell (NZ)

(21) Appl. No.: 11/063,391

(22) Filed: Feb. 22, 2005

Related U.S. Application Data
(60) Provisional application No. 60/546,434, filed on Feb. 20, 2004.

(51) Int. Cl. C12Q 1/68; A61K 9/127; A61K 48/00; C12N 15/86

(52) U.S. Cl. 424/450; 435/456; 435/6; 514/44

(57) ABSTRACT

Compositions for the treatment and/or prevention of IgE-mediated disorders in a mammal by means of RNA interference are provided, together with methods for the use of such compounds. The inventive compositions comprise a binding agent that specifically binds to a target internalizable antigen that is expressed on the surface of a target cell of interest and a genetic construct that is capable of expressing a small interfering nucleic acid molecule (siNA) that suppresses expression of a target gene within the target cell, whereby, after binding to the target antigen, the binding agent and genetic construct are internalized into the cell, and the genetic construct released.

### REFERENCE TO RELATED APPLICATIONS

This application claims priority to U.S. provisional patent application No. 60/546,434, filed Feb. 20, 2004.

### FIELD OF THE INVENTION

The present invention relates to the treatment of disorders by means of RNA interference (RNAi). More specifically, the present invention relates to the targeted delivery of small nucleic acid molecules that are capable of mediating RNAi against genes that are active in key pathways involved in disorders, such as immunoglobulin-ε (IgE)-mediated disorders.

### BACKGROUND OF THE INVENTION

There are many allergic disorders, including allergic rhinitis (e.g. hay fever), asthma, anaphylaxis, urticaria (hives), atopic dermatitis (eczema) and food allergies, that are mediated by the antibody class known as immunoglobin epsilon (IgE). Individuals who are severely atopic, or allergic, typically have elevated levels of serum IgE. This class of antibody is produced by a specific class of B cells that have become committed to the production of IgE during their development. Once these B cells are activated by antigen, they secrete IgE antibodies, which then circulate in the blood and lymph systems and bind to FcεR1 on mast cells and basophils.

The induction of IgE synthesis in B cells involves the interaction of antigen (in allergic responses often referred to as allergen) with antigen presenting cells (APC) and a class of helper T cells known as T_{H}2 cells. When a B cell expressing an IgE molecule on its cell surface binds specifically to an antigen, or allergen, and also to the APC and T_{H}2 cells, the B cell is activated to begin synthesizing and secreting large numbers of IgE molecules into the blood system.

Very little IgE is found in circulation as it is rapidly captured by the high affinity IgE receptor (FcεR1) found on the surface of mast cells in the tissue and circulating basophils. The ligation of the cell-bound IgE by allergen triggers the release of the mediators that give rise to the allergic response. These mediators, which include histamine, leukotrienes, prostaglandins and cytokines (including IL-4, IL-5, IL-6, TNF and GM-CSF), cause both a rapid response, referred to as immediate hypersensitivity, and a delayed response, referred to as a late phase reaction, which occurs 2-24 hours after mast cell or basophil activation. Immediate hypersensitivity is characterized by increased vascular permeability, vasodilation, bronchial and visceral smooth muscle contraction, and local inflammation. The late phase reaction is characterized by an inflammatory infiltrate of eosinophils, basophils, neutrophils and lymphocytes. Repeated bouts of this late phase reaction can cause tissue damage.

In the process of activating B cells, T_{H}2 cells can release cytokines such as GM-CSF and IL-5, which in turn are capable of activating eosinophils to release mediators, which also include histamine, leukotrienes, prostaglandins and cytokines, thereby increasing the allergic response. Allergic disorders may thus be thought of as T_{H}2-dependent disorders.

Asthma is a common disease, with a high prevalence in the developed world. Asthma is characterized by increased responsiveness of the tracheobronchial tree to a variety of stimuli, the primary physiological disturbance being reversible airflow limitation, which may be spontaneous or drug-related, and the pathological hallmark being inflammation of the airways. It has been established that most asthma is a form of immediate hypersensitivity. Clinically, asthma can be subdivided into extrinsic and intrinsic variants.

Extrinsic asthma has an identifiable precipitant, and can be thought of as being atopic, occupational and drug-induced. Atopic asthma is associated with the enhancement of a T_{H}2-type of immune response with the production of specific IgE. The airflow obstruction in extrinsic asthma is due to nonspecific bronchial hyperesponsiveness caused by inflammation of the airways. In atopic asthma, the immune response producing airway inflammation is brought about by the T_{H}2 class of T cells which secrete IL-4, IL-5 and IL-10. It has been shown that lymphocytes from the lungs of atopic asthmatics produce IL-4 and IL-5 when activated. Both IL-4 and IL-5 are cytokines of the T_{H}2 class and are required for the production of IgE and involvement of eosinophils in asthma. Intrinsic, or cryptogenic, asthma is reported to develop after upper respiratory tract infections, but can arise de novo in middle-aged or older people, in whom it is more difficult to treat than extrinsic asthma.

Asthma is ideally prevented by the avoidance of triggering allergens but this is not always possible, nor are triggering allergens always easily identified. The medical therapy of asthma is based on the use of corticosteroids and bronchodilator drugs to reduce inflammation and reverse airway obstruction. In chronic asthma, treatment with corticosteroids leads to unacceptable adverse side effects.

Another disorder with a similar immune abnormality to asthma is allergic rhinitis. Allergic rhinitis is a common disorder and is estimated to affect at least 10% of the population. Allergic rhinitis may be seasonal (hay fever) caused by allergy to pollen. Non-seasonal, or perennial, rhinitis is caused by allergy to antigens such as those from house dust mite or animal dander.

The abnormal immune response in allergic rhinitis is characterized by the excess production of IgE antibodies specific against the allergen. The inflammatory response occurs in the nasal mucosa rather than further down the airways as in asthma. Like asthma, local eosinophilia in the affected tissues is a major feature of allergic rhinitis. As a result of this inflammation, patients develop sneezing, nasal discharge and congestion. In more severe cases, the inflammation extends to the eyes (conjunctivitis), palate and the external ear. While it is not life threatening, allergic rhinitis may be very disabling, preventing normal activities and interfering with a person's ability to work. Current treatment involves the use of antihistamines, nasal decongestants and, as for asthma, sodium cromoglycate and corticosteroids.

Atopic dermatitis, also known as atopic eczema, is a chronic and recurrent pruritic inflammatory skin disease which usually occurs in families with an hereditary predisposition for various allergic disorders, such as allergic rhinitis and asthma. Atopic dermatitis is increasing in prevalence with up to 15% of the population having had atopic dermatitis during childhood. The main symptoms are dry skin and dermatitis (eczema) localized mainly in the face, neck and on the flexor sides and folds of the extremities, accompanied by severe itching. It typically starts within the first five years of life. In many patients this skin disease disappears during childhood but the symptoms can continue into adult life. Furthermore, 50% of patients develop asthma and approximately 75% develop allergic rhinitis. Atopic dermatitis is one of the commonest forms of dermatitis worldwide.

Allergens play an important role in atopic dermatitis. Approximately 80% of patients have IgE antibodies to a variety of food and inhaled allergens, with the majority of patients with severe atopic dermatitis having elevated levels of serum IgE, particularly if they also have other forms of atopic disease. In addition, circulating levels of blood eosinophils are often elevated. In atopic dermatitis, the dermis of skin lesions is infiltrated with macrophages, T cells and eosinophils, and in chronic lesions there are increased numbers of mast cells. Acute lesions have significantly more cells expressing the cytokines IL-4, IL-5 and IL-13, indicating preferential accumulation of the Th2 class of T cells. In addition, circulating T cells in atopic dermatitis patients produce more IL-4 and IL-5, compared to normal individuals. IL-4 is responsible for switching antibody production to the IgE isotype, the development of T_{H}2 cells and induction of adhesion molecules on endothelial cells that recruit eosinophils. IL-5 is important for the development and differentiation of eosinophils.

Allergic contact dermatitis is a common non-infectious inflammatory disorder of the skin. In contact dermatitis, immunological reactions cannot develop until the body has become sensitized to a particular antigen. Subsequent exposure of the skin to the antigen and the recognition of these antigens by T cells result in the release of various cytokines, proliferation and recruitment of T cells, and finally in dermatitis (eczema). If the causes can be identified, removal alone will cure allergic contact dermatitis. During active inflammation, topical corticosteroids are useful.

In anaphylaxis, or systemic immediate hypersensitivity, mast cell and basophil mediators gain access to vascular beds throughout the body and cause vasodilation and exudation of plasma. This in turn can lead to a fall in blood pressure, or shock, referred to as anaphylactic shock, which can be fatal. Anaphylactic shock usually results from the systemic presence of an antigen introduced by injection, an insect sting, or absorption across an epithelial surface, such as the skin or gut mucosa. Treatment is usually with systemic epinephrine, which can reverse the bronchoconstrictive and vasodilatory effects of the mediators.

The proteins of the STAT (signal transducers and activators of transcription) family are latent transcription factors that are abundantly expressed in many cell types. STAT6 is a ubiquitous transcription factor that is specifically activated following IL-4/IL-13 receptor mediated signaling, and acts at a point of convergence for genes regulated by these cytokines, including IgE. STAT6 deficient mice show markedly reduced IgE and Th2 cytokine production, and fail to develop antigen-induced airway hyper-reactivity in a model of airway inflammation (Kuperma et al. J. Exp. Med., 187:939-948, 1998). It has been demonstrated that STAT6 is obligatory for effective TH2 differentiation as well as for B cell class switching to IgE synthesis. In vivo liposome-mediated transfection of cis-element double stranded oligonucleotides (ODN) against STAT6 have been shown to inhibit both chronic and acute contact hypersensitivity in a mouse model (Sumi et al. Gene Ther. 11:1763-1771, 2004). These same STAT6 decoy ODNs have also been shown to have a significant inhibitory effect on the IgE-mediated late phase allergic response in a mouse model of atopic dermatitis (Yokozeki et al. Gene Ther. 11:1753-62-1771, 2004).

RNA interference (RNAi) is a post-transcriptional RNA silencing phenomenon used by most eukaryotic organisms as a defense mechanism against viral attack and transposable factors. This RNA silencing process was first identified in plants, where it is referred to as post-transcriptional gene silencing (PTGS), and was subsequently observed in the nematode C. elegans by Fire and Mello (Nature 391:806-811, 1998). RNAi involves the use of small interfering nucleic acid or RNA molecules (siRNAs) that selectively bind with complementary mRNA sequences, targeting them for degradation and thus inhibiting corresponding protein production. More recently it has been shown that siRNAs can induce de novo methylation and silencing of promoter sequences, known as transcriptional gene silencing (TGS).

More specifically, in an initiation step double-stranded RNA (dsRNA) is digested by the enzyme Dicer (a member of the RNase III family of dsRNA-specific ribonucleases) into small interfering RNAs (siRNAs) of 19-25 nucleotides in length. Each siRNA consists of two separate, annealed single strands of nucleotides, with each strand having a 2-3 nucleotide 3′ overhang. In the effector step, siRNA duplexes bind to a nuclease complex to form an RNA-induced silencing complex (RISC). The RISC then targets the endogenous mRNA complementary to the siRNA within the complex, and cleaves the endogenous mRNA approximately twelve nucleotides from the 3′ terminus of the siRNA. Degradation of the endogenous mRNA is then completed by exonucleases. An amplification step may also exist within the RNAi pathway in some organisms, for example by copying of the input dsRNAs or by replication of the siRNAs themselves.

Transfection of long dsRNA molecules of greater than 30 nucleotides into most mammalian cells causes nonspecific suppression of gene expression, as opposed to the gene-specific suppression seen in other organisms. This is believed to be due to activation of an antiviral defense mechanism that includes the production of interferon, and that leads to a global shut-down of protein production. However it has been shown that this pathway is not activated by dsRNAs less than 30 nucleotides in length, and that short dsRNAs of 21-23 nucleotides can be used to reduce specific gene expression in mammalian cells (Caplen et al., Proc. Natl. Acad. Sci. USA 17:9742-9747, 2001; Elbashir et al., Nature 6836:494-498, 2001). More recently, Brummelkamp et al. have demonstrated that siRNAs targeting oncogenes are effective in reducing tumors in mice (Cancer Cell 2:243-247, 2002).

RNAi has several advantages over other gene silencing techniques, such as the use of antisense oligonucleotides (ODN). RNAi techniques result in more specific inhibition of gene expression than ODN and are able to induce the same level of silencing as ODN at much lower concentrations of reagent. Also, siRNAs are more resistant to nuclease degradation than ODN. Bertrand et al. (Biochem. Biophys. Res. Commun. 296:1000, 2002) have shown that, in mice, siRNA silencing is more effective than antisense suppression.

It has been shown that sequence specificity of siRNA is important, as single base pair mismatches between the siRNA and its target mRNA can dramatically reduce silencing. Accordingly, in order to be effective in suppressing expression of a gene of interest to a high degree, siRNAs must be designed so that they are specific to the target gene. In addition, in order to avoid unwanted side effects, a delivery system must be employed that specifically delivers the siRNA to the desired target. Delivery of siRNA to cells by means of exogenous delivery of preformed siRNAs or via promoter-based expression of siRNAs or shRNAs has been described. Genetic constructs for the delivery of siRNA molecules are described, for example, in U.S. Pat. No. 6,573,099. The delivery of short RNA fragments to cells in vivo in mammals can be problematic due to the rapid degradation of the RNA. Short hairpin RNA (shRNA) are nucleic acid molecules that mimic the structure of the RNAi duplex and that can be produced in cells following delivery of expression vectors encoding the shRNA. The use of shRNA expression plasmids to reduce gene expression in vivo in rats has been described by Zhang et al., (J. Gene Med. 5:1039-1045, 2003).

### SUMMARY OF THE INVENTION

Briefly stated, the present invention provides compositions for the treatment and/or prevention of a disorder in a mammal by means of RNA interference, together with methods for the use of such compositions. Preferably, the disorder is an IgE-mediated disorder. In one aspect, the inventive compositions comprise: (a) a binding agent that specifically binds to a target internalizable antigen that is expressed on the surface of a target cell of interest, and (b) a small interfering nucleic acid molecule (siNA), that suppresses expression of a target gene within the target cell, whereby, after binding to the target antigen, the binding agent and siNA are internalized into the cell, and the siNA released.

In a related aspect, the present invention provides compositions comprising: (a) a binding agent that specifically binds to a target internalizable antigen that is expressed on the surface of a target cell of interest; and (b) a genetic construct that is capable of expressing a siNA that suppresses expression of a target gene within the target cell, whereby, after binding to the target antigen, the binding agent and genetic are internalized into the cell, and the siNA is expressed by the genetic construct. Preferably, the siNA is under the control of an RNA polymerase III or a tissue-specific RNA polymerase II promoter.

In a further aspect, the inventive compositions comprise a genetic construct that is capable of expressing a siNA that suppresses expression of a target gene within the target cell, wherein the genetic construct is packaged within a viral vector which, upon infection of the cell, releases its genetic material enabling expression of the genetic construct. Preferably the viral vector is an adenovirus-associated vector (AAV). In this aspect, viral capsid proteins may act as a binding agent.

In certain embodiments, the binding agent employed in the inventive compositions is an antibody, or an antigen-binding fragment thereof. Other binding agents that may be effectively employed in the inventive compositions include cell-specific ligands, and peptides or small molecules that specifically bind to cell-specific receptors. Viral (capsid) proteins may also be employed as binding agents.

In one embodiment, the binding agent is linked to the siNA, genetic construct or viral vector by means of a streptavidin-biotin linker as described below. In another embodiment, the siNA, genetic construct or viral vector is complexed to a lipid carrier, such as a cationic lipid carrier, which in turn is linked to the binding agent. In a related embodiment, the siNA, genetic construct or viral vector is encapsulated within a liposome, and the binding agent, or the antigen-binding portion thereof, is present on the surface of the liposome.

Preferably, the compositions of the present invention are effective in reducing expression of a gene that is active in a pathway involved in an IgE-mediated disease. In one such aspect, the siNA employed in the inventive compositions is capable of suppressing production of IgE in a cell that naturally expresses IgE, such as a B cell. In such compositions, the target antigen is an internalizable antigen that is expressed on the surface of a B cell, wherein binding of a complex to the antigen leads to internalization of the complex within the B cell. Preferably, the target antigen is CD19 or CD22. Examples of siNAs that are capable of suppressing expression of IgE include the siRNA sequences corresponding to the target sequences provided in SEQ ID NO: 12-100 and 824-915.

In a further aspect, the siNA employed in the inventive compositions is capable of suppressing expression of the high affinity receptor, FcεR1, and the binding agent specifically binds to a target antigen that is expressed on the surface of a mast cell or a basophil and that facilitates internalization of a complex bound to the target antigen. In one embodiment, the target antigen is FcεR1 itself, as FcεR1-bound complexes are known to be internalized and degraded by the cell. In another embodiment, the target antigen is the receptor CXCR4. Examples of siNAs that may be effectively employed in such compositions include the siRNA sequences corresponding to the target sequences provided in SEQ ID NO: 101-823.

In another aspect, the siNA employed in the inventive compositions is capable of suppressing expression of STAT6, and the binding agent specifically binds to a target antigen that is expressed on the surface of a cell that expresses STAT6 and that facilitates internalization of a complex bound to the target antigen. Delivery of compositions that are capable of suppressing expression of STAT6 is preferably targeted to haemopoietic cells, such as T cells, B cells, dendritic cells, macrophages and mast cells. Internalizable target antigens located on the surface of such cells include, for example, members of the integrin superfamily and cell adhesion molecules. The cDNA sequence for STAT6 is provided in SEQ ID NO: 942, with the sequence of the STAT6 promoter being provided in SEQ ID NO: 943. In certain embodiments, the inventive compositions that suppress expression of STAT6 comprise siNAs directed against non-coding untranslated regions (UTRs) of the STAT6 gene or the STAT6 promoter sequence. Examples of siNAs that are capable of suppressing expression of STAT6 include the siRNA sequences corresponding to the target sequences provided in SEQ ID NO: 944-980, wherein the target sequences of SEQ ID NO: 971-980 are directed to the STAT6 promoter.

In yet a further aspect, the siNA within the genetic construct is operably linked to a promoter that is specific to the target cell, whereby suppression of gene expression in non-target cells is reduced. For example, use of a promoter that drives the expression of a B cell specific antigen including, but not restricted to, immunoglobulin heavy chain (SEQ ID NO: 916, NCBI Locus ID HUMIGCC4), CD19 (SEQ ID NO: 917, NCBI Locus ID NM_{—}001770, corresponding genomic contig ID NT_{—}024812), CD20 (SEQ ID NO: 918, NCBI Locus ID for the cDNA sequence NM_{—}021950, for the protein sequence NP_{—}068769, corresponding genomic contig ID NC_{—}000011), CD21 (SEQ ID NO: 919, NCBI Locus ID AF298224), or CD22 (SEQ ID NO: 920, corresponding genomic sequence HSU62631) promoters, will prevent expression of the siNA in non-B cells.

In an alternative embodiment, the siNA employed in the inventive compositions is targeted against the promoter required for IgE chain synthesis (SEQ ID NO: 2) or against the promoters for IgE receptor genes (SEQ ID NO: 5 and 6, where SEQ ID NO: 6 is an exemplary fragment of the IgE beta receptor promoter), whereby introduction of the genetic construct into a target cell, such as a B cell, mast cell, or basophil, will lead to transcriptional gene silencing of the IgE or IgE receptor genes in the target cell.

In an additional embodiment, the siNA employed in the inventive compositions is targeted against the promoter or coding sequence of the recombinases required for IgE chain synthesis, whereby introduction of the genetic construct into a target cell, such as a B cell, will lead to reduction in the synthesis of IgE by the cell.

In yet other embodiments, the siNAs employed in the inventive compositions are targeted to intergenic/intronic regions flanking the IgE or IgE receptor genes/exons to be silenced, whereby introduction of the genetic construct into a target cell, such as a B cell, mast cell or basophil cell, will lead to partial or complete silencing of the desired genes. Preferably, by employing several siNAs in the inventive compositions which bind close to one another at unique sites in the target area, the degree of gene silencing can be controlled.

In a related aspect, the present invention provides methods for the prevention and treatment of an IgE-mediated disorder in a patient, comprising administering to the patient a composition of the present invention. IgE-mediated disorders that may be treated using the inventive methods include, but are not limited to, allergic rhinitis (e.g. hay fever), asthma, anaphylaxis, urticaria (hives), atopic dermatitis (eczema), food allergies, diseases that benefit from the reduction of eosinophilia in the tissues of the respiratory system, and disorders characterized by a hypersensitivity immune reaction.

In a related aspect, the present invention provides methods for the reduction of eosinophilia in a patient, such methods comprising administering at least one of the compositions disclosed herein. The reduction in eosinophilia will vary between about 20% and about 80%, preferably between 80% and 100%, and most preferably between 90% and 100%. The percentage of reduction in lung eosinophilia can be determined by measuring the number of eosinophils in bronchoalveolar lavage fluid before and after treatment.

In a further aspect, the present invention provides methods for modulating an IgE-mediated immune response to a specific antigen in a patient, comprising administering a composition of the present invention.

In yet another aspect, methods are provided for preventing or reducing the severity of an immune response to a specific antigen in a patient, comprising administering to the patient the specific antigen and a composition of the present invention. In a preferred embodiment, the specific antigen is an allergen. Preferably, the composition is administered at the time of sensitization or exposure of the patient to a specific antigen.

These and other aspects of the present invention will become apparent upon reference to the following detailed description. All references disclosed herein are hereby incorporated by reference in their entirety as if each was incorporated individually.

### BRIEF DESCRIPTION OF THE FIGURES

FIG. 1 shows an exemplary RNAi vector of the present invention.

FIG. 2 shows the inhibition of IgE expression by shRNAs in transfected HEK293T cells as determined by real time PCR.

FIG. 3 shows the inhibition of murine FcεRIβ expression by shRNAs in transfected HEK293T cells as determined by real time PCR.

FIG. 4 shows the inhibition of expression of a murine FcεRIβ/GFP fusion by shRNAs as determined by flow cytometry.

FIG. 5 shows intracellular IgE protein expression on transfected (eGFP⁺) IGEL b4 cells 48 hours after electroporation with an eGFP reporter plasmid and a pSilencer 2.1 plasmid expressing shRNA constructs designed to silence IgE expression.

FIG. 6 shows cell surface FcεRIα protein expression on transfected (eGFP⁺) versus non-transfected (eGFP⁻) MC/9 cells 48 hours after electroporation with an eGFP reporter plasmid and a pSilencer 2.1 plasmid expressing shRNA constructs designed to silence FcεRIβ expression.

### DETAILED DESCRIPTION OF THE INVENTION

As noted above, the present invention is generally directed to compositions and methods for the treatment of disorders that are mediated by IgE. In certain specific embodiments, such disorders are selected from the group consisting of: allergic rhinitis (e.g. hay fever), asthma, anaphylaxis, urticaria (hives), atopic dermatitis (eczema), food allergies, diseases that benefit from the reduction of eosinophilia in the tissues of the respiratory system, and disorders characterized by a hypersensitivity immune reaction.

The inventive compositions comprise a complex that includes: (a) a “naked” or modified small interfering nucleic acid molecule (siNA) directed against a target gene or a genetic construct that expresses the siNA under the control of a tissue-specific promoter; and (b) a binding agent, such as an antibody, that specifically binds to a target antigen which is present on the surface of a target cell of interest. The target antigen recognizes and internalizes certain specific biological molecules, such that, on binding of the siNA-antibody or genetic construct-antibody complex to the target antigen, the complex is internalized into the target cell by endocytosis, the siNA or genetic construct is released from the complex, and the siNA reduces expression of the target gene by means of RNA interference.

As used herein, the term “target gene” refers to a polynucleotide that comprises a region that encodes a polypeptide of interest, and/or a polynucleotide region that regulates replication, transcription, translation or other processes important to expression of the polypeptide.

As used herein, the term “small interfering nucleic acid molecule”, or siNA, refers to any nucleic acid molecule that is capable of modulating the expression of a gene by RNA interference (RNAi), and thus encompasses short interfering RNA (siRNA), short interfering DNA (siDNA), double-stranded RNA (dsRNA), double-stranded DNA (dsDNA), complementary RNA/DNA hybrids, nucleic acid molecules containing modified (semi-synthetic) base/nucleoside or nucleotide analogues (which may or may not be further modified by conjugation to non-nucleic acid molecules, custom modified primary or precursor microRNA (miRNA), short hairpin RNA (shRNA) molecules, and longer (up to one kb or more), dsRNA or hairpin RNA molecules, so long as these do not activate non-specific interference, for example via interferon. The hairpin region may be short (e.g. 6 nucleotides), long (undefined length), or may include an intron that is efficiently spliced in the targeted cells or tissues. Additionally, multiple tandem repeats in one orientation (for example, three or more short sense repeats) are included under the definition of siRNA, as these can elicit a potent RNAi like response in some systems.

Examples of siNAs that may be effectively employed in the inventive compositions and methods include those corresponding to the target DNA sequences provided in SEQ ID NO: 12-915 and 944-980. One of skill in the art will appreciate that, when comparing an RNA sequence to a DNA sequence, an RNA sequence will contain ribonucleotides where the DNA sequence contains deoxyribonucleotides, and further that the RNA sequence will typically contain a uracil at positions where the DNA sequence contains thymidine.

The term siRNA will be used in this disclosure as a prototypical small interfering nucleic acid molecule.

In some embodiments the siNA is generated from an introduced DNA molecule that contains promoter and terminator sequences responsible for transcribing the nucleic acid sequences that comprise the siNA. The introduced DNA may be in the form of a covalently-closed linear or circular plasmid or a PCR product, and these will preferably contain little or no DNA of prokaryotic origin. DNA constructs may also contain a nuclear localization sequence, such as that derived from the SV40 enhancer, to promote nuclear uptake and expression of the construct. Promoters may be of the type activated by RNA polymerase III or RNA polymerase II. Those of the former type include U6, tRNAval, H1, and versions of these promoters modified to achieve higher levels of transcription. Promoters activated by RNA polymerase II may be constitutive (such as the widely used CMV and EF1α promoters), or may be transcribed in a preferred manner in a single cell, cell type, tissue type, or biochemical event. These latter promoters may be chosen for high level or low-level expression. When a hairpin or custom miRNA is used, a single specific promoter may be employed. When two custom microRNAs with complementary target regions are employed, or when dsRNAs are to be formed from two separate strands, combinations of constitutive and specific, or specific promoters may be employed. In circumstances where reduced, but not eliminated, expression levels are desired, this may be achieved using completely or incompletely homologous antisense siNAs, or using promoters of varying transcriptional activity. Alternatively, siNA may be targeted to regions of mRNA that are either highly affected, or less completely affected, by an siNA, or more than one siNA sequence directed to the target gene, genes or a pathway may be used to achieve stronger interference.

The siNA may be targeted to the 5′ untranslated region, the coding region, or the 3′ untranslated region of the target gene or message. Additionally, regions of the promoter of a target gene, or regions usually upstream of a gene may be targeted for RNAi assisted heterochromatin formation.

An siNA can be unmodified or may be chemically-modified in order to increase resistance to nuclease degradation as described, for example, in International Patent Publication nos. WO 03/070970 and WO 03/074654. Thus, for example, some or all of the nucleotides of an siNA may comprise modified nucleic acid residues, or analogs of nucleic acid residues. The hybridization characteristics of the modified siNA may be similar to or improved compared to the corresponding unmodified siNA. Such modifications can also improve the efficacy and safety of in vivo therapy by changing the stability, lifetime and circulation of the siNAs in the human body. Preferably the siNA is between 19 to 30 nucleotides in length (for example, 19, 20, 21, 22, 23, 24, 25, 26, 27, 28, 29 or 30 nucleotides), more preferably 19-25 nucleotides in length, and most preferably 21 to 23 nucleotides in length, and comprises an antisense strand that is complementary to at least a portion of a nucleotide sequence, such as a mRNA sequence corresponding to a target DNA sequence. The siNA may also contain a sense strand that comprises the portion of the nucleotide sequence of interest. The sense and antisense strands may be separate, distinct sequences, as in a dsRNA molecule, or may be linked as, for example, in a shRNA molecule.

Those skilled in the art will appreciate that minor changes in the sequence of the siNAs directed against target sequences disclosed herein can yield siNAs that hybridize strongly and specifically to the target nucleic acid. For example, siNAs directed against target sequences that are shifted by one to four nucleotides 5′ or 3′ of the sequences disclosed herein may be effective. It is useful to administer more than one such variant to a target area, or a combination of several different siNAs targeting different regions in and around the desired gene (e.g., exons, introns, promoter, or intergenic regions).

Preferably the siNA is targeted against a gene or nucleotide sequence that functions in a pathway that is involved in IgE-mediated disorders. In specific embodiments, the siNA employed in the inventive compositions and methods is targeted against one or more subsequences in: (1) an mRNA molecule that encodes IgE or a portion thereof, such as the epsilon heavy chain constant region (SEQ ID NO: 1, Genbank No. X83965); (2) an IgE promoter sequence (SEQ ID NO: 2, corresponding to NCBI human genome assembly chromosome 14 coordinates 104037954-104426844); (3) an mRNA molecule that encodes the high affinity FcεR1 receptor alpha subunit (SEQ ID NO: 3; GenBank/EMBL entry X06948); (4) an mRNA molecule that encodes the high affinity FcεR1 receptor beta subunit (SEQ ID NO: 4; Genbank/EMBL ID D10583); (5) an FcεR1 receptor alpha subunit promoter sequence (SEQ ID NO: 5, corresponding to NCBI human chromosome 1 coordinates 156474296-156488950); (6) an FcεR1 receptor beta promoter (such as SEQ ID NO: 6, in the region upstream of start codon in SEQ ID NO: 4); (7) the 3-prime UTR of the FcεR1 beta receptor (SEQ ID NO: 7, Genbank/EMBL ID 3HSA025677-3UTR); (8) IgE epsilon domains 1-4 (SEQ ID NO: 8-11, respectively); and (9) non-coding untranslated regions (UTRs) of the STAT6 gene or the STAT6 promoter sequence. In other embodiments the siNA is directed against molecules involved in the processing of IgE such as the recombinases responsible for immunoglobulin class switch recombination.

Methods for selecting suitable regions in a mRNA target are disclosed in the art (see, for example, Vickers et al., J. Biol. Chem. 278:7108-7118, 2003; Elbashir et al., Nature 411:494-498, 2001; Elbashir et al., Genes Dev. 15:188-200, 2001). Preferably, selected target sequences are sensitive to down regulation by low concentrations of siRNA. Guidelines for the design of siNA include those provided in Ambion's Technical Bulletin #506 (available from Ambion Inc., Austin, Tex.), and are described below. The use of low concentrations of siRNA (for example, nanomolar or sub-nanomolar concentrations) and avoidance of sequences that occur in alternative spliced gene products is important for limiting off-target, non-sequence specific, effects. Assessing whether a gene has been downregulated, and the extent of downregulation, can be performed using, for example, real-time PCR, PCR, western blotting, flow cytometry or ELISA methods.

Methods for the preparation of genetic constructs, or expression vectors, comprising, or encoding, siNA targeted against nucleotide sequences of interest are detailed below.

As used herein, the term “binding agent”, refers to a molecule that specifically binds to a target antigen expressed on the surface of a target cells, and includes, but is not limited to, antibodies, including monoclonal antibodies and polyclonal antibodies; antigen-binding fragments thereof, such as F(ab) fragments, F(ab′)₂ fragments, variable domain fragments (Fv), small chain antibody variable domain fragments (scFv), and heavy chain variable domains (V_{HH}); small molecules; hormones; cytokines; ligands; peptides and viruses (either native or modified). Antibodies, and fragments thereof, may be derived from any species, including humans, or may be formed as chimeric proteins which employ sequences from more than one species. The term “binding agent” as used herein thus encompasses humanized antibodies and veneered antibodies.

A binding agent is said to “specifically bind,” to a target antigen if it reacts at a detectable level (within, for example, an ELISA assay) with the target antigen, and does not react detectably with unrelated antigens under similar conditions.

Antibodies, and fragments thereof, may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described, for example, by Kohler and Milstein, Eur. J. Immunol. 6:511-519, 1976, and improvements thereto, via transfection of antibody genes into suitable bacterial or mammalian cell hosts, in order to allow for the production of recombinant antibodies, or by protein synthesis.

In order to minimize any off-target effects, the binding agents employed in the inventive compositions and methods are preferably cell type-specific. For example, for complexes containing siNA targeted against genes involved in IgE expression, the binding agent is specific for internalizable cell surface antigens found on B cells, such as CD19 and CD22. For complexes containing siNA targeted against genes involved in expression of the FcεR1 receptor, the binding agent is specific for internalizable cell surface antigens found on mast cells and/or basophils, such as FcεR1 itself or CXCR4. Examples of binding agents that may be usefully employed in the present invention include: anti-human CD19 antibodies, anti-murine CD19 antibodies, anti-human CD22 antibodies, anti-murine CD22, anti-human FcεR1 antibodies, anti-murine FcεR1 antibodies, anti-human CXCR4 antibodies, anti-murine CXCR4 antibodies, anti-transferrin antibodies, and antigen-binding fragments thereof.

Other binding agents that may be effectively used in the inventive compositions and inventions include the CXCR4-specific chemokine ligand CXCL12 (also known as SDF-1α), CXCR4-binding peptides derived from CXCL12, other peptides that specifically bind to CXCR4, and small molecules or drugs that bind to CXCR4. One example of a drug that binds CXCR4 is the distamycin analog 2,2′[4,4′-[[aminocarbonyl]amino]bis[N,4′-di[pyrrole-2-carboxamide-1,1′-dimethyl]]-6,8 napthalene disulfonic acid] hexasodium salt, also referred to as NSC651016. NSC651016 has been shown to specifically inhibit binding of chemokines to the receptors CXCR4, CCR5, CCR3 and CCR1. In addition, binding of NSC651016 to CXCR4 and to CCR5 has been demonstrated to induce receptor internalization and delayed recycling of the receptors (Howard et al., J. Leukoc. Biol. 64:6-13, 1998). NSC651016 is also known to have anti-inflammatory and anti-angiogenesis activities.

One example of a peptide that binds to CXCR4, and therefore may be usefully employed as a binding agent in the inventive compositions to target mast cells, is T22 ([Tyr^{5,12},Lys⁷]-polyphemusin II; Murakami et al., J. Exp. Med. 186:1389-1393, 1997). T22 is a synthetic 18 amino acid peptide analog of polyphemusin II isolated from the hemocyte debris of American horseshoe crabs which has been shown to prevent infection by HIV-1 isolates by blockage of CXCR4. Another example of a binding agent that may be used to target mast cells by binding to CXCR4 is N-α-acetyl-nona-d-arginine (Arg) amide (ALX40-4C; Doranz et al., J. Exp. Med. 186:1395-1400, 1997) which has been shown to have a high degree of selectively for CXCR4 and to block infection by HIV-1 strains at low, micromolar, concentrations. Other small molecules that may be usefully employed as binding agents to target mast cells include the CXCR4 antagonists AMD3100 (a heterocylic bicyclam derivative) and AMD070, both available from AnorMED Inc., Vancouver, Canada. Blockage of CXCR4 with the soluble inhibitor AMD3100 has been shown to reduce a number of pathological parameters related to asthmatic-type inflammation in a mouse model (Lukacs et al., Am. J. Pathol. 160:1353-1360, 2002) and to inhibit autoimmune joint inflammation in a IFN-γ receptor-deficient mice (Matthys et al. J. Immunol. 167:4686-4692, 2001).

In one embodiment, the compositions of the present invention comprise a binding agent, such as an antibody, connected to a genetic construct by means of a streptavidin-biotin linkage. As used herein, the term “streptavidin” encompasses both streptavidin and avidin, and derivatives or analogues thereof that are capable of high affinity, multivalent or univalent binding of biotin. Techniques for the preparation of conjugates containing streptavidin-biotin linkages are well known in the art and include, for example, those described in U.S. Pat. Nos. 6,287,792 and 6,217,869, the disclosures of which are hereby incorporated by reference. Biotin may be incorporated into the genetic construct using, for example Biotin-21-dUTP™ (BD Biosciences Clontech, Palo Alto, Calif.), which is a dTTP analog with biotin covalently attached to the pyrimidine ring through a 21-atom spacer arm. The biotin-labeled genetic construct is then linked to the streptavidin-antibody conjugate via biotin-streptavidin binding, using techniques well known to those of skill in the art. Streptavidin-biotin linkers may, alternatively, be employed to link binding agent directly to “naked” siNA.

In a further embodiment, the present invention provides complexes that comprise a binding agent, such as an antibody, and a polynucleotide-binding component, such as a polycation, that is covalently bonded to the antibody through, for example, disulfide bonds. Polycations that may be employed as polynucleotide-binding components include, for example, polylysine, polyarginine, polyornithine, and basic proteins, such as histones, avidin and protamines. The polynucleotide-binding component is then attached to a genetic construct by means of electrostatic attraction between the opposite charges present on the genetic construct and the polynucleotide-binding component. The antibody is thus bound to the genetic construct without functionally altering either the genetic construct or the antibody. Both the bond between the antibody and the polynucleotide-binding components and that between the polynucleotide-binding component and the genetic construct are cleaved following internalization of the complex into the target cell. Such complexes may be prepared as described, for example, in U.S. Pat. No. 5,166,320.

Cleavable polymeric linkers which may be effectively employed to attach a genetic construct of the present invention to a binding agent are also described in U.S. Pat. No. 6,627,616.

Alternatively, helicases and other RNA-binding proteins, may be linked to the binding agent, or antibody, and naked siNA is, in turn, linked to the helicase prior to administration. Examples of such helicases and RNA-binding proteins are provided in Sasaki et al., Genomics 82:323-330, 2003, Yan et al., Nature 426:469-474, 2003 and Anderson et al., Mol. Cell Proteomics, Manuscript M300127-MCP200, Epub Jan. 12 2004.

In an alternative embodiment, the genetic construct of the present invention is encapsulated in a liposome or polymer, or attached to a lipid or polymer carrier, which is in turn attached to a binding agent, such as an antibody directed against the target antigen. Encapsulation of the genetic construct within a liposome protects the construct from degradation by endonucleases. Methods for the encapsulation of biologically active molecules, such as nucleic acid molecules and proteins, within liposomes or polymers, and for the preparation of nucleic acid-lipid (lipoplex) and nucleic acid-polymer (polyplex) carrier complexes are well known in the art. See, for example, U.S. Pat. Nos. 6,627,615, 4,241,046, 4,235,871 and 4,394,448; and Liposome Technology: Liposome Preparation and Related Techniques, ed. G. Gregoriadis, CRC Press, 1992. Liposome formulation, development and manufacturing services are available for example, from Gilead Liposome Technology Group (Foster City, Calif.). Lipids for the preparation of liposomes are available, for example from Avanti Polar Lipids, Inc. (Alabaster, Ala.).

The resulting liposome carrier containing the genetic construct of interest is then conjugated to the binding agent, using methods well known in the art, such as those taught, for example, in U.S. Pat. Nos. 5,210,040, 4,925,661, 4,806,466 and 4,762,915. Such methods include the use of linkers that fall into three major classes of functionality: conjugation through amide bond formation; disulfide or thioether formation or biotin-streptavidin binding. In a preferred embodiment, the liposome is attached to the binding agent, such as an antibody, by means of a maleimide linker, as described, for example, in U.S. Pat. No. 6,372,250, the disclosure of which is hereby incorporated by reference.

In a preferred embodiment, the liposome employed in the inventive compositions is a pegylated liposome, wherein the surface of the liposome is conjugated with multiple (up to several thousand) strands of poly(ethylene glycol) (PEG) of approx. 2000 Da. The binding agent is then conjugated to the tips of some of the PEG strands. The diameter of the liposome is preferably within the range of 100 nm to 10 μm. The preparation of such pegylated liposomes and attachment of monoclonal antibodies to the liposomes is performed as described, for example, in Shi and Pardridge, Proc. Natl. Acad. Sci. USA 97:7567-7572, 2000; and Shi et al., Proc. Natl. Acad. Sci. USA 98:12754-12759, 2000. Pegylation of the liposome should increase the stability of the liposome and prevent non-specific attachment of cells, such as macrophages, and proteins to the liposome. The preparation of pegylated liposomes, which encapsulate shRNA expression plasmids and are conjugated to monoclonal antibodies, and the use of such compositions in vivo in silencing gene expression in brain cancer is described in Zhang et al., J. Gene Med. 5:1039-1045, 2003.

Alternatively, the siNA or genetic construct of the present invention is packaged in an adenovirus or adeno-associated virus vector, which upon infection of the cell releases its genetic material enabling construct expression. In this embodiment, viral capsid proteins may act as the binding agent and target the siNA or genetic construct to specific cells.

Adenoviruses (AV) and adeno-associated viruses (AAV) do not integrate their genetic material into the host genome and do not require host replication for gene expression. AV and AAV vector delivery systems are thus well suited for rapid and efficient, transient expression of heterologous genes in a host cell. AAV vector delivery systems have previously been shown to be effective in the treatment of cystic fibrosis (Aitken et al., Hum. Gene Ther. 12:1907-1916, 2001). Examples of AAV vector delivery systems which may be effectively employed in the present invention include, but are not limited to, those described in U.S. Pat. No. 6,642,051 and references cited therein. Improvements have been made in the efficiency of targeting adenoviral vectors to specific cells by, for example, coupling adenovirus to DNA-polylysine complexes and by strategies that exploit receptor-mediated endocytosis for selective targeting. See, e.g., Curiel et al., Hum. Gene Ther. 3:147-154 (1992); and Cristiano and Curiel, Cancer Gene Ther. 3:49-57 (1996). Alternatively, for situations where stable transfection is desired, viral vectors that insert genetic material into a host cell's genome may be employed. Examples of such vectors include lentiviral, retroviral, plasmid and MLV vectors. The design and use of lentiviral vectors suitable for gene therapy is described, for example, in U.S. Pat. Nos. 6,531,123, 6,207,455 and 6,165,782, the disclosures of which are hereby incorporated by reference. The use of lentivector-delivered RNA interference in silencing gene expression in transgenic mice is described by Rubinson et al. (Nat. Genet. 33:401-406, 2003).

The present invention further provides methods for the treatment of IgE-mediated disorders in a patient by administration of a therapeutically effective amount of a composition disclosed herein.

As used herein, a “patient” refers to any warm-blooded animal, including, but not limited to, a human. Such a patient may be afflicted with disease or may be free of detectable disease. In other words, the inventive methods may be employed for the prevention or treatment of disease. The inventive methods may also be employed in conjunction with other known therapies.

In general, the inventive compositions may be administered by injection (e.g., intradermal, intramuscular, intravenous or subcutaneous), intranasally (e.g., by aspiration), orally or epicutaneously (applied topically onto skin). In one embodiment, the compositions of the present invention are in a form suitable for delivery to the mucosal surfaces of the airways leading to or within the lungs. For example, the composition may be suspended in a liquid formulation for delivery to a patient in an aerosol form or by means of a nebulizer device similar to those currently employed in the treatment of asthma.

For use in therapeutic methods, the inventive compositions may additionally contain a physiologically acceptable carrier. While any suitable carrier known to those of ordinary skill in the art may be employed in the compositions of this invention, the type of carrier will vary depending on the mode of administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose and magnesium carbonate, may be employed. Biodegradable microspheres (e.g., polylactic galactide) may also be employed as carriers for the compositions of this invention. Suitable biodegradable microspheres are disclosed, for example, in U.S. Pat. Nos. 4,897,268 and 5,075,109. Other components, such as buffers, stabilizers, biocides, etc., may be included in the inventive compositions.

The preferred frequency of administration and effective dosage will vary from one individual to another and will depend upon the particular disease being treated and may be determined by one skilled in the art. Preferably, the dosage is sufficient to provide siNA at a concentration of between 1 nM and 100 nM. The inventive compositions may be administered in a single dosage, or in multiple, divided dosages. The inventive compositions may be employed in combination with one or more known therapeutic agents.

The following Examples are offered by way of illustration and not by way of limitation.

### EXAMPLE 1

### Preparation of Antibody-Conjugated Liposomes

Preparation of pegylated liposomes, encapsulation of genetic constructs and conjugation with monoclonal antibody may be carried out as follows.

1-Palmitoyl-2-oleoyl-sn-glycerol-3-phosphocholine (POPC; Avanti Polar Lipids, Alabaster Ala.; 19.2 μmol), didodecyldimethylammonium bromide (DDAB; Avanti Polar Lipids; 0.2 μmol), distearolyphosphatidylethanolamine ((DSPE)-PEG 2000; Shearwater Polymers, Huntsville, Ala.; 0.6 μmol) and DSPE-PEG 2000-maleimide (30 nmol) are dissolved in chloroform/methanol (2:1, vol:vol) followed by evaporation. The lipids are dispersed in 1 ml 0.05 M Tris-HCl buffer (pH=8.0) and sonicated for 10 min. Supercoiled plasmid DNA is added to the lipids and the liposome/DNA dispersion evaporated to a final concentration of 200 mM at a volume of 100 μl. The dispersion is frozen in ethanol/dry ice for 4-5 min and thawed at 40° C. for 1-2 min. This freeze-thaw cycle is repeated 10 times. The liposome dispersion is then diluted to a lipid concentration of 40 mM, followed by extrusion 10 times each through two stacks of polycarbonate filter membranes. The mean vesicle diameters may be determined using a Microtrac Ultrafine Particle Analyzer (Leeds-Northrup, St. Petersburg, Fla.).

Plasmid attached to the exterior of the liposomes is removed by nuclease digestion as described by Monnard et al. (Biochim. Biophys. Acta 1329:39-50, 1997). For digestion of the unencapsulated DNA, 5 units of pancreatic endonuclease I and 5 units of exonuclease II are added in 5 mM MgCl₂ and 0.1 mM DTT to the liposome/DNA mixture after extrusion. After incubation at 37° C. for 1 h, the reaction is stopped by adding 7 mM EDTA.

Monoclonal antibody specific for the target antigen is thiolated using a 40:1 molar excess of 2-iminothiolane (Traut's reagent) as described by Huwyler et al., Proc. Natl. Acad. Sci. USA 93:14164-14169, 1996. Thiolated antibody is then incubated with the liposomes overnight at room temperature, and the resulting immunoliposomes are separated from free monoclonal antibody by, for example, gel filtration chromatography.

### EXAMPLE 2

### Design of SiRNA Oligonucleotides Directed Against the Fc Fragment of IgE

The DNA sequences encoding for the CH1, CH2, CH3 and CH4 domains of human IgE are provided in SEQ ID NO: 8, 9, 10 and 11, respectively.

Potential target sites in the mRNA are identified based on rational design principles, which include target accessibility and secondary structure prediction. Each of these may affect the reproducibility and degree of knockdown of expression of the mRNA target, and the concentration of siRNA required for therapeutic effect. In addition, the thermodynamic stability of the siRNA duplex (e.g., antisense siRNA binding energy, internal stability profiles, and differential stability of siRNA duplex ends) may be correlated with its ability to produce RNA interference. (Schwarz et al., Cell 115:199-208, 2003; Khvorova et al., Cell 115:209-216, 2003). Empirical rules, such as those provided by the Tuschl laboratory (Elbashir et al., Nature 411:494-498, 2001; Elbashir et al., Genes Dev. 15:188-200, 2001) are also used. Software and internet interactive services for siRNA design are available at the Ambion and Invitrogen websites. Levenkova et al. describe a software system for design and prioritization of siRNA oligos (Levenkova et al., Bioinformatics 20:430-432, 2004). The Levenkova system is available on the internet and is downloadable freely for both academic and commercial purposes. The siRNA molecules disclosed herein were based on the Ambion, Invitrogen and Levenkova recommendations.

The selection of siRNA oligos disclosed in this application was based primarily on uniqueness vs human sequences (i.e., a single good hit vs human Unigene, and a big difference in hybridization temperature Tm against the second best hit) and on GC content (i.e., sequences with % GC in the range of 40-60%).

Optionally, for a more detailed picture on the potential hybridization of the oligos, RNA target accessibility and secondary structure prediction can be carried out using, for example, Sfold software (Ding Y and Lawrence, C. E. (2004) Rational design of siRNAs with Sfold software. In: RNA Interference: from Basic Science to Drug Development. K. Appasani (Ed.), Cambridge University Press; Ding and Lawrence, Nucleic Acids Res. 29:1034-1046, 2001; Nucleic Acids Res. 31:7280-7301, 2003). Sfold is available on the internet. RNA secondary structure determination is also described in Current Protocols in Nucleic Acid Chemistry, Beaucage et al., ed, 2000, at 11.2.1-11.2.10.

The targeted region is selected from a cDNA sequence, such as the CH1, CH2, CH3 or CH4 sequence of IgE. Potential target sequences and positions are typically identified by searching for specific 23 nucleotide (nt) motifs (“Tuschl patterns” such as AA(N19)TT, where N is any nucleotide, and AA is referred to herein as the “target motif leader”, NA(N21), or BA (N21), where B=C,G,U; Elbashir S M et al., Methods 26:199-213, 2002) in the cDNA sequence, starting at about 50-100 nt downstream of the start codon. The nt 22 and nt 23 need not be considered in searching for Tuschl patterns, since they are not involved in the base pairing between the mRNA target and the antisense siRNA strand. “Sense siRNA” is used herein to mean a target sequence without the NN leader. For example, the sequence of the sense siRNA corresponds to (N19)TT of the Tuschl pattern AA(N19)TT (positions 3-23 if the 23 nt motif).

The siRNAs are preferably designed with symmetric 3′ overhangs in order to form a symmetric duplex. For both sense and antisense siRNAs, either dTdT or UU are used as the 3′ overhang. Thus for siRNAs with an AA target motif leader, the AA base pairs with the dTdT or UU overhang of the antisense siRNA. For BA leaders, the A pairs with the first dT or U of the overhang. It is known however, that the overhang of the sense sequence can be modified without affecting targeted mRNA recognition.

The antisense siRNA is synthesized as the complement to position 1-21 of the 23 nt motif. The 3′ most nucleotide can be varied, but the nucleotide at position 2 of the 23 nt motif is selected to be complementary to the targeted sequence. These methods are well known in the art. Where it is desired to efficiently express RNAs from pol III promoters, the first transcribed nt should be a purine. For example, the siRNA may be selected corresponding to the target motif NAR (N17) YNN, where R is (A,G) and Y is (C,U). Preferably the siRNAs are designed with symmetric 3′TT overhangs (Elbashir et al., EMBO J. 20:6877-6888, 2001).

The target sequence motifs are selected to have about 30-70% GC content, preferably 40-60% GC content. As used herein, the “% GC” is calculated as: [the number of G or C nucleotides in the target sequence/21 for an AA target motif leader]×100, [the number of G or C nucleotides in the target sequence/20 for a BA target motif leader]×100, and [the number of G or C nucleotides in the target sequence/19 for an NB target motif leader]×100.

Following selection of siRNA duplexes from the target sequence, the thermodynamic properties of the sequences are determined, e.g., using the Sfold software referred to above. As used herein, “DSSE” refers to the differential stability of the siRNA duplex ends, i.e., the average difference between 5′ antisense and 5′ sense free energy values for the four nucleotide base pairs at the ends of the duplex. It has been shown that the 5′AS region is less stable than the 5′S terminus in functional siRNA duplexes and vice versa for nonfunctional siRNA duplexes (Khvorova et al., Cell 115:209-216, 2003). It is known that the siRNA duplex can be functionally asymmetric, in the sense that one of the two strands preferentially triggers RNAi (Schwartz et al., Cell 155:199-208, 2003).

As used herein, “AIS” refers to the average internal stability of the duplex at positions 9-14 from the 5′ end of the antisense strand. Comparisons between functional and nonfunctional siRNA duplexes indicate that the functional siRNA has lower internal stability in this reason. It is proposed that flexibility in this region may be important for target cleavage (the mRNA is cleaved between position 9 and 10) and/or release of cleaved products from RISC to regenerate RISC. See Khvorova et al., Cell 115:209-216, 2003).

The siRNA sequences directed against the CH1, CH2, CH3 and CH4 domains of IgE and their thermodynamic properties are further selected according to the following criteria: (a) 40%≦GC content≦60%; (b) antisense siRNA binding energy≦−15 kcal/mol; and (c) exclusion of target sequence with at least one of AAAA, CCCC, GGGG or UUUU. For siRNAs with NN dinucleotide leaders, two additional criteria are used: (d) DSSE>0 kcal/mol (Zamore asymmetry rule); and (e) AIS>−8.6 kcal/mol (cleavage site instability rule). This is the midpoint between the minimum of −3.6 and maximum of −13.6 (Khvorova et al., 2003).

Exemplary siRNAs for domains CH1, CH2, CH3 and CH4 are siRNA sequences corresponding to the target sequences provided in SEQ ID NO: 886-915.

In like manner, siRNA duplexes are designed against the human Fc Igε high affinity receptor α chain target sequences (SEQ ID NO: 649-686) and the human Fc Igε high affinity receptor β chain target sequences (SEQ ID NO: 687-708).

To increase the likelihood that only one gene will be targeted for degradation, the selected siRNA sequences are further checked for uniqueness against human and murine gene libraries (e.g., TIGR GI, ENSEMBL human genome), using Blast algorithms. Also, to increase the likelihood that the selected sequences will be active, sequences directed against targets having SNPs in the base pairing regions are excluded.

### EXAMPLE 3

### Synthesis and Testing of SiRNA Duplexes

SiRNA may be prepared by various methods, e.g., chemical synthesis, or from suitable templates using in vitro transcription, siRNA expression vectors or PCR generated siRNA expression cassettes. Preferably, chemical synthesis is used.

Methods for chemical synthesis of RNA are well known in the art and are described, for example, in Usman et al., J. Am. Chem. Soc. 109:7845, 1987; Scaringe et al., Nucleic Acids Res. 18:5433, 1990; Wincott et al., Nucleic Acids Res. 23:2677-2684, 1995; and Wincott et al., Methods Mol. Biol. 74:59, 1997. 21-nt siRNAs with 3′ overhangs may be synthesized, for example, using protected ribonucleoside phosphoramidites and a DNA/RNA synthesizer, and are commercially available from a number of suppliers, such as Proligo (Hamburg, Germany), Dharmacon Research (Lafayette, Colo.), Perbio Science (Rockford, Ill.), Glen Research (Sterling, Va.), ChemGenes (Ashland, Mass.), and Ambion Inc. (Austin, Tex.). The siRNA strands can then be deprotected, annealed and purified before use, if necessary. Annealing can be carried out, for example, by incubating single-stranded 21-nt RNAs in 100 mM potassium acetate, 30 mM HEPES-KOH at pH 7.4, 2 mM Mg acetate, 1 min at 90° C., then 1 hr at 37° C. The solution is then stored frozen at −20° C. Useful protocols can be found in Elbashir et al., Methods 26:199-213, 2002.

### EXAMPLE 4

### RNAi Expression Vectors

Expression vectors for generating siRNA fragments targeting IgE or the IgE receptor FcεR1 are constructed by ligating annealed, chemically synthesized, oligonucleotide pairs into the appropriate vector (pSilencer, pSiren), or by PCR amplification of cDNA corresponding to siRNA sequences. For expression from vectors, siRNA sequences should start with 5′G residues. Symmetric 3′ overhangs and appropriate restriction sequences are added during amplification. The amplified sequences are subcloned into, for example, pcDNA3 vectors (Invitrogen, San Diego, Calif.).

B cell promoters, prepared from database sequences for IgG1 (SEQ ID NO: 916, NCBI Locus ID HUMIGCC4), CD19 (SEQ ID NO: 917, NCBI Locus ID NM_{—}001770 with corresponding genomic contig ID NT_{—}024812), CD20 (SEQ ID NO: 918, NCBI Locus ID NM_{—}021950 with corresponding genomic contig ID NC_{—}000011), CD21 (SEQ ID NO: 919, NCBI Locus ID AF298224 containing promoter and 5′UTR), and CD22 (SEQ ID NO: 920, NCBI Locus ID NM_{—}001771 and genomic sequence HSU62631) promoters, and mast cell promoters, such as the chymase promoter (SEQ ID NO: 924, NCBI Locus ID NM_{—}001836, corresponding genomic contig NT_{—}026437), tryptase promoters (tryptase alpha, SEQ ID NO: 921, NCBI Locus ID NM_{—}003293 having corresponding genomic segment containing promoter area NT_{—}037887; tryptase beta 1, SEQ ID NO: 922, NCBI Locus ID NM_{—}003294 having corresponding genomic segment containing promoter area NT_{—}037887; tryptase beta 2, SEQ ID NO: 923, NCBI Locus ID NM_{—}024164 having corresponding genomic segment containing promoter area NT_{—}037887), or FcεR1 promoters (SEQ ID NO: 5 and 6), are cloned into expression vectors containing a fluorescent reporter gene, such as EGFP, and tested in human and murine B and T cell lines, or mast cell lines (American Type Culture Collection (ATCC), Manassas, Va., No. CRL-8306), for their ability to confer B cell-specific expression or mast cell-specific expression, respectively. Based on these experiments, appropriate promoters are selected and subcloned into IgE-specific and FcεR1-specific RNAi vectors.

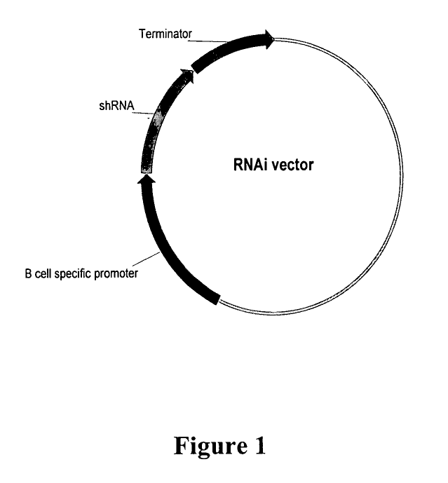

An exemplary RNAi vector is shown in FIG. 1. The vector can be constructed based on commercially available vectors such as pSilencer from Ambion and comparable vectors from other suppliers. Alternatively, covalently-closed linear constructs, containing only the shRNA expression cassette, can be used. These constructs can be generated by PCR or restriction digestion followed by ligation of short hairpin oligos to yield endonuclease-resistant covalently-closed molecules. In a further embodiment, these constructs may contain nuclear localization sequences to promote nuclear uptake and expression of the construct.

### EXAMPLE 5

### RNAi-Directed Transcriptional Silencing

For long-term suppression of IgE expression, it would be advantageous to silence the transcription of IgE by producing double-stranded RNAi in the nucleus that is capable of triggering transcriptional gene silencing of IgE gene expression. This may be done by introducing RNAi constructs into B cells that are expressed in the nucleus, and that contain promoter sequences directed against the IgE promoter or the promoter of a transcription factor that activates the IgE promoter. RNAi-dependent chromatin silencing has been demonstrated in both fission yeast and plants (reviewed by Matzke and Matzke, Science 301:1060-1061, 2003). In plants, the synthesis of double-stranded RNA containing promoter sequences triggers transcriptional gene silencing and methylation of the target promoter (Mette et al., EMBO J. 19:5194-5201, 2000).

Expression cassettes are designed to express siRNAs in the nucleus under the control of a human U6 snRNA promoter or tissue specific promoters such as the IgH, CD19, CD20, CD21 or CD22 promoter. See, e.g., Miyagishi and Taira, Nature Biotechnology 20:497-500, 2002; Paul et al., ibid, 505-508). The cassette also contains U6 termination sequences. The desired IgE promoter sequences or IgE transcription factor sequences are subcloned into the cassette, e.g., a pU6 plasmid, or a linear derivative of such a plasmid. To promote nuclear uptake, these constructs can be engineered to include nuclear localization sequences. Various strategies may be tested, including the production of short hairpin siRNAs containing one or more inverted DNA repeats and/or tandem DNA repeats of promoter-containing sequences, and synthesis of separate sense and antisense promoter RNAs in a single construct with two different promoters.

Guidelines for constructing hairpin siRNA expression cassettes may be found, for example, in the Ambion Technical Bulletin #506 (Ambion Inc, Austin, Tex.).

Chromatin silencing in cells transfected with nuclear-targeted siRNA vectors is assessed by methods to detect gene-specific mRNA or protein expression such as quantitative PCR, Northern blotting, ELISA, flow cytometry and western blotting.

### EXAMPLE 6

### SiRNA Mediated Silencing of IgE and FcεRI Expression in Vitro

The ability of siRNAs/shRNAs to downregulate their target sequences may be tested in a model system by co-transfection of a cDNA encoding the target message and the siRNA/shRNA to be tested as detailed below. Such systems comprise an easily transfectable cell line, e.g. HEK293. The activity of selected siRNA sequences against endogenously expressed target genes may be tested by transfecting primary B cells, mast cells, or cell lines derived from these cell types, in vitro using commercially available transfection reagents (for example, Lipofectamine 2000, Invitrogen), electroporation (BTX ECM600), lipid-based complexes without targeting, or more specifically with transferrin receptor- and CD19-specific antibody-liposome complexes containing siRNA.

a) SiRNA Mediated Silencing in U-266 Cells

U-266 myeloma cells (ATCC no. TIB-96), a human IgE cell line, express IgE on the cell surface and secrete IgE. Cells are cultured in RPMI 1640 medium with 2 mM L-glutamine adjusted to contain 1.5 g/L sodium bicarbonate, 4.5 g/L glucose, 10 mM HEPES, and 1.0 mM sodium pyruvate containing 15% fetal bovine serum, at densities between 1×10⁵ and 1×10⁶ cells/ml.

Cells are treated with immunoliposomes containing IgE-specific RNAi based vector conjugated to anti-human Transferrin Receptor (purchased from Biosource, Camarillo, Calif.) according to the procedure described in Example 1 above. The effect of the immunoliposome treatment on IgE expression is assessed by quantitative PCR, ELISA, flow cytometry and western blotting. The appropriate antibody concentrations are predetermined in prior experiments with antibody-liposome complexes containing CMV-EGFP expression vectors.

The effects of treatment are monitored over a period of several days, by measuring total IgE production (cells and medium) (by Western blots, ELISA, flow cytometry) and IgE mRNA (by Northern blots, QC-PCR).

b) SiRNA Mediated Silencing in HEK293T Cells

293T cells (ATCC no. CRL-11268), a human embryonic kidney cell line, were co-tranfected with plasmids containing either the cDNA corresponding to the constant region of the mouse IgE (referred to as mIgEc) or the mouse FcεRIβ subunit (referred to as mFcεRIβ), and plasmids containing shRNA sequences against these targets to determine the silencing of expression of the mIgE and mFcεRIβ mRNAs by the shRNA sequences. The cDNA for the mouse IgE cDNA (SEQ ID NO: 925) and the mouse FcεRIβ subunit (SEQ ID NO: 933) were cloned into the mammalian expression vector pCDNA3 following standard cloning procedures. The shRNA constructs containing the target sequences for the mIgE cDNA are given in SEQ ID NO: 926-929 (referred to as C1 to C4, respectively), with those for the mFcεR1β subunit being given in SEQ ID NO: 934-937 (referred to as wis444T, wis81T, wis966T and wis742T, respectively). These sequences were cloned into pSilencer plasmids (Ambion, Austin Tex.) containing a U6 promoter, following the manufacturer's instructions. The constructs consisted of the target sequence in sense orientation, a loop sequence, the complement of the target sequence and a RNA polymerase III terminator sequence, as described by the manufacturer. Exemplary constructs containing the target sequence C1 for mIgE cDNA and wis444T for mFcεR1β subunit are given in SEQ ID NO: 932 and 938, respectively. HEK293T cells were cultured in DMEM with 2 mM L-glutamine, 1.0 mM sodium pyruvate and 10% fetal bovine serum, at densities between 1×10⁵ and 1×10⁶ cells/ml. The cells were transfected using Lipofectamine 2000 (Invitrogen) following the manufacturer's instructions.

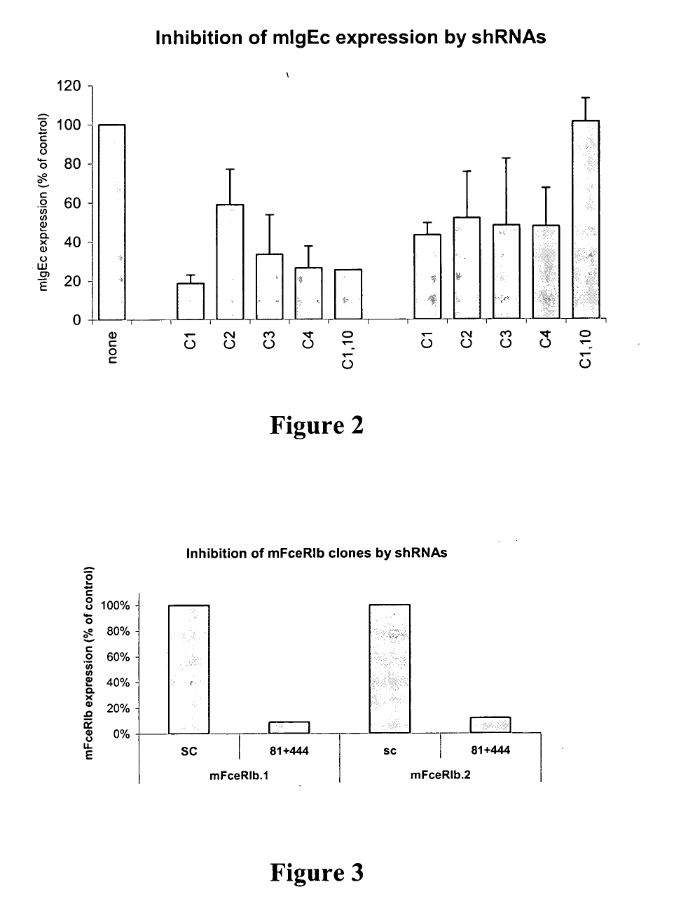

Expression of the mIgEc cDNA and the mFcεRIβ cDNA in the presence of shRNAs was measured at 24 and 48 h using Real Time PCR. FIG. 2 shows that the shRNA constructs were all found to be capable of knocking down mIgEc expression, with C1 giving the greatest knock down at 24 h (80%) and 48 h (60%). For the mFcεRIβ subunit, two constructs (wis81 and wis444) administered together effectively induced an RNAi effect, leading to greater than 90% reduction in message (FIG. 3).

The inhibitory effect of the shRNA constructs on the mouse IgE and mouse FcεRIβ subunit in 293T cells was also measured using flow cytometric methods. The cDNA corresponding to mIgEc (SEQ ID NO: 925) and mFcεRIβ (SEQ ID NO: 933) were subcloned into the pd2EGFP vector (BD Biosciences), which yields a fusion protein consisting of the gene of interest with a C-terminal destabilized EGFP moiety. Co-transfections with shRNA constructs were carried out as described above. The expression of these fusions was measured by flow cytometry: cells considered viable by dye exclusion were analyzed for EGFP expression, and shRNA activity.

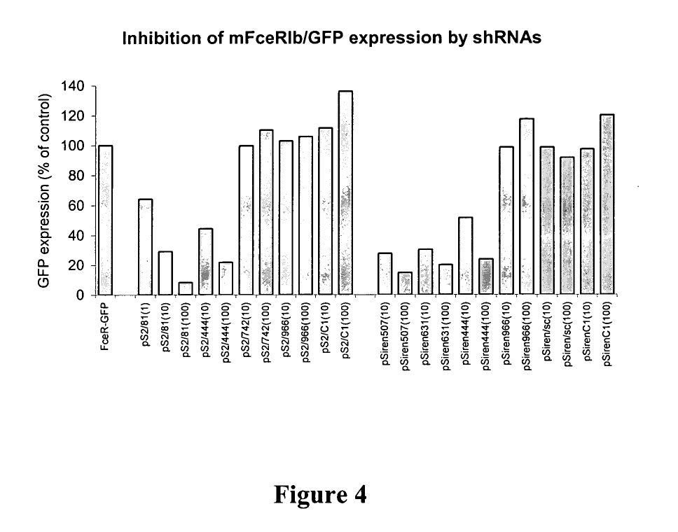

As shown in FIG. 4, the results obtained with the mFcεRIβ/GFP fusion were consistent with those obtained by RT-PCR. The shRNA constructs wis81 and wis444 reduced expression of the mFcεRIβ/GFP fusion protein by greater than 80%. In addition, constructs containing two additional mFcεRIβ target sequences, referred to as inv507 and inv631 (SEQ ID NO: 939 and 941, respectively), in the pSiren vector (BD Biosciences Clontech, San Jose Calif.) were also able to reduce fusion protein expression by at least 80% compared to the controls, which had no effect. In contrast, inhibition of the expression of the mIgEc/GFP fusion by the mIgE targeting shRNA constructs was not observed. This lack of inhibition may be due to inefficient translation of the fusion, and may be overcome by insertion of an improved Kozak consensus sequence in this clone. The full sequence of the inv507 construct (i.e. the sense, loop sequence, antisense and terminator) is provided in SEQ ID NO: 940.

c) SiRNA Mediated Silencing in Murine IGEL b4 Cells

IGEL b4 (ATCC no. TIB-141) cells are a murine IgE secreting hybridoma line. The cells are cultured between 10⁵ and 10⁶ cells/mL in Dulbecco's Modified Eagle's Medium with 4 mM L-glutamine containing 4.5 g/L glucose, 1.5 g/L sodium bicarbonate and 10% v/v foetal calf serum. IgE protein can be detected via cell surface and intracellular staining with anti-mouse IgE antibodies conjugated to fluorescent labels (eg anti-mouse IgE-PE). These cells also secrete large quantities of IgE into the culture supernatant which can be readily detected by standard sandwich ELISA (eg PharMingen OptEIA™ Mouse IgE ELISA Set).

Plasmids (for example, pSilencer 2.1) that express short hairpin RNAs targeted to IgE (IgE-shRNAs; SEQ ID NO: 926-929) or scrambled controls, were transfected into IGEL b4 cells via electroporation using a BTX ECM600 (Holliston, Mass.). Cells were co-transfected with a plasmid expressing enhanced Green Fluorescent Protein (eGFP) as a reporter. Cells were assessed for transfection and knock-down of IgE by flow cytometry (LSR, Becton Dickinson) at 24, 48 and 72 hours post-electroporation. To measure knock-down in transfected cells, IgE expression on eGFP⁺ cells was assessed and IgE expression was recorded as a mean fluorescence intensity (MFI).

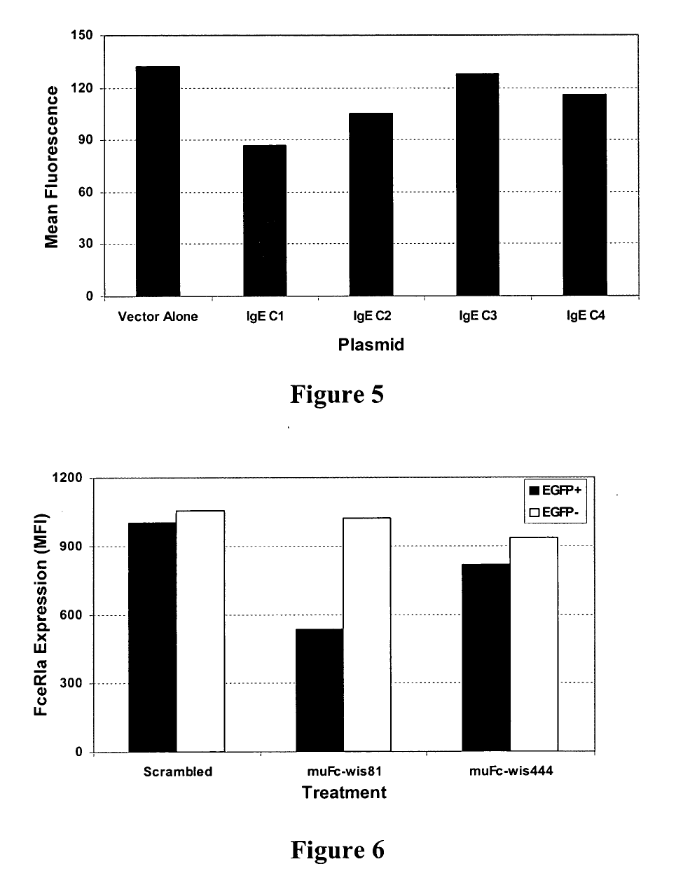

The IgE-C1 shRNA in pSilencer 2.1 induced approximately 30% knockdown of intracellular/cell surface associated IgE protein, relative to the vector alone control, as determined by flow cytometry at 48 hours (a representative example is shown in FIG. 5).

d) SiRNA Mediated Silencing in Murine MC/9 Cells

MC/9 cells (ATCC No. CRL-8306), an IL-3 dependent murine mast cell line derived from foetal liver, were cultured in Dulbecco's Modified Eagle's Medium with 4 mM L-glutamine supplemented with 4.5 g/L glucose, 1.5 g/L sodium bicarbonate, 2 mM L-glutamine, 0.05 mM 2-mercaptoethanol, 10% v/v Rat T-STIM (Becton Dickinson #354115) or 10% v/v WEHI-3 supernatant as a source of IL-3 and 10% v/v foetal calf serum. Cells were maintained at a density of 2×10⁵-2×10⁶ cells/mL. The alpha subunit of the FcεRI (high affinity IgE receptor) can be detected on the cell surface of MC/9 cells by flow cytometry using a commercially available antibody (Clone MAR-1, eBioscience #13-5898) conjugated to PE. The subunit targeted by the FcεRI constructs described herein is actually the beta subunit, but in rodents all three subunits (αβγ₂) are required for expression of FcεRI, therefore detection of the alpha subunit was used as a surrogate measure of β knockdown (there are no anti-mouse FcεRIβ antibodies commercially available).

Plasmids (for example, pSilencer 2.1) that express short hairpin RNAs targeted to the FcεRIβ (muFc-shRNAs; SEQ ID NO: 934-937), or scrambled controls, were transfected into MC/9 cells via electroporation (using a BTX ECM600). Cells were co-transfected with a plasmid expressing enhanced Green Fluorescent Protein (eGFP) as a reporter. Cells were assessed for transfection and knock-down of FcεRIα by flow cytometry (LSR, Becton Dickinson) 24, 48 and 72 hours post-electroporation. To measure knock-down in transfected cells, FcεRIα expression on eGFP⁺ cells was assessed and FcεRIα expression was recorded as a mean fluorescence intensity (MFI).

The muFc-wis81 shRNA in pSilencer 2.1 induced approximately 50% knockdown of cell surface associated FcεRIα protein, relative to a scrambled control, by flow cytometry at 48 hours (a representative example is shown in FIG. 6).

It is known that siRNA can produce nonspecific concentration-dependent effects on mammalian gene expression (Scherer and Rossi, Nature Biotechnology 21:1457-1465, 2003; Persengiev et al., RNA 10:12-18, 2004). These off-target effects can be minimized by selecting siRNAs with unique sequences, and using them at subnanomolar to nanomolar concentrations. In the above experiments, siRNA concentration is optimized for downregulation and nonspecific effects. Nonspecific effects are assessed by microarray-based expression profiling.

### EXAMPLE 7

### SiRNA-Mediated Silencing of IgE Expression in Mice

IgE production is induced in mice by immunization with ovalbumin emulsified in a Th2-driving adjuvant such as Alum. IgE production is verified by testing serum for IgE. Mice are treated with an antibody-liposome complex containing an IgH promoter- IgE-specific RNAi vector. IgE production is measured in the serum over time by ELISA.

The ability of the inventive compositions to inhibit the development of allergic immune responses is examined in a mouse model of the asthma-like allergen specific lung disease. The severity of this allergic disease is reflected in the large numbers of eosinophils that accumulate in the airways and the levels of IgE detected in the serum.

BALB/cByJ mice are given 10 μg ovalbumin in 1 mg alum adjuvant by the intraperitoneal route at time 0 and 7 days, and subsequently given 100 μg ovalbumin in 50 μl phosphate buffered saline (PBS) by the intranasal route on days 14 and 18. The mice accumulate eosinophils in their airways as detected by washing the airways of the euthanased mice with saline, collecting the washings (broncheolar lavage or BAL), and counting the numbers of eosinophils. The inventive compositions are administered to the mice intravenously at various times before intranasal challenge with ovalbumin, and the serum IgE levels and percentage of eosinophils in BAL cells collected three days after challenge with ovalbumin, is determined and compared to control mice.

Eosinophils are blood cells that are prominent in the airways in allergic asthma. The secreted products of eosinophils contribute to the swelling and inflammation of the mucosal linings of the airways in allergic asthma. Reduction of the accumulation of lung eosinophils upon treatment with the inventive compositions indicates that the compositions may be useful in reducing inflammation associated with eosinophilia in the airways, nasal mucosal and upper respiratory tract, and may therefore reduce the severity of asthma and diseases that involve similar immune abnormalities, such as allergic rhinitis, atopic dermatitis and eczema.

### EXAMPLE 8

### Suppression of RNAi

The therapeutic use of siRNA to knockdown IgE production in IgE-mediated diseases in humans and non-human animals may require rapid reversal when antigen (allergen) is no longer present. Suppressor proteins from plant viruses are capable of reversing silencing in plant tissues where it is established, and preventing initiation of silencing in new tissues. Plant virus genes encoding suppressor proteins include HC-Pro (Tobacco etch v irus), P25 (Potato virus X ), 2b (Cucumber mosaic virus), Turnip crinkle virus coat protein, and p19 (Cymbidium ringspot virus).

Some plant virus silencing suppressor proteins are functional when expressed in cultured Drosophila cells (Reavy and MacFarlane. Scottish Crop Research Institute (SCRI) Annual Report 1000/2001, pp. 120-123). The B2 gene of the flock house virus (FHV), a nodavirus that infects vertebrate and invertebrate hosts, initiates and is a target of RNA silencing in plants and Drosophila cells (Li et al., Science 196:1319-21, 2002). Vaccinia virus and human influenza A, B and C viruses each encode viral suppressors (E3L and NSI) which bind dsRNA and inhibit the mammalian IFN-regulated innate antiviral response (Li et al., Proc. Natl. Acad. Sci. USA 101:1350-1355, 2004).

The effectiveness of these viral suppressors of RNAi may be evaluated as described above in Examples 6 and 7.

SEQ ID NO: 1-980 are set out in the attached Sequence Listing. The codes for polynucleotide and polypeptide sequences used in the attached Sequence Listing confirm to WIPO Standard ST.25 (1988), Appendix 2.

All references cited herein, including patent references and non-patent references, are hereby incorporated by reference in their entireties.

1. A composition comprising:
(a) a small interfering nucleic acid molecule (siNA) that is capable of reducing expression of a target gene that is active in a IgE-mediated disorder; and
(b) a binding agent that specifically binds to a target antigen expressed on the surface of the cell,
wherein binding of the binding agent to the target antigen results in internalization of the binding agent and the siNA into the target cell followed by release of the siNA.

2. A composition comprising:
(a) a genetic construct that expresses a small interfering nucleic acid molecule (siNA) that is capable of reducing expression of a target gene that is active in a IgE-mediated disorder; and
(b) a binding agent that specifically binds to a target antigen expressed on the surface of the cell,
wherein binding of the binding agent to the target antigen results in internalization of the binding agent and the genetic construct into the target cell followed by release of the genetic construct.

3. The composition of claim 2, wherein the siNA is under the control of a cell-specific promoter.

4. The composition of claim 2, wherein the siNA is under the control of a promoter selected from the group consisting of: RNA polymerase III-dependent promoters; and RNA polymerase II-dependent promoters.

5. The composition of claim 2, wherein the genetic construct is packaged within a viral vector.

6. The composition of claim 5, wherein the binding agent is a viral capsid protein.

7. The composition of any one of claims 1 and 2, wherein the target gene is selected from the group consisting of: IgE; FcεRI; and STAT6.

8. The composition of claim 7, wherein the target gene is IgE and the target cell is a B cell.

9. The composition of claim 8, wherein the target antigen is selected from the group consisting of: CD19 and CD22.

10. The composition of claim 7, wherein the target gene is FcεRI and the target cell is selected from the group consisting of: mast cells and basophils.

11. The composition of claim 10, wherein the target antigen is selected from the group consisting of: FcεR1 and CXCR4.

12. The composition of any one of claims 1 and 2, wherein the binding agent is selected from the group consisting of: antibodies; antigen-binding fragments thereof; small molecules; hormones; cytokines; ligands; peptides; and viruses.

13. The composition of claim 12, wherein the binding agent is selected from the group consisting of: anti-human CD19 antibodies; anti-murine CD19 antibodies; anti-human CD22 antibodies; anti-murine CD22; anti-human FcεR1 antibodies; anti-murine FcεRI antibodies; anti-human CXCR4 antibodies; anti-murine CXCR4 antibodies; and antigen-binding fragments thereof.

14. The composition of claim 12, wherein the binding agent specifically binds to CXCR4 and is selected from the group consisting of: CXCL12; peptides; and small molecules.

15. The composition of claim 14, wherein the binding agent is selected from the group consisting of: 2,2′[4,4′-[[aminocarbonyl]amino]bis[N,4′-di[pyrrole-2-carboxamide-1,1′-dimethyl]]-6,8 napthalene disulfonic acid] hexasodium salt; [Tyr^{5,12},Lys⁷]-polyphemusin II; N-α-acetyl-nona-d-arginine (Arg) amide; and the CXCR4 antagonists AMD3100 and AMD070.

16. The composition of claim 2, wherein the genetic construct is connected to the binding agent by means of a streptavidin-biotin linkage.

17. The composition of any one of claims 1 and 2, wherein the siNA or genetic construct is encapsulated in a liposome, and the liposome is attached to the binding agent.

18. The composition of claim 17, wherein the liposome is a pegylated liposome.

19. The composition of claim 17, wherein the liposome is attached to the binding agent by means of a maleimide linker.

20. The composition of any one of claims 1 and 2, wherein the siNA or genetic construct is attached to a lipid or polymer carrier.

21. The composition of any one of claims 1 and 2, wherein the siNA is targeted against a region of the target gene selected from the group consisting of: 5′ untranslated regions; coding regions; 3′ untranslated regions; and promoter regions.

22. The composition of claim 19, wherein the siNA is targeted against a sequence selected from the group consisting of: an mRNA molecule that encodes IgE or a portion thereof; an IgE promoter sequence; an mRNA molecule that encodes the high affinity FcεR1 receptor alpha subunit; an mRNA molecule that encodes the high affinity FcεR1 receptor beta subunit; an FcεR1 receptor alpha subunit promoter sequence; an FcεR1 receptor beta subunit promoter sequence; the 3-prime UTR of the FcεR1 receptor beta subunit; and IgE epsilon domains 1-4.

23. The composition of any one of claims 1 and 2, wherein the siNA is between 19 to 30 nucleotides in length,

24. The composition of claim 20, wherein the siNA is between 19-25 nucleotides in length.

25. The composition of any one of claims 1 and 2, wherein the siNA comprises an antisense strand that is complementary to a mRNA sequence corresponding to a region of the target gene.

26. The composition of any one of claims 1 and 2 wherein the siNA is selected from the group consisting of: dsRNA molecules and shRNA molecules.

27. A method for the treatment of an IgE-mediated disorder in a patient, comprising administering to the patient a composition of any one of claims 1 and 2.

28. The method of claim 24, wherein the disorder is selected from the group consisting of: allergic rhinitis; asthma; anaphylaxis; urticaria; atopic dermatitis; food allergies; diseases that benefit from the reduction of eosinophilia in the tissues of the respiratory system; and disorders characterized by a hypersensitivity immune reaction.


SEQUENCE LISTING (980 sequences)

SEQ ID NO: 1 - DNA, 2150 bp, Human
aacagagttatcgaggaggggtggctgctagccattctgagctcatcccaggcccctgggtctccgggtcactcccattctgactgatcaatcaccaaaagccaaggagggcccggcacccagcccagggcacagctgagtctgcgtccagcccaacaccagcccacggcctcactccccagcctcggtctgacccttctagccctgagatccaagtgcccggcatcccgtccccaagcctcacccagaccttctttcccttcaccctcccctcctgccacccatcctctgcccccatcccttcacccacccctcctgccatctatcctcagcccccatcccttcacccacccctcctgccatccatccacagcccccatcccttcaccaccctcctgccatctatacctcagcccccatcccttcacccacccctcctgccatctatcctcagcccccatcccttcacccacccctcctgccatccacctgcagcccccatttctgcagcctgaaggcacagggaagccctctgaggccaggccacaggacagttgagggcttcgggcccctggagtgggtagagggctcccgcagccagagaaggggtttcctgcagggacagcagcgtccgacctcgtccctatgacctcgtccagcctctggcttcagcagtcctacttctttctctctgggtttcttagtaaagatccttttcacaaaccccacgtctccagagggctctgtgggaggtgtgtgtggccctggagtggctggcgggaggcaggacccctgaggacgtgcaggaggagccagaggggaaggaagggcaggggccattctggggtcctgagagccaaagtgcccaagcagtggttcccatcaatcacccctcagcaccccagagactccccagctcggccctgtcatacctggtctgctggtctccagcccagactcatgcctccttctcaggtaaagtcaaagcaacaatcaacatcgaccctttgtggcaaaaccagaagaggccatgctgtggtcagcacaggaggccaagaatgcagctgcacagtcaccaaggtggaggctggtgggggcccccaggaccactggccacacacccttggatccagcacccactgagcacagacaaagctgactccccagcccacagccgcccaccagcctggacctcctgaagagctgagaggtgacccaggagcaggctggggccccagagccatcggggccacaggtgggcttgacccacaccagtcctcagggcaaatgtgcagcctccctggctggagaggcctgcttacaagggtcttccacaacatcctaaaatgggagagaacgcatgataggcatgacaaagggcagggttccctcatatacaaagagcactcagagatcactaagaagaaaggtgagcaataggttcttcatggaaaggaaaaacccagtggccgataatataaagaaaccattttaaaatgagaaaagtgaaaggaaggaggaggggatgaagggggtggaggtgcttcttggccctgactgagggcacgtaaatcattaagaccactttttagggccagttggcaaaatgccccaatgcaggagagtcctctcacagtgatggccccacagggagagcctcgctctgtgccaggagagccacgtacactggagtctggcacaaagcggaatgaagcaagcctgcctcagtctgacagcggcacctgggccctccgccctgtcctctgctgcgacctcggagcagccgggcacgggggagaaggtgaggagcgacggctccctacagggtagaaaaccccaaagcgggcagcgctgaggccgactgggtccaaacaacaccagaggaggggcgtcgcaggacagggccaaggccacagccgctgcccagctggggtcacacactccctcattcacagacacacatgcaggcacacactcgcacatgcaaaaacacacaggttcacacacacagactcaacctctcatacacatttgcactcacacacaccaatcatgcactcacacaggctgacacgctcaggcacgctcataccaacacacacacagtctcaggaagccaggccctccatccaccccatcaccagcccagg

SEQ ID NO: 2 - DNA, 2062 bp, Human
tatattacacatatatattttgatatatatatatctccaaaccatctaaatatcaagtattttttaatccatctaagagctgaaattgctgaaaaaactactccctccaaaagctgtagagacaggcacatccacagtcacagcagagacttgctgacttggaagagaagctcctggagacacattggtgagaacatttacctggtgattatgctgaatgtctggaggacaaatgtggactagggggagggtgagcactcctagaggctgtacaccccacacttgtgtggacttgccctccagggccttcaggttctcgtggaagcaattaaaatagattccctatagccatgaactggggaggagtaatcacggagaaaagatgcacaaaaagacttttctagaaagctcatccaagggaaggtattctccaaaatcttagtttatgtgggggaaggaaatacttccaaattacagacccctcctcctcagccttcctctatcacgcaaatgataaaattagccaagaggagtcagattcaaggcggtagccctgggtgcagcatctgcagaagggaggaaagagagaaaatcagctgtatcactggagattccttgtgaaggtcactgctcaggagaagagggcaaccaaaccagggagagtcaactgtaagaacataccatgctcccctgccccacacattacctcctcaacagcatcattaatatggattaaagagggcagtgtgattgctttagatctgtttgagaaagaaagtcacatactgaggcctagggtcagggtcggcggcacttcccgtgagtaagatactacgaagaaggaaaaattaggggtccataactgtgaaaatcagccacagtgtgtgtgagaatgtttgtgtttgtgtttctgtgtgtttgagtaggagttattggaacagcggacgtggagtgagctttaatccacatccatctgcagcttcaggtattctcagatgcagtattcatctgcaagagccgaaatgagaaaagagccacctccaacccccccagagttttagcctccctttgtttccagtgatccagtgcatctagacctccaggaagtggactccctggtgattttagcgattcttctcttggagccaccctgaagaggacattgggtttccaaaggcccattcactatttcaagaagtggtgccatcagctcatgttgtcactgaaggagcattctgagccagggcacagtcacttcctagtgagctacagaggctgagagaaaaatgctctgtgagacccaatgggaagctccctgcagtgcaaggtctgggtggcagggagcgctagggcctcgcccagcacaggctgcagccctggagcaggtgcaagggaggctggggaggggttcctcccagggtctgatgtcttccttttctcggacaaacatgctttaataagttaaacaagactttagtaaagactattgatgtgtctttgtgtctttcagtatacagttctatttgtaggatttatctaacctaacaagtcaatgagaatcacatgtaaaaggagaaatttctaggattttcagatatcttaataggtaggagatggagaaaagggatggttttattaattcagtgcttgccaatcttaacagagacagtagtaagacatgcagaaagcaaagcccagaaaagtatgaaggtgtcaaagtgccatttaagtatgggttcacttggaggaccatgttctgcgggaacttgttttcagcagacaatctattttagcagagttctgggcatacaaggggacacacatcattaaacaaggattgggacagggacttcagcgtcccactgttgcatggcccataaattatgtgtgttctctttctcatcttggatcaagtctagagctatgaaatagtatccctcatgaatatgcaaataacctgagatttactgaagtaaatacagatctgtcctgtgccctgagagcatcacccagcaaccacatctgtcctctagagaatcccctgagagctccgttcctcacc

SEQ ID NO: 3 - DNA, 1198 bp, Human
tactaagagtctccagcatcctccacctgtctaccaccgagcatgggcctatatttgaagccttagatctctccagcacagtaagcaccaggagtccatgaagaagatggctcctgccatggaatcccctactctactgtgtgtagccttactgttcttcgctccagatggcgtgttagcagtccctcagaaacctaaggtctccttgaaccctccatggaatagaatatttaaaggagagaatgtgactcttacatgtaatgggaacaatttctttgaagtcagttccaccaaatggttccacaatggcagcctttcagaagagacaaattcaagtttgaatattgtgaatgccaaatttgaagacagtggagaatacaaatgtcagcaccaacaagttaatgagagtgaacctgtgtacctggaagtcttcagtgactggctgctccttcaggcctctgctgaggtggtgatggagggccagcccctcttcctcaggtgccatggttggaggaactgggatgtgtacaaggtgatctattataaggatggtgaagctctcaagtactggtatgagaaccacaacatctccattacaaatgccacagttgaagacagtggaacctactactgtacgggcaaagtgtggcagctggactatgagtctgagcccctcaacattactgtaataaaagctccgcgtgagaagtactggctacaattttttatcccattgttggtggtgattctgtttgctgtggacacaggattatttatctcaactcagcagcaggtcacatttctcttgaagattaagagaaccaggaaaggcttcagacttctgaacccacatcctaagccaaaccccaaaaacaactgatataattactcaagaaatatttgcaacattagtttttttccagcatcagcaattgctactcaattgtcaaacacagcttgcaatatacatagaaacgtctgtgctcaaggatttatagaaatgcttcattaaactgagtgaaactggttaagtggcatgtaatagtaagtgctcaattaacattggttgaataaatgagagaatgaatagattcatttattagcatttgtaaaagagatgttcaatttcaataaaataaatataaaaccatgtaacagaatgcttctgagtaaaaaaaaaaaaaaaaaaaaaaaaaaa

SEQ ID NO: 4 - DNA, 894 bp, Human
tctcaatataataatattctttattcctggacagctcggttaatgaaaaaatggacacagaaagtaataggagagcaaatcttgctctcccacaggagccttccagtgtgcctgcatttgaagtcttggaaatatctccccaggaagtatcttcaggcagactattgaagtcggcctcatccccaccactgcatacatggctgacagttttgaaaaaagagcaggagttcctgggggtaacacaaattctgactgctatgatatgcctttgttttggaacagttgtctgctctgtacttgatatttcacacattgagggagacattttttcatcatttaaagcaggttatccattctggggagccatatttttttctatttctggaatgttgtcaattatatctgaaaggagaaatgcaacatatctggtgagaggaagcctgggagcaaacactgccagcagcatagctgggggaacgggaattaccatcctgatcatcaacctgaagaagagcttggcctatatccacatccacagttgccagaaattttttgagaccaagtgctttatggcttccttttccactgaaattgtagtgatgatgctgtttctcaccattctgggacttggtagtgctgtgtcactcacaatctgtggagctggggaagaactcaaaggaaacaaggttccagaggatcgtgtttatgaagaattaaacatatattcagctacttacagtgagttggaagacccaggggaaatgtctcctcccattgatttataagaatcacgtgtccagaacactctgattcacagccaaggatccagaaggccaaggtcttgttaaggggctactggaaaaatttctattctctccacagcctgctggtttt

SEQ ID NO: 5 - DNA, 14655 bp, Human
ataatgtattatatatgtatataggcatatttatatatgtatgtatatttatatataaatataaaattatatattatatatgtgtgcagatagataaagacttgcctctgaggaaggaaaggatgatttattaggttgtaacatcttgatataataatcaatgaattaattgctcatttgcctctctaataaatacatattaaaaatctaagcatgctctctgctgtgctgagtatcagataaagagcagtgaactaaactaatatagtcattgacttcaatgaggatgtgataaggagctagaccaggaaagatgcaattttcatatattgtgatacatgctatgataaggtaagaatggagtaatatgggagcaataagtaatcttctggcatgcattttaatatatgaattgcttacattcaccagaaagtgaaagccatatagaaatgactggtctggtacacttactgttaaaaatgaatacattcacatactaagttattttgtaagtttgagtaatgtctcagagactggtcagctccagtgagtttaccattgagatctagggcttgcatggtagagacacagaccctggatcatgggggctacaccaagatttttctaattaccaaattcatcggactattcttggttctttccctacttcataactgaaacatttaacactattaatcactctccctcttgaaatgctcttctgccttggttttgtgacactactatctcctggtgtttctgttagagtttactggatgtggtcatttagtgtgtagtgtgcatctgtcagtctgcactgcagaagcgatggtgaatgtcatttactctggtaattcagttaacctgggttatcaacagagctctactgcactcttatcatttcagtatgtgattaacctaaatattttaaccatccactaactctatctcatcattctaatttaattctctctgtgttactacctggtatattttgttcaataatttgttcactttttaattgactttctccccatgatggactataataatccttgagggcacctgttctctcatcctttccttggtcctgtatcttcaactaaagcagcagcagaaacaacaacaggagcctcacaatttagtactgcattttccactaattaattgcttagatatataattctcacaaaacattctgaggggttcgggcttctcaatccctaaaatgaagaaatttaaattggttatgactatgatcctttcctttttaacatttttcctatactttaagacttatgataaaaactcattaggaaactagaaataacatgtcttccccaagtcacttctaacaaatatcccatggttatcgtcagtcctgccatctttccactattctgcttccaactcctttctttctttgacttccactttcaaatctatcacgacctcctgctgctctttcttttctgccagctcagagtcagaggcatagaaggcaaggttactgtagatcttgaactgtcaacctggcaggcaagataggagagtgaaggactcatcaactagccagtagcagtcactgctctttagaagccaccttaggaccaaattgttcaagtaactgaactgttatcttcctgaactcctgcaattctaattgccagaaaacatctagatgttgggcctgcagaggaccttagcacacttgtagtacaatctttccatttcatacacacaactgtgttgaaagaagtctcaacttgtgaggtgtaaacatgaaatcattatttttgaaattactactactgctgtggacccattcttcttgggcagtagcctcccttaatgacttcttagactaattccataaccttttaatcactttatgctactaaaacagtctcattggagttattaggcacaatcaaatctaattaattttgattttataaaattaatcaatttaattttatcttctgttcaaaattcttttcagtctccagcatcctccacctgtctaccaccgagcatggggtaagtttatggctccagtggcttgttatgtgttgggaggtaaagggtgatggccacactactcacatccctccctcttccagatatcagccccttccttgagagtaggaagaagaaagcaactctccatgctgtattagctgtcggctccttgttttgttttaactgctattcctgtgtgaacactacctcataagtggaggtcagattgtggtagtgtaacctttttgctagttgtcaccatttctctagcattattcattgttctttctcatttgcagtgattcaaatataggtgctctcatgggaatacctttgtaggttctccagaagaacctatgtcccatgcatctgcacagcaaaaagaatgacctattaacttatggttcaatcaattctactttcctctgctctcattgcaggccgcccactccagagcctcttgttattagagttcccctcacccagagagtgtcccctttgagggagagggacaatatggcttaagttcttgtcactttcctttatgcccacctaactcaggggtacttaagtaggtgccacacaatggaggctaactctagcaccctctctctcaggtccattacttctaacctgccttttcctgcaaaccctagcagtgtcagattctccatcagtaccctagcagtgtcagattctccatcagtatcttgcctcataaattttaagtcaaaagatgaccacaaactctcaacttattggagggaataattaagttatcccaaatgttgtggccaaagtgctggagcaccttgttgaatgcctttcttcctcatgtttaagatccctaccattcagacctctaagcttcaaatcctggctttgccattcactagttgtataatctaaacctccctacctcattaaaaacaaggatgctgatagtacctgtttcataggattcctgtgagtattaaataggataaagcacaatgcctgaatacagtcagtgttcaaaaaatattaacagttacctgtgcttatatttttattagaacacatcctagtttctccattattcttcctgcttatggaaatcaagtttttaataaagtcattgtggtttggatgaactcccttctgcatttttgtattctacgcagtattccacatattgctatgcacatggcaactgcttaacaaatcaagaaatacattgcagtttctctgatataatgcatttttctgtaatgcaaattagtctatatgctataggcaagccaaggaataatgtcaatataacattgaagttggttgaaacaagatttttttctagcaagttaatattttgaactaccaaggaacagctaacacaaatcttactagcacagtagaacatggcaaggaatagaatattctgcacagtaactattcacttcaaggttttctcttggcttagttttaccacaagtgtatcttgagcatgtttgttctctggttttcccctgcttttgtgtattttctctttgtcatcttcttccaccaacctttgaaagtgatcaagtcctagatttactgttaattttatttattacaaatttaacatatctttttaactgcatagttcatttggtagaaatactaatgtttatgttactgctctgatggtaggttttcatgatctgtaacagtctttttttcctctcaaagacttgttagttttagttatttttgcagaatgcaaaacaatttaaaaaaataaatgtatgttacatgttatggcagaaacacctgtgttatgttcaagaaagatttaaattgaatgagtgaaagccacttctctcttcttgattatgcaagggcttaacttcttcaattatttaacttatacttcccttattccctctctgcttactgaattattcctgttagctaacaaatacaatccagcatcttatataaaacaagaatcctcccttaaccccacatcttcctataccaccatcccacagcaaaacttctcagaatattgtccgcacatgttgcctatgcttcctcaattcctattcacttttttagcccacttcagtttgctttttacacatattactccactgccattgctaaagtcccaaaaacttccatattgctagattcagaggataattgtattctctattttacttcacctctcaacaagattcaatgtaattaacgatttcctctagaattgtactctcttcttggttttagtgacataattccctgtgagttttcttaccttatcagctgttttctctagcctctgttctgggtcttctgatactcatcttcaagatattgaaatttcctagagttctgctctgagactttctctgttgcttaatttctttttctctctcttcagctgacctcatcttactcagtggctttaaagtatgccagtgattctctactttctctactttatatctctaatatagacatttctactggtctctcaacattttttttgtaactgcctatttagcacagactcttagatacaataaagatgcttcaaactaaaacacttcccaaccggaacacttggttgatttctcccccaattctagtacttcccttaaatatagtacttccccagtcttcaccatgtaattactgtctacccagttattacagccagaaatttaagaatcatattttatactttctgtgccctccaatctgtcagtaagtcccattaatatatatacctcaaacccatcaacttttctttatctatactaccacttctctagactatctatcatcatattttgactcttaagcatttcccaatatctgctcaaaaaacctacagattacatgacgacatacctatctatatatagatagatcggtagacgttgagagaatgtgggtgtatacacatatactatacatatacatgtctatggtttgagtgtttatcccctccaaacttcgtgatgaaacttaatttccagtatggcagtattgagaagcaggacctgtaagaggtaattggttcatacagattctgccctcatgaatggattaacctaattatgaattaatagattaattgaataatgggttatcatgggagtgagactggtggctctaaaagaagtggaagagagactagagcttacaggctcaagccccccttgccatgtgaatctcggtgccacctcgggaatctgcagagagtccccattaagaaagaatgtcctcaccagatagaccccttgaccttgaacttcacagtctccagaactttaagaaataaatttattttctttataaatttcagtctcaggtattcagttataaccacagaaaatagactaagacatacatgaagagccatcaaactgtattaagcaggggaatatatatacatacacactgtgtgtgtgtgtgtgtgtgtgtgtgtgtgtgtatgtgcattcttttgcttcacacagtccggtggctctttcttagctttcaaataaaatcccaacactttttcatgttcttccaggccctcatgatctggcccctatgtgtatctccaatctcgttttatgccattggccttctactcgctatgcttcagccctaatgtccttctcttaatttcttttttttaaattttattattattatactttaagttttagggtacatatgcccaatgtgcaggttagttacatatgtatacatgtgacatgctggtgtgccgcacccattaactcgtcatttagcattaggtatatctcctaaagctatccctcccccctccccccaccccacaacagtccccagagtgtgatgttccccttcctgtgtccatgtgttctcattgttcaattcccatctatgagtgagaatatgcggtgtttggttttttgttcttgtgatagtttactgagaatgatgatttccaatttcatccatgtccctacaaaggacatgaactcatcactttttatggctgcatagtattccatggtgtatatgtgccacattttcttaatccagtctatcactgttggacatttgcaacctactcatctgacaaagggctaatatccagaatctacaatgaactcaaacaaatttacaagaaaaaaaacaaacaaccccatcaaaaagtgggcgaaggacatgaacagacacttctcaaaagaagacatttatgcagccaaaaaacacatgaaaaaatgctcaccatcactggccatcagagaaatgcaaatcaaaaccacaatgagataccatctcacaccagttagaatggcaaccattaaaaagtcaggaaacaacaggtgctggagaggatgtggagaaataggaacacttttacactgttggtaggatggtaaactagttcaacccttgtggaagtcagtgtggccattcctcagggatctagaactagaaataccatttgacccagccatcccattactgggtatatacgcaaaggactataaatcatgctgctataaagacacatgcacacgtatgtttattgcggcactattcacaatagcaaagacttggaaccttctcttaatttctacaattctccaaagtctttctttttatggggactttcaacttcccactgcttgaaaattcttgttccagcctttgtcttgtttcatcctatctcgtactttaagtctctacttaaaagtcatttttcatcaaatggcctttcttaaccctatttaaaacagattgtccttgtacgtctatggagcaacattatttactttttacaaacgttagtcacaagctgtcactgttctgtttacttatttattttctgttttctccaatagaaagtcttctccccaagggaaaagatcttgtctgcttattatctctcttgtatcaccagcaaatgacagagtgcttggaatataaatacttgttgaacaaagaaatgaaaatatatgatgaagaatgttcctatgctgaaactaaatttttgtgttagttttagtgacagaaagatcaagtaacaggtctgtatccatatttaagccatttctttatttgtccataataattcaagctcagagattgcctgtttggaaaagtatattataaaaaacactattaataaacttcaagacatagtttgctcattctgaaaaattagaaaatataattagccaaaattctaccagcaaactatgatcaaatgctaactttaatgagatagcccttgataattctgaagttatcttttttctcttaaagataaattggcatcatgagattattgaaatatatggttgattagtataaattcaatttaggaattgagaatacaaaaaattatttattttacattctctgctatagacttctaattaactcttgccccttgggtcatgcagagaagattttcttcatatatctttctgcaacgtgactgtgctattacctatgataaagagggttgttctgtgtttggcagcctattctagggtgggccttgtgtcagttcctttttttgtccacctgtctctgaacaacataaaaccctcattcataggtgcagcatgacttaatttgtcggtcactagatgcagttgtaaattgcaaacaaaaaaatggaaatgcagatgttgaaataaagatgcaatttttggttatgaaatggtcatctgaatgatccatatttatattcatttatttattatcaatgaaaaacgtacaaattttctaattaaaaaacttacaaattcctaattataataaacactattctacgggaatggccaccaaaatgtcccaaattttatgtcactgaatctatttctaatattcggtctaatagtctaatcacagacactcaatattcatttgttttcttgaacctctatgataatctgcaagtgtttcccgttttggaagagacaaaggaaaaagctgtgatgaatatcactggaaactgcggatcagggcccctattccatgtttctctggcccttttttcttcttcagaatttagcagtggttttcaatcctatcagatcacatttgatagcatgcatttattcctgtgtttgtaaggcataaacctgtaaccacactacaaatagtgagaatattgctgtgaaactgcatactctatgcactcccaacaatcaataatagaaaataagtggtgctcaatgatgctagagaaaagaataaattatatttctagtttgtctataaaaatgatattacaatatctctgtctgaggaagaggagatcaaataacatgtaataacaatattttgaaaaggaagaggatagtggaatatctgtttactttctgatgaattttataatgtttatgtcatttgtcagctggtatatttttaaccttttgtgattaactttcttgtcctaaatagtaattttaatatctaatattgtattcaaaaatttctactggattttttaaggaagcccccttaaactgtttaagtttcagggcctcaaactggatctacctctgctacttacacacacatatattttttaatcagtaaaattcccagttttaatataaagaagaaataaagggaaataacattataataggaaaatatgtccttcatgaatatgagtgctacaaaaacagactgatatgagaataaggtattggtgacacaaatatcatgaacagtgtagctacaggtgatctgattttctgaaacagtgaaaaattcaaggatgtttttccagtaaaagccaaggataatctttcctcaatatacatggtagtttcttttctgtaaaatacagtatgtatgagaaatgcaaaagatgcttttgtgtttttcacagtcgtgtttaaggctcagataattataaaatatctggctgcacattgaaaagatgtgcagaatcaggccaattatttagtatgcaaggctgccttcagtattgcagggcatttgtttgcctgctaatgctacaaatgtctcctgtatactcatcattgaaacaagcaaccctcaatacacatacatacattctcatgcataaacttacatttcctaaaggccttttgtaggtggtaacacagagtatgatgagaaagtttcagaatgcgtcctgcccctcccactgttcccatcctcactcataccctctttcacgcactagaaacacacgggtcacagaactgtttagccagagtcctgaaaaattaggtcttcaaaagttgcctattaaaatgcaaatgtcctggtctgggctaagatctctcatccttgtctgttaacggcctaaaaggattcctttaagagatttctctttccgttttgaaactgtgtaattctgtaaggaagcataaaataaacctggcctacactacattcggcttgagggctgactctgcacaatggggatttctctgtctcgtaccacacctgaaaatgatcttcaaacttcctaacgccacttctgcagacatgcgtgtgttggaggtgtgggtgtggttatgtgtgtgatgatgtctgtctcattgacatgattactgctgtgtgatcctggctgcatcctgtgagaacaaggttttgccttctggacccaccagtggccttccaggttcttcatcctcaggacagaccaggaggatgggaaaggaggctctctctctgctgctctcaagtctaaccaaaggcttcttgcaaaataattatagtattcatgaaaactagtatatgttctgttctaaccacctcaccacattaactcattttatgctgataaccacccttaaggtaggtactattactatctccattttactggtgagaaggctgaggcaaaaagagtttgagtggtcacagccgagactcaaactttagagcttgttcctagcctgaactcttaattaatcttgcacttaatttctttaaattatctatggctaaacttttatttttattcttttaatttttaaaaatttagtgtgatattctggtattttttttagctgggtatggtggcacatccccataatcccagcttcttgggaggatgaggtgggagggatcacttgagcctggcagtccgagactagcttgggcagcatagtaaaacccagtctcaaaaaaaattttttttaaagaaggaaaacttttttatactacaactttccttaaggatcttcctcagtgggcttataatattgttctccccattatgaagaaaatgtcagctcacttaacatctgtatctgttggaagacatgaaggtaagtttgaagccctttgggcttcttggctgaaatatactatttataaggtagaggtcatgatcaatttgtttgagtccaccttatggttcagattgtacccttaactacaagacaccacaaagatgcttcatcggcaaagtgtgagtggctgattacatatcttgaagacaggcagcttgatatctagatgcccacacctatgcactataaaagagtgtagattatgctcagtgatacgccttggatgttttctcccctccaaatctcatgttgaaatgtggtccccaatgttggaggtggggcctagttggaagtgtttgggtcatggaggcagatctcttataaatggcttggtgccttccccatggtaatgagttcacatgagagctggttgtttaaaggaaactagaacctcccttttcactctcttctttagtagtcctgggagtttccatttatttcctttggatttattacttttagacccattcaggtcaagacaagaagtaaaaaatattaaagtcactgactttcttgtgaaaatctaacaggcagtcagcatctgctattgatgctctttcttgagcagaaactcctgttcagggtagcaaaagaaagtaaatgcagctgatatcctcctggccctaatatagaggaaaaaggaaagaatccctctctcctttcttctttttaggcatgtccatgaaatatacatagctaacataccaacagtaccaacattgtgtggctgtgagttgagatgtcatggcctgaattaagaaagggaacttatagaacagaaagttggtttagatcagtgatttcctatttgtgggttatggaacccttgaatccaaggaatttctgcatgaggactatgaatccatgtgcacttaatcacaaagtgtagactcaatgaataaagacatctttatctatctatctgtcaatcaatcatctatctatctggtcatgagcatgtgctcttaagacatattgccaggttcaaattctaattcctttacatagcaactgtgtgccctgggacaagttacttaacctctcatgctttgatttccttattcttaactacatagagttggtagtaggaggaaaatagataggacatgttgcatagttagccttgtatttgatcctcagggagagatcaacatttgttaggtacataaatatcacgagcagacaggcattttgcactttcatcgtttcatattcaattttatatggacctcaccttgttttagagaactttgagagtgagaaggaagccagcagattgatttggtttttctgcttcccagttttggagaaggacataaaggctaacacactgtataggctgagccttgatcctccctctgccaggcacgtcccgaaacacaaaagttcctgcccaaagcctctcacgctggatccagttgcaggggcacctcacagtcctcaggccctgtagctcatgtacagccatttgtttcatttttccactgctacaaaatgaaatatagattaaggctcccaacatcaaataacaggtccaagtttgggcatagtgatactctttgtgtattgagcattcctgtacccagaatcagaagcaagaactgaaatttagacacgccctattgatttcagtgaacgtgtgttcctgatgtggacaggctgggtatgagaaattagtagcagcccactccacccagataatggatatgaaacttgaaggacttttttttttatatataagataaaaaacctgtggcattcaactgtgtatcagcccgttcttccagggaggaatagagctcttgtttaatgttagtgttaatgtgttccttctgtgttcttgttctgttcttttgggtacctgtagttatatgctatggaaattagggtatggctttttctttccgatgctgttaatggtttgcttttttggggtgggggtgggctgggaagatgggtagtttggttgctttttaggctttaatgagctgcttttattggactgctacatttttgctgttactgctaccaaagcacttcctttatttcaataccgggcacttaagcaaaagctttagaatgataaatccaaagtttcagaaagctttagaaatgaaagtggcctccctaactccaaagcttatactcttcgaactaggaatagtacctctcgatgagccctgctccatctcccatctaagtaggagctatttagataacgacttcagaggaagcctttaaatagactatttgagtcatggtaaaatgactctctctgagggacaatttgattgtaggttcactgtcctaggaaaaaggaacccaaatttcccctggcaaagtctcaacaaactggtaaagtagaatctttccccgtctcttccagggcttagggcttcaaatattgccctctgacagcaccttaaaaactacatcttccctatttcctcctccttctcttctccaatcctatccaacaactttctgcattctgttctttcctgattatgctctcttctttgtgactgtggagaggattgagattgcgtagatatattaaactagcataagatgttgtttatcaaattaaacatactatgtaggttgacaataactttaatccagtcaaaattcatatttctcctataagtctttaataaataatgataataaagaagacaaaaaatgtatttattgaccatatttcacatgtcagatgctgtgcaatgcattttacttcttttttatcttattaaatctcataatataaccatgacctggataccattattgtccctattatacaggtaaccttcaatcacataaaaattaggaacatttgaattcagagcttaaaacacaaacaacttgtacttttcaaaataacttgctgttacctttaaggtctgtcattaaaacatagcatgtcctgggctcagtacctaatctttcttatctgaaacttaaattcctatttattaacctttttagccctatattttctaattccatagaaaactaaaggttttggttagatcttcatgtggaatgactggtttcattcaatagacttaattcagcagtctgtggggaagagcaaggtatgatagaatggttcctcaagtgcttcagatgtgaagtgggtttaaatatactgtccctgtcttcttcagagttttggtaaagataaaataggacactcatttaaaagcaatctttgcaaatgacaagccactatagacattaatagagttttcatttccagtattatcattaatatcagatcctggaagaaggttgagccttgacctagagcaaaaaaacagaagaattagtaaaggaatcctggagaaagcccctgctgtgtatttaaaggagaaagggagatcatgttgggaaattataatattaaaagtaaacaaaagctaggaagtaaaataaaataaattatatggcctagatccccataagtaatggtttaacttctgccttcctgtgttctgagccagattagggcacagtagagaaagaggagtctctgaaaatgtttccaatttcgctggtcagacagcggatcatcagtgaatcagatgaaaatttgtggatttatgcactaactgatcagcaggaaattaaacaagaaaagcgttggtagctctggtgaatcccaaaagaatttggcagttgctagccatgctcctgaatatgtataaacagtacatcatatgactaagagtttgacttaggggttagattttatgtgtttgaaccccaaattagttatttaatagttggcaccccaaaacaagttacttaacctcactaagattcagttttcctgtttataaaatgtagatagtgatagtatgtactttataggattattgtgaaaaataaatgaaatatcagatttatttaggataacacctggcatatgtttggtattcagtaattagttgctgctgttttattctgctctcccttgcatcccacttttctaagttgtaaactaaatagttgtacacagattgacagattaagaaaggcttgtgattgtgctagacctatgcctctctctcaccagattccaggtgtatatgtggaggtgggatagggagtggagtaagtgggtaaatattaaattgcccagttgggcaccatcctgaatattatctctaaagaaagaagcaaaaccaggcacagctgatgggttaaccagatatgatacagaaaacatttccttctgctttttggttttaagcctatatttgaagccttagatctctccagcacagtaagcaccaggagtccatgaagaagatgg

SEQ ID NO: 6 - DNA, 50 bp, Human
tctcaatataataatattctttattcctggacagctcggttaatgaaaaa

SEQ ID NO: 7 - DNA, 2892 bp, Human
gaatcacgtgtccagaacactctgattcacagccaaggatccagaaggccaaggttttgttaaggggctactggaaaaatttctattctctccacagcctgctggttttacattagatttattcgcctgataagaatattttgtttctgctgcttctgtccaccttaatatgctccttctatttgtagatatgatagactcctatttttcttgttttatattatgaccacacacatctctgctggaaagtcaacatgtagtaagcaagatttaactgtttgattataactgtgcaaatacagaaaaaaagaaggctggctgaaagttgagttaaactttgacagtttgataatatttggttcttagggttttttttttttttagcattcttaatagttacagttgggcatgatttgtaccatccacccatacccacacagtcacagtcacacacacatatgtattacttacactatatataacttcctatgcaaatattttaccaccagtcaataatacatttttgccaagacatgaagttttataaagatctgtataattgcctgaatcaccagcacattcactgacatgatattatttgcagattgacaagtaggaagtggggaacttttattaagttactcgttgtctggggaggtaaataggttaaaaacagggaaattataagtgcagagattaacatttcacaaatgtttagtgaaacatttgtgaaaaaagaagactaaattaagacctgagctgaaataaagtgacgtggaaatggaaataatggttatatctaaaacatgtagaaaaagagtaactggtagattttgttaacaaattaaagaataaagttagacaagcaactggttgactaatacattaagcgtttgagtctaagatgaaaggagaacactggttatgttgatagaatgataaaaagggtcgggcgcggaggctcacgcctgtaatcccagccctttgggaggccgaggtgggcagatcacgaagtcagtagtttgagaccagcctggccaacatagtgaaaccccgtctctactaaaaatacaaaaaaaaaattagctgggtgtggtggcagtcacctgtagtcccagctacttgggaggatgaggcaggagaatcgcttgaacctgggaggcggaggttgcagtgagccgagatcgcaccagtgcactccagccttggtgacaatgggagactccatctcaaaaaaaaaaaaaaaaaaaaaaagataaaaagtcagaaatctgaaaagtggaggaagagtacaaatagacctaaattaagtctcattttttggctttgattttggggagacaaagggaaatgcagccatagagggcctgatgacatccaatacatgagttctggtaaagataaaatttgatacacggtttggtgtcattataagagaaatcattattaaatgaagcaagttaacactctaagagaattattttgagatagaagtgaagctaagctaaacttcacatgcctataattggagggaaaaactaaggataaaatctagcctagaagatacaataattagtcataaacatgcattgtgaaactgtagagagcaggtagcccaaaatagagaaagattagataaagagaaaataagtatccatcagagacagtatctctaggcttgggcaagagaaaagtccacagtgataagcaactccacctaaggcatgaatatgcggcagagaaaacagcaatagtgaatgaatgcaaaaggtgctgagcaaattccacacatgagtattgtgcatgagtaaatgaataaaacatttgcaaagacctttagagaaagagaatgggagcatatgtgcgaaataagatagttgattatgaatagaaggtagtgaagaaaagcaagctaagaaaaaattctgtttataaaagaaggaaaagatagtttatgtttttagcctaagtataagagtcctacagatggactgaaaaaaatcagtctgagagtattagtcacaattaatgaaataattacattttatgtattgaggatgccaagattaaaaggtgacaggtagatgttaatttccctagattgtgaaagtgatcacgacaatcacacaacaaataattaagtgacttggtatgctttatttaattgtagggcctgaggttttccattctcatttttctaaaatacaattttgtttctccaaatttgacagcagaataaaaaccctaccctttcactgtgtatcatgctaagctgcatctctactcttgatcatctgtaggtattaatcacatcacttccatggcatggatgttcacatacagactcttaaccctggtttaccaggacctctaggagtggatccaatctatatctttacagttgtatagtatatgatatctcttttatttcactcaatttatattttcatcattgactacatatttcttatacacaacacacaatttatgaattttttctcaagatcattctgagagttgccccaccctacctgccttttatagtacgcccacctcaggcagacacagagcacaatgctggggttctcttcacactatcactgccccaaattgtctttctaaatttcaacttcaatgtcatcttctccatgaagaccactgaatgaacaccttttcatccagccttaatttcttgctccataactactctatcccacgatgcagtattgtatcattaattattagtgtgcttgtgacctccttatgtattctcaattacctgtatttgtgcaataaattggaataatgtaacttgatttcttatctgtgtttgtgttggcatgcaagatttaggtacttatcaagataatggggaattaaggcatcaataaa

SEQ ID NO: 8 - DNA, 309 bp, Human
cctccacacagagcccatccgtcttccccttgacccgctgctgcaaaaacattccctccaatgccacctccgtgactctgggctgcctggccacgggctacttcccggagccggtgatggtgacctgggacacaggctccctcaacgggacaactatgaccttaccagccaccaccctcacgctctctggtcactatgccaccatcagcttgctgaccgtctcgggtgcgtgggccaagcagatgttcacctgccgtgtggcacacactccatcgtccacagactgggtcgacaacaaaaccttcagcg

SEQ ID NO: 9 - DNA, 321 bp, Human
tctgctccagggacttcaccccgcccaccgtgaagatcttacagtcgtcctgcgacggcggcgggcacttccccccgaccatccagctcctgtgcctcgtctctgggtacaccccagggactatcaacatcacctggctggaggacgggcaggtcatggacgtggacttgtccaccgcctctaccacgcaggagggtgagctggcctccacacaaagcgagctcaccctcagccagaagcactggctgtcagaccgcacctacacctgccaggtcacctatcaaggtcacacctttgaggacagcaccaagaagtgtgcag

SEQ ID NO: 10 - DNA, 324 bp, Human
attccaacccgagaggggtgagcgcctacctaagccggcccagcccgttcgacctgttcatccgcaagtcgcccacgatcacctgtctggtggtggacctggcacccagcaaggggaccgtgaacctgacctggtcccgggccagtgggaagcctgtgaaccactccaccagaaaggaggagaagcagcgcaatggcacgttaaccgtcacgtccaccctgccggtgggcacccgagactggatcgagggggagacctaccagtgcagggtgacccacccccacctgcccagggccctcatgcggtccacgaccaagaccagcg

SEQ ID NO: 11 - DNA, 332 bp, Human
gcccgcgtgctgccccggaagtctatgcgtttgcgacgccggagtggccggggagccgggacaagcgcaccctcgcctgcctgatccagaacttcatgcctgaggacatctcggtgcagtggctgcacaacgaggtgcagctcccggacgcccggcacagcacgacgcagccccgcaagaccaagggctccggcttcttcgtcttcagccgcctggaggtgaccagggccgaatgggagcagaaagatgagttcatctgccgtgcagtccatgaggcagcgagcccctcacagaccgtccagcgagcggtgtctgtaaatcccggtaaatga

SEQ ID NO: 12 - DNA, 21 bp, Human
aatccatctaagagctgaaat

SEQ ID NO: 13 - DNA, 21 bp, Human
aagagctgaaattgctgaaaa

SEQ ID NO: 14 - DNA, 21 bp, Human
aaaaaactactccctccaaaa

SEQ ID NO: 15 - DNA, 21 bp, Human
aaaactactccctccaaaagc

SEQ ID NO: 16 - DNA, 21 bp, Human
aactactccctccaaaagctg

SEQ ID NO: 17 - DNA, 21 bp, Human
aaaagctgtagagacaggcac

SEQ ID NO: 18 - DNA, 21 bp, Human
aagctgtagagacaggcacat

SEQ ID NO: 19 - DNA, 21 bp, Human
aagagaagctcctggagacac

SEQ ID NO: 20 - DNA, 21 bp, Human
aagctcctggagacacattgg

SEQ ID NO: 21 - DNA, 21 bp, Human
aacatttacctggtgattatg

SEQ ID NO: 22 - DNA, 21 bp, Human
aatgtctggaggacaaatgtg

SEQ ID NO: 23 - DNA, 21 bp, Human
aaatgtggactagggggaggg

SEQ ID NO: 24 - DNA, 21 bp, Human
aaaatagattccctatagcca

SEQ ID NO: 25 - DNA, 21 bp, Human
aatagattccctatagccatg

SEQ ID NO: 26 - DNA, 21 bp, Human
aactggggaggagtaatcacg

SEQ ID NO: 27 - DNA, 21 bp, Human
aatcacggagaaaagatgcac

SEQ ID NO: 28 - DNA, 21 bp, Human
aaagacttttctagaaagctc

SEQ ID NO: 29 - DNA, 21 bp, Human
aaagctcatccaagggaaggt

SEQ ID NO: 30 - DNA, 21 bp, Human
aagggaaggtattctccaaaa

SEQ ID NO: 31 - DNA, 21 bp, Human
aaaatcttagtttatgtgggg

SEQ ID NO: 32 - DNA, 21 bp, Human
aatcttagtttatgtggggga

SEQ ID NO: 33 - DNA, 21 bp, Human
aaattacagacccctcctcct

SEQ ID NO: 34 - DNA, 21 bp, Human
aaaattagccaagaggagtca

SEQ ID NO: 35 - DNA, 21 bp, Human
aattagccaagaggagtcaga

SEQ ID NO: 36 - DNA, 21 bp, Human
aagaggagtcagattcaaggc

SEQ ID NO: 37 - DNA, 21 bp, Human
aagggaggaaagagagaaaat

SEQ ID NO: 38 - DNA, 21 bp, Human
aaagagagaaaatcagctgta

SEQ ID NO: 39 - DNA, 21 bp, Human
aaaatcagctgtatcactgga

SEQ ID NO: 40 - DNA, 21 bp, Human
aatcagctgtatcactggaga

SEQ ID NO: 41 - DNA, 21 bp, Human
aaggtcactgctcaggagaag

SEQ ID NO: 42 - DNA, 21 bp, Human
aagagggcaaccaaaccaggg

SEQ ID NO: 43 - DNA, 21 bp, Human
aaccaaaccagggagagtcaa

SEQ ID NO: 44 - DNA, 21 bp, Human
aaaccagggagagtcaactgt

SEQ ID NO: 45 - DNA, 21 bp, Human
aactgtaagaacataccatgc

SEQ ID NO: 46 - DNA, 21 bp, Human
aagaacataccatgctcccct

SEQ ID NO: 47 - DNA, 21 bp, Human
aacataccatgctcccctgcc

SEQ ID NO: 48 - DNA, 21 bp, Human
aatatggattaaagagggcag

SEQ ID NO: 49 - DNA, 21 bp, Human
aaagagggcagtgtgattgct

SEQ ID NO: 50 - DNA, 21 bp, Human
aaagaaagtcacatactgagg

SEQ ID NO: 51 - DNA, 21 bp, Human
aaagtcacatactgaggccta

SEQ ID NO: 52 - DNA, 21 bp, Human
aagatactacgaagaaggaaa

SEQ ID NO: 53 - DNA, 21 bp, Human
aagaaggaaaaattaggggtc

SEQ ID NO: 54 - DNA, 21 bp, Human
aaggaaaaattaggggtccat

SEQ ID NO: 55 - DNA, 21 bp, Human
aaaaattaggggtccataact

SEQ ID NO: 56 - DNA, 21 bp, Human
aaattaggggtccataactgt

SEQ ID NO: 57 - DNA, 21 bp, Human
aactgtgaaaatcagccacag

SEQ ID NO: 58 - DNA, 21 bp, Human
aaaatcagccacagtgtgtgt

SEQ ID NO: 59 - DNA, 21 bp, Human
aatcagccacagtgtgtgtga

SEQ ID NO: 60 - DNA, 21 bp, Human
aacagcggacgtggagtgagc

SEQ ID NO: 61 - DNA, 21 bp, Human
aatccacatccatctgcagct

SEQ ID NO: 62 - DNA, 21 bp, Human
aagagccgaaatgagaaaaga

SEQ ID NO: 63 - DNA, 21 bp, Human
aaatgagaaaagagccacctc

SEQ ID NO: 64 - DNA, 21 bp, Human
aaaagagccacctccaacccc

SEQ ID NO: 65 - DNA, 21 bp, Human
aacccccccagagttttagcc

SEQ ID NO: 66 - DNA, 21 bp, Human
aagtggactccctggtgattt

SEQ ID NO: 67 - DNA, 21 bp, Human
aagaggacattgggtttccaa

SEQ ID NO: 68 - DNA, 21 bp, Human
aaaggcccattcactatttca

SEQ ID NO: 69 - DNA, 21 bp, Human
aagaagtggtgccatcagctc

SEQ ID NO: 70 - DNA, 21 bp, Human
aagtggtgccatcagctcatg

SEQ ID NO: 71 - DNA, 21 bp, Human
aaggagcattctgagccaggg

SEQ ID NO: 72 - DNA, 21 bp, Human
aaaaatgctctgtgagaccca

SEQ ID NO: 73 - DNA, 21 bp, Human
aaatgctctgtgagacccaat

SEQ ID NO: 74 - DNA, 21 bp, Human
aatgggaagctccctgcagtg

SEQ ID NO: 75 - DNA, 21 bp, Human
aagctccctgcagtgcaaggt

SEQ ID NO: 76 - DNA, 21 bp, Human
aacctaacaagtcaatgagaa

SEQ ID NO: 77 - DNA, 21 bp, Human
aacaagtcaatgagaatcaca

SEQ ID NO: 78 - DNA, 21 bp, Human
aagtcaatgagaatcacatgt

SEQ ID NO: 79 - DNA, 21 bp, Human
aataggtaggagatggagaaa

SEQ ID NO: 80 - DNA, 21 bp, Human
aattcagtgcttgccaatctt

SEQ ID NO: 81 - DNA, 21 bp, Human
aatcttaacagagacagtagt

SEQ ID NO: 82 - DNA, 21 bp, Human
aacagagacagtagtaagaca

SEQ ID NO: 83 - DNA, 21 bp, Human
aagacatgcagaaagcaaagc

SEQ ID NO: 84 - DNA, 21 bp, Human
aaagcaaagcccagaaaagta

SEQ ID NO: 85 - DNA, 21 bp, Human
aaagcccagaaaagtatgaag

SEQ ID NO: 86 - DNA, 21 bp, Human
aaaagtatgaaggtgtcaaag

SEQ ID NO: 87 - DNA, 21 bp, Human
aagtatgaaggtgtcaaagtg

SEQ ID NO: 88 - DNA, 21 bp, Human
aaggtgtcaaagtgccattta

SEQ ID NO: 89 - DNA, 21 bp, Human
aaagtgccatttaagtatggg

SEQ ID NO: 90 - DNA, 21 bp, Human
aagtatgggttcacttggagg

SEQ ID NO: 91 - DNA, 21 bp, Human
aacttgttttcagcagacaat

SEQ ID NO: 92 - DNA, 21 bp, Human
aaggggacacacatcattaaa

SEQ ID NO: 93 - DNA, 21 bp, Human
aaacaaggattgggacaggga

SEQ ID NO: 94 - DNA, 21 bp, Human
aaggattgggacagggacttc

SEQ ID NO: 95 - DNA, 21 bp, Human
aagtctagagctatgaaatag

SEQ ID NO: 96 - DNA, 21 bp, Human
aacctgagatttactgaagta

SEQ ID NO: 97 - DNA, 21 bp, Human
aagtaaatacagatctgtcct

SEQ ID NO: 98 - DNA, 21 bp, Human
aaatacagatctgtcctgtgc

SEQ ID NO: 99 - DNA, 21 bp, Human
aaccacatctgtcctctagag

SEQ ID NO: 100 - DNA, 21 bp, Human
aatcccctgagagctccgttc

SEQ ID NO: 101 - DNA, 21 bp, Human
aaagacttgcctctgaggaag

SEQ ID NO: 102 - DNA, 21 bp, Human
aagcatgctctctgctgtgct

SEQ ID NO: 103 - DNA, 21 bp, Human
aaggagctagaccaggaaaga

SEQ ID NO: 104 - DNA, 21 bp, Human
aatgtctcagagactggtcag

SEQ ID NO: 105 - DNA, 21 bp, Human
aacctgggttatcaacagagc

SEQ ID NO: 106 - DNA, 21 bp, Human
aacagagctctactgcactct

SEQ ID NO: 107 - DNA, 21 bp, Human
aataatccttgagggcacctg

SEQ ID NO: 108 - DNA, 21 bp, Human
aatccttgagggcacctgttc

SEQ ID NO: 109 - DNA, 21 bp, Human
aaacaacaacaggagcctcac

SEQ ID NO: 110 - DNA, 21 bp, Human
aaaacattctgaggggttcgg

SEQ ID NO: 111 - DNA, 21 bp, Human
aacatgtcttccccaagtcac

SEQ ID NO: 112 - DNA, 21 bp, Human
aaatctatcacgacctcctgc

SEQ ID NO: 113 - DNA, 21 bp, Human
aactgtcaacctggcaggcaa

SEQ ID NO: 114 - DNA, 21 bp, Human
aacctggcaggcaagatagga

SEQ ID NO: 115 - DNA, 21 bp, Human
aaggactcatcaactagccag

SEQ ID NO: 116 - DNA, 21 bp, Human
aactagccagtagcagtcact

SEQ ID NO: 117 - DNA, 21 bp, Human
aacatctagatgttgggcctg

SEQ ID NO: 118 - DNA, 21 bp, Human
aagtttatggctccagtggct

SEQ ID NO: 119 - DNA, 21 bp, Human
aaagggtgatggccacactac

SEQ ID NO: 120 - DNA, 21 bp, Human
aagtggaggtcagattgtggt

SEQ ID NO: 121 - DNA, 21 bp, Human
aacctatgtcccatgcatctg

SEQ ID NO: 122 - DNA, 21 bp, Human
aagtaggtgccacacaatgga

SEQ ID NO: 123 - DNA, 21 bp, Human
aatggaggctaactctagcac

SEQ ID NO: 124 - DNA, 21 bp, Human
aactctagcaccctctctctc

SEQ ID NO: 125 - DNA, 21 bp, Human
aacctgccttttcctgcaaac

SEQ ID NO: 126 - DNA, 21 bp, Human
aaagtgctggagcaccttgtt

SEQ ID NO: 127 - DNA, 21 bp, Human
aagatccctaccattcagacc

SEQ ID NO: 128 - DNA, 21 bp, Human
aagaatcctcccttaacccca

SEQ ID NO: 129 - DNA, 21 bp, Human
aatcctcccttaaccccacat

SEQ ID NO: 130 - DNA, 21 bp, Human
aaccccacatcttcctatacc

SEQ ID NO: 131 - DNA, 21 bp, Human
aaaacacttcccaaccggaac

SEQ ID NO: 132 - DNA, 21 bp, Human
aacacttcccaaccggaacac

SEQ ID NO: 133 - DNA, 21 bp, Human
aagcaggacctgtaagaggta

SEQ ID NO: 134 - DNA, 21 bp, Human
aagtggaagagagactagagc

SEQ ID NO: 135 - DNA, 21 bp, Human
aatctgcagagagtccccatt

SEQ ID NO: 136 - DNA, 21 bp, Human
aatgtcctcaccagatagacc

SEQ ID NO: 137 - DNA, 21 bp, Human
aacagtccccagagtgtgatg

SEQ ID NO: 138 - DNA, 21 bp, Human
aaccccatcaaaaagtgggcg

SEQ ID NO: 139 - DNA, 21 bp, Human
aaaaagtgggcgaaggacatg

SEQ ID NO: 140 - DNA, 21 bp, Human
aaagtgggcgaaggacatgaa

SEQ ID NO: 141 - DNA, 21 bp, Human
aaaatgctcaccatcactggc

SEQ ID NO: 142 - DNA, 21 bp, Human
aatgctcaccatcactggcca

SEQ ID NO: 143 - DNA, 21 bp, Human
aaacaacaggtgctggagagg

SEQ ID NO: 144 - DNA, 21 bp, Human
aacaggtgctggagaggatgt

SEQ ID NO: 145 - DNA, 21 bp, Human
aacccttgtggaagtcagtgt

SEQ ID NO: 146 - DNA, 21 bp, Human
aagtcagtgtggccattcctc

SEQ ID NO: 147 - DNA, 21 bp, Human
aaagtcttctccccaagggaa

SEQ ID NO: 148 - DNA, 21 bp, Human
aattaactcttgccccttggg

SEQ ID NO: 149 - DNA, 21 bp, Human
aactcttgccccttgggtcat

SEQ ID NO: 150 - DNA, 21 bp, Human
aaccctcattcataggtgcag

SEQ ID NO: 151 - DNA, 21 bp, Human
aatggccaccaaaatgtccca

SEQ ID NO: 152 - DNA, 21 bp, Human
aaggaagcccccttaaactgt

SEQ ID NO: 153 - DNA, 21 bp, Human
aagtttcagggcctcaaactg

SEQ ID NO: 154 - DNA, 21 bp, Human
aagatgtgcagaatcaggcca

SEQ ID NO: 155 - DNA, 21 bp, Human
aaggctgccttcagtattgca

SEQ ID NO: 156 - DNA, 21 bp, Human
aaacacacgggtcacagaact

SEQ ID NO: 157 - DNA, 21 bp, Human
aactgtttagccagagtcctg

SEQ ID NO: 158 - DNA, 21 bp, Human
aatgcaaatgtcctggtctgg

SEQ ID NO: 159 - DNA, 21 bp, Human
aaatgtcctggtctgggctaa

SEQ ID NO: 160 - DNA, 21 bp, Human
aatggggatttctctgtctcg

SEQ ID NO: 161 - DNA, 21 bp, Human
aacgccacttctgcagacatg

SEQ ID NO: 162 - DNA, 21 bp, Human
aaggttttgccttctggaccc

SEQ ID NO: 163 - DNA, 21 bp, Human
aaaggaggctctctctctgct

SEQ ID NO: 164 - DNA, 21 bp, Human
aaccacccttaaggtaggtac

SEQ ID NO: 165 - DNA, 21 bp, Human
aatcccagcttcttgggagga

SEQ ID NO: 166 - DNA, 21 bp, Human
aaggatcttcctcagtgggct

SEQ ID NO: 167 - DNA, 21 bp, Human
aagtttgaagccctttgggct

SEQ ID NO: 168 - DNA, 21 bp, Human
aaatgtggtccccaatgttgg

SEQ ID NO: 169 - DNA, 21 bp, Human
aagtgtttgggtcatggaggc

SEQ ID NO: 170 - DNA, 21 bp, Human
aacaggcagtcagcatctgct

SEQ ID NO: 171 - DNA, 21 bp, Human
aaactcctgttcagggtagca

SEQ ID NO: 172 - DNA, 21 bp, Human
aaatatcacgagcagacaggc

SEQ ID NO: 173 - DNA, 21 bp, Human
aacacactgtataggctgagc

SEQ ID NO: 174 - DNA, 21 bp, Human
aaaagttcctgcccaaagcct

SEQ ID NO: 175 - DNA, 21 bp, Human
aagttcctgcccaaagcctct

SEQ ID NO: 176 - DNA, 21 bp, Human
aacaggtccaagtttgggcat

SEQ ID NO: 177 - DNA, 21 bp, Human
aacgtgtgttcctgatgtgga

SEQ ID NO: 178 - DNA, 21 bp, Human
aaattagtagcagcccactcc

SEQ ID NO: 179 - DNA, 21 bp, Human
aacctgtggcattcaactgtg

SEQ ID NO: 180 - DNA, 21 bp, Human
aactgtgtatcagcccgttct

SEQ ID NO: 181 - DNA, 21 bp, Human
aaagtggcctccctaactcca

SEQ ID NO: 182 - DNA, 21 bp, Human
aatagtacctctcgatgagcc

SEQ ID NO: 183 - DNA, 21 bp, Human
aacgacttcagaggaagcctt

SEQ ID NO: 184 - DNA, 21 bp, Human
aatgactctctctgagggaca

SEQ ID NO: 185 - DNA, 21 bp, Human
aacccaaatttcccctggcaa

SEQ ID NO: 186 - DNA, 21 bp, Human
aatctttccccgtctcttcca

SEQ ID NO: 187 - DNA, 21 bp, Human
aaaacatagcatgtcctgggc

SEQ ID NO: 188 - DNA, 21 bp, Human
aacatagcatgtcctgggctc

SEQ ID NO: 189 - DNA, 21 bp, Human
aattcagcagtctgtggggaa

SEQ ID NO: 190 - DNA, 21 bp, Human
aagaaggttgagccttgacct

SEQ ID NO: 191 - DNA, 21 bp, Human
aaggttgagccttgacctaga

SEQ ID NO: 192 - DNA, 21 bp, Human
aaaggaatcctggagaaagcc

SEQ ID NO: 193 - DNA, 21 bp, Human
aacttctgccttcctgtgttc

SEQ ID NO: 194 - DNA, 21 bp, Human
aatttcgctggtcagacagcg

SEQ ID NO: 195 - DNA, 21 bp, Human
aaaagcgttggtagctctggt

SEQ ID NO: 196 - DNA, 21 bp, Human
aagcgttggtagctctggtga

SEQ ID NO: 197 - DNA, 21 bp, Human
aaattgcccagttgggcacca

SEQ ID NO: 198 - DNA, 21 bp, Human
aagcaaaaccaggcacagctg

SEQ ID NO: 199 - DNA, 21 bp, Human
aaaaccaggcacagctgatgg

SEQ ID NO: 200 - DNA, 21 bp, Human
aagccttagatctctccagca

SEQ ID NO: 201 - DNA, 21 bp, Human
aacattctgaggggttcgggc

SEQ ID NO: 202 - DNA, 21 bp, Human
aagccccccttgccatgtgaa

SEQ ID NO: 203 - DNA, 21 bp, Human
aagccctttgggcttcttggc

SEQ ID NO: 204 - DNA, 21 bp, Human
aatgttggaggtggggcctag

SEQ ID NO: 205 - DNA, 21 bp, Human
aaatggcttggtgccttcccc

SEQ ID NO: 206 - DNA, 21 bp, Human
aactgtgtgccctgggacaag

SEQ ID NO: 207 - DNA, 21 bp, Human
aaagcctctcacgctggatcc

SEQ ID NO: 208 - DNA, 21 bp, Human
aatcctggagaaagcccctgc

SEQ ID NO: 209 - DNA, 21 bp, Human
aaccaggcacagctgatgggt

SEQ ID NO: 210 - DNA, 21 bp, Human
aaatctaagcatgctctctgc

SEQ ID NO: 211 - DNA, 21 bp, Human
aatgaggatgtgataaggagc

SEQ ID NO: 212 - DNA, 21 bp, Human
aaatgactggtctggtacact

SEQ ID NO: 213 - DNA, 21 bp, Human
aaatgctcttctgccttggtt

SEQ ID NO: 214 - DNA, 21 bp, Human
aactaaagcagcagcagaaac

SEQ ID NO: 215 - DNA, 21 bp, Human
aaagcagcagcagaaacaaca

SEQ ID NO: 216 - DNA, 21 bp, Human
aacaacaggagcctcacaatt

SEQ ID NO: 217 - DNA, 21 bp, Human
aacaggagcctcacaatttag

SEQ ID NO: 218 - DNA, 21 bp, Human
aaggcaaggttactgtagatc

SEQ ID NO: 219 - DNA, 21 bp, Human
aagataggagagtgaaggact

SEQ ID NO: 220 - DNA, 21 bp, Human
aagccaccttaggaccaaatt

SEQ ID NO: 221 - DNA, 21 bp, Human
aaaacatctagatgttgggcc

SEQ ID NO: 222 - DNA, 21 bp, Human
aagtctcaacttgtgaggtgt

SEQ ID NO: 223 - DNA, 21 bp, Human
aagaaagcaactctccatgct

SEQ ID NO: 224 - DNA, 21 bp, Human
aaagcaactctccatgctgta

SEQ ID NO: 225 - DNA, 21 bp, Human
aactctccatgctgtattagc

SEQ ID NO: 226 - DNA, 21 bp, Human
aactgctattcctgtgtgaac

SEQ ID NO: 227 - DNA, 21 bp, Human
aacactacctcataagtggag

SEQ ID NO: 228 - DNA, 21 bp, Human
aaatataggtgctctcatggg

SEQ ID NO: 229 - DNA, 21 bp, Human
aagaacctatgtcccatgcat

SEQ ID NO: 230 - DNA, 21 bp, Human
aactcaggggtacttaagtag

SEQ ID NO: 231 - DNA, 21 bp, Human
aaaccctagcagtgtcagatt

SEQ ID NO: 232 - DNA, 21 bp, Human
aaatgttgtggccaaagtgct

SEQ ID NO: 233 - DNA, 21 bp, Human
aagcttcaaatcctggctttg

SEQ ID NO: 234 - DNA, 21 bp, Human
aaatcctggctttgccattca

SEQ ID NO: 235 - DNA, 21 bp, Human
aatctaaacctccctacctca

SEQ ID NO: 236 - DNA, 21 bp, Human
aaggatgctgatagtacctgt

SEQ ID NO: 237 - DNA, 21 bp, Human
aatgcctgaatacagtcagtg

SEQ ID NO: 238 - DNA, 21 bp, Human
aactaccaaggaacagctaac

SEQ ID NO: 239 - DNA, 21 bp, Human
aatgagtgaaagccacttctc

SEQ ID NO: 240 - DNA, 21 bp, Human
aaagccacttctctcttcttg

SEQ ID NO: 241 - DNA, 21 bp, Human
aacaagaatcctcccttaacc

SEQ ID NO: 242 - DNA, 21 bp, Human
aatattgtccgcacatgttgc

SEQ ID NO: 243 - DNA, 21 bp, Human
aactgcctatttagcacagac

SEQ ID NO: 244 - DNA, 21 bp, Human
aaccggaacacttggttgatt

SEQ ID NO: 245 - DNA, 21 bp, Human
aacacttggttgatttctccc

SEQ ID NO: 246 - DNA, 21 bp, Human
aatgggttatcatgggagtga

SEQ ID NO: 247 - DNA, 21 bp, Human
aagaagtggaagagagactag

SEQ ID NO: 248 - DNA, 21 bp, Human
aagagagactagagcttacag

SEQ ID NO: 249 - DNA, 21 bp, Human
aagaaagaatgtcctcaccag

SEQ ID NO: 250 - DNA, 21 bp, Human
aacttcacagtctccagaact

SEQ ID NO: 251 - DNA, 21 bp, Human
aatccagtctatcactgttgg

SEQ ID NO: 252 - DNA, 21 bp, Human
aacctactcatctgacaaagg

SEQ ID NO: 253 - DNA, 21 bp, Human
aaggacatgaacagacacttc

SEQ ID NO: 254 - DNA, 21 bp, Human
aatgagataccatctcacacc

SEQ ID NO: 255 - DNA, 21 bp, Human
aaagtcaggaaacaacaggtg

SEQ ID NO: 256 - DNA, 21 bp, Human
aaactagttcaacccttgtgg

SEQ ID NO: 257 - DNA, 21 bp, Human
aaataccatttgacccagcca

SEQ ID NO: 258 - DNA, 21 bp, Human
aaagacacatgcacacgtatg

SEQ ID NO: 259 - DNA, 21 bp, Human
aacagattgtccttgtacgtc

SEQ ID NO: 260 - DNA, 21 bp, Human
aaacgttagtcacaagctgtc

SEQ ID NO: 261 - DNA, 21 bp, Human
aagggaaaagatcttgtctgc

SEQ ID NO: 262 - DNA, 21 bp, Human
aattcaagctcagagattgcc

SEQ ID NO: 263 - DNA, 21 bp, Human
aagctcagagattgcctgttt

SEQ ID NO: 264 - DNA, 21 bp, Human
aacgtgactgtgctattacct

SEQ ID NO: 265 - DNA, 21 bp, Human
aaaaccctcattcataggtgc

SEQ ID NO: 266 - DNA, 21 bp, Human
aatttgtcggtcactagatgc

SEQ ID NO: 267 - DNA, 21 bp, Human
aatctgcaagtgtttcccgtt

SEQ ID NO: 268 - DNA, 21 bp, Human
aagtgtttcccgttttggaag

SEQ ID NO: 269 - DNA, 21 bp, Human
aatatcactggaaactgcgga

SEQ ID NO: 270 - DNA, 21 bp, Human
aaggcataaacctgtaaccac

SEQ ID NO: 271 - DNA, 21 bp, Human
aagtggtgctcaatgatgcta

SEQ ID NO: 272 - DNA, 21 bp, Human
aaactggatctacctctgcta

SEQ ID NO: 273 - DNA, 21 bp, Human
aacagtgtagctacaggtgat

SEQ ID NO: 274 - DNA, 21 bp, Human
aaaagatgtgcagaatcaggc

SEQ ID NO: 275 - DNA, 21 bp, Human
aaaggccttttgtaggtggta

SEQ ID NO: 276 - DNA, 21 bp, Human
aaagtttcagaatgcgtcctg

SEQ ID NO: 277 - DNA, 21 bp, Human
aagatctctcatccttgtctg

SEQ ID NO: 278 - DNA, 21 bp, Human
aacggcctaaaaggattcctt

SEQ ID NO: 279 - DNA, 21 bp, Human
aataaacctggcctacactac

SEQ ID NO: 280 - DNA, 21 bp, Human
aaacctggcctacactacatt

SEQ ID NO: 281 - DNA, 21 bp, Human
aaacttcctaacgccacttct

SEQ ID NO: 282 - DNA, 21 bp, Human
aacaaggttttgccttctgga

SEQ ID NO: 283 - DNA, 21 bp, Human
aaccacctcaccacattaact

SEQ ID NO: 284 - DNA, 21 bp, Human
aaggctgaggcaaaaagagtt

SEQ ID NO: 285 - DNA, 21 bp, Human
aaagagtttgagtggtcacag

SEQ ID NO: 286 - DNA, 21 bp, Human
aagacaccacaaagatgcttc

SEQ ID NO: 287 - DNA, 21 bp, Human
aaagatgcttcatcggcaaag

SEQ ID NO: 288 - DNA, 21 bp, Human
aaagtgtgagtggctgattac

SEQ ID NO: 289 - DNA, 21 bp, Human
aagacaggcagcttgatatct

SEQ ID NO: 290 - DNA, 21 bp, Human
aatgagttcacatgagagctg

SEQ ID NO: 291 - DNA, 21 bp, Human
aaaggaaactagaacctccct

SEQ ID NO: 292 - DNA, 21 bp, Human
aacctcccttttcactctctt

SEQ ID NO: 293 - DNA, 21 bp, Human
aaaatctaacaggcagtcagc

SEQ ID NO: 294 - DNA, 21 bp, Human
aatctaacaggcagtcagcat

SEQ ID NO: 295 - DNA, 21 bp, Human
aaatgcagctgatatcctcct

SEQ ID NO: 296 - DNA, 21 bp, Human
aaaggaaagaatccctctctc

SEQ ID NO: 297 - DNA, 21 bp, Human
aaagaatccctctctcctttc

SEQ ID NO: 298 - DNA, 21 bp, Human
aatccctctctcctttcttct

SEQ ID NO: 299 - DNA, 21 bp, Human
aacagtaccaacattgtgtgg

SEQ ID NO: 300 - DNA, 21 bp, Human
aacattgtgtggctgtgagtt

SEQ ID NO: 301 - DNA, 21 bp, Human
aaggaatttctgcatgaggac

SEQ ID NO: 302 - DNA, 21 bp, Human
aaggaagccagcagattgatt

SEQ ID NO: 303 - DNA, 21 bp, Human
aagccagcagattgatttggt

SEQ ID NO: 304 - DNA, 21 bp, Human
aaacacaaaagttcctgccca

SEQ ID NO: 305 - DNA, 21 bp, Human
aactgaaatttagacacgccc

SEQ ID NO: 306 - DNA, 21 bp, Human
aaaacctgtggcattcaactg

SEQ ID NO: 307 - DNA, 21 bp, Human
aagatgggtagtttggttgct

SEQ ID NO: 308 - DNA, 21 bp, Human
aataccgggcacttaagcaaa

SEQ ID NO: 309 - DNA, 21 bp, Human
aaatgaaagtggcctccctaa

SEQ ID NO: 310 - DNA, 21 bp, Human
aactaggaatagtacctctcg

SEQ ID NO: 311 - DNA, 21 bp, Human
aaaatgactctctctgaggga

SEQ ID NO: 312 - DNA, 21 bp, Human
aaaggaacccaaatttcccct

SEQ ID NO: 313 - DNA, 21 bp, Human
aaatttcccctggcaaagtct

SEQ ID NO: 314 - DNA, 21 bp, Human
aaagtagaatctttccccgtc

SEQ ID NO: 315 - DNA, 21 bp, Human
aaatattgccctctgacagca

SEQ ID NO: 316 - DNA, 21 bp, Human
aaccatgacctggataccatt

SEQ ID NO: 317 - DNA, 21 bp, Human
aatggttcctcaagtgcttca

SEQ ID NO: 318 - DNA, 21 bp, Human
aagtgcttcagatgtgaagtg

SEQ ID NO: 319 - DNA, 21 bp, Human
aaagcccctgctgtgtattta

SEQ ID NO: 320 - DNA, 21 bp, Human
aaaggagaaagggagatcatg

SEQ ID NO: 321 - DNA, 21 bp, Human
aaagggagatcatgttgggaa

SEQ ID NO: 322 - DNA, 21 bp, Human
aagaaaagcgttggtagctct

SEQ ID NO: 323 - DNA, 21 bp, Human
aagaatttggcagttgctagc

SEQ ID NO: 324 - DNA, 21 bp, Human
aatttggcagttgctagccat

SEQ ID NO: 325 - DNA, 21 bp, Human
aatagttggcaccccaaaaca

SEQ ID NO: 326 - DNA, 21 bp, Human
aacacctggcatatgtttggt

SEQ ID NO: 327 - DNA, 21 bp, Human
aagaaaggcttgtgattgtgc

SEQ ID NO: 328 - DNA, 21 bp, Human
aaaggcttgtgattgtgctag

SEQ ID NO: 329 - DNA, 21 bp, Human
aaagaagcaaaaccaggcaca

SEQ ID NO: 330 - DNA, 21 bp, Human
aattgctcatttgcctctcta

SEQ ID NO: 331 - DNA, 21 bp, Human
aagaatggagtaatatgggag

SEQ ID NO: 332 - DNA, 21 bp, Human
aatggagtaatatgggagcaa

SEQ ID NO: 333 - DNA, 21 bp, Human
aagtaatcttctggcatgcat

SEQ ID NO: 334 - DNA, 21 bp, Human
aacactattaatcactctccc

SEQ ID NO: 335 - DNA, 21 bp, Human
aatcactctccctcttgaaat

SEQ ID NO: 336 - DNA, 21 bp, Human
aagcgatggtgaatgtcattt

SEQ ID NO: 337 - DNA, 21 bp, Human
aaccatccactaactctatct

SEQ ID NO: 338 - DNA, 21 bp, Human
aattctctctgtgttactacc

SEQ ID NO: 339 - DNA, 21 bp, Human
aattgactttctccccatgat

SEQ ID NO: 340 - DNA, 21 bp, Human
aaataacatgtcttccccaag

SEQ ID NO: 341 - DNA, 21 bp, Human
aaatatcccatggttatcgtc

SEQ ID NO: 342 - DNA, 21 bp, Human
aactgaactgttatcttcctg

SEQ ID NO: 343 - DNA, 21 bp, Human
aactgttatcttcctgaactc

SEQ ID NO: 344 - DNA, 21 bp, Human
aactcctgcaattctaattgc

SEQ ID NO: 345 - DNA, 21 bp, Human
aactgtgttgaaagaagtctc

SEQ ID NO: 346 - DNA, 21 bp, Human
aaagaagtctcaacttgtgag

SEQ ID NO: 347 - DNA, 21 bp, Human
aacttgtgaggtgtaaacatg

SEQ ID NO: 348 - DNA, 21 bp, Human
aaattactactactgctgtgg

SEQ ID NO: 349 - DNA, 21 bp, Human
aattcttttcagtctccagca

SEQ ID NO: 350 - DNA, 21 bp, Human
aagaagaaagcaactctccat

SEQ ID NO: 351 - DNA, 21 bp, Human
aacctttttgctagttgtcac

SEQ ID NO: 352 - DNA, 21 bp, Human
aatacctttgtaggttctcca

SEQ ID NO: 353 - DNA, 21 bp, Human
aattctactttcctctgctct

SEQ ID NO: 354 - DNA, 21 bp, Human
aaaagatgaccacaaactctc

SEQ ID NO: 355 - DNA, 21 bp, Human
aagatgaccacaaactctcaa

SEQ ID NO: 356 - DNA, 21 bp, Human
aaactctcaacttattggagg

SEQ ID NO: 357 - DNA, 21 bp, Human
aagttatcccaaatgttgtgg

SEQ ID NO: 358 - DNA, 21 bp, Human
aatgcctttcttcctcatgtt

SEQ ID NO: 359 - DNA, 21 bp, Human
aaacctccctacctcattaaa

SEQ ID NO: 360 - DNA, 21 bp, Human
aaacaaggatgctgatagtac

SEQ ID NO: 361 - DNA, 21 bp, Human
aaagcacaatgcctgaataca

SEQ ID NO: 362 - DNA, 21 bp, Human
aacacatcctagtttctccat

SEQ ID NO: 363 - DNA, 21 bp, Human
aaagtcattgtggtttggatg

SEQ ID NO: 364 - DNA, 21 bp, Human
aactcccttctgcatttttgt

SEQ ID NO: 365 - DNA, 21 bp, Human
aaggaacagctaacacaaatc

SEQ ID NO: 366 - DNA, 21 bp, Human
aaggttttctcttggcttagt

SEQ ID NO: 367 - DNA, 21 bp, Human
aacttctcagaatattgtccg

SEQ ID NO: 368 - DNA, 21 bp, Human
aaatttcctagagttctgctc

SEQ ID NO: 369 - DNA, 21 bp, Human
aaagtatgccagtgattctct

SEQ ID NO: 370 - DNA, 21 bp, Human
aaactaaaacacttcccaacc

SEQ ID NO: 371 - DNA, 21 bp, Human
aaatatagtacttccccagtc

SEQ ID NO: 372 - DNA, 21 bp, Human
aatctgtcagtaagtcccatt

SEQ ID NO: 373 - DNA, 21 bp, Human
aagcatttcccaatatctgct

SEQ ID NO: 374 - DNA, 21 bp, Human
aacctacagattacatgacga

SEQ ID NO: 375 - DNA, 21 bp, Human
aataatgggttatcatgggag

SEQ ID NO: 376 - DNA, 21 bp, Human
aaaagaagtggaagagagact

SEQ ID NO: 377 - DNA, 21 bp, Human
aatctcggtgccacctcggga

SEQ ID NO: 378 - DNA, 21 bp, Human
aaagaatgtcctcaccagata

SEQ ID NO: 379 - DNA, 21 bp, Human
aagacatacatgaagagccat

SEQ ID NO: 380 - DNA, 21 bp, Human
aaactgtattaagcaggggaa

SEQ ID NO: 381 - DNA, 21 bp, Human
aatctcgttttatgccattgg

SEQ ID NO: 382 - DNA, 21 bp, Human
aagttttagggtacatatgcc

SEQ ID NO: 383 - DNA, 21 bp, Human
aactcgtcatttagcattagg

SEQ ID NO: 384 - DNA, 21 bp, Human
aaagctatccctcccccctcc

SEQ ID NO: 385 - DNA, 21 bp, Human
aattcccatctatgagtgaga

SEQ ID NO: 386 - DNA, 21 bp, Human
aatttcatccatgtccctaca

SEQ ID NO: 387 - DNA, 21 bp, Human
aaaggacatgaactcatcact

SEQ ID NO: 388 - DNA, 21 bp, Human
aaagggctaatatccagaatc

SEQ ID NO: 389 - DNA, 21 bp, Human
aaacaaccccatcaaaaagtg

SEQ ID NO: 390 - DNA, 21 bp, Human
aagaagacatttatgcagcca

SEQ ID NO: 391 - DNA, 21 bp, Human
aaaaaatgctcaccatcactg

SEQ ID NO: 392 - DNA, 21 bp, Human
aaccacaatgagataccatct

SEQ ID NO: 393 - DNA, 21 bp, Human
aaaaagtcaggaaacaacagg

SEQ ID NO: 394 - DNA, 21 bp, Human
aatagcaaagacttggaacct

SEQ ID NO: 395 - DNA, 21 bp, Human
aaagacttggaaccttctctt

SEQ ID NO: 396 - DNA, 21 bp, Human
aacttcccactgcttgaaaat

SEQ ID NO: 397 - DNA, 21 bp, Human
aattcttgttccagcctttgt

SEQ ID NO: 398 - DNA, 21 bp, Human
aaatggcctttcttaacccta

SEQ ID NO: 399 - DNA, 21 bp, Human
aaaacagattgtccttgtacg

SEQ ID NO: 400 - DNA, 21 bp, Human
aagctgtcactgttctgttta

SEQ ID NO: 401 - DNA, 21 bp, Human
aatagaaagtcttctccccaa

SEQ ID NO: 402 - DNA, 21 bp, Human
aaatgacagagtgcttggaat

SEQ ID NO: 403 - DNA, 21 bp, Human
aaagatcaagtaacaggtctg

SEQ ID NO: 404 - DNA, 21 bp, Human
aagtaacaggtctgtatccat

SEQ ID NO: 405 - DNA, 21 bp, Human
aaagagggttgttctgtgttt

SEQ ID NO: 406 - DNA, 21 bp, Human
aaatggtcatctgaatgatcc

SEQ ID NO: 407 - DNA, 21 bp, Human
aaacactattctacgggaatg

SEQ ID NO: 408 - DNA, 21 bp, Human
aagagacaaaggaaaaagctg

SEQ ID NO: 409 - DNA, 21 bp, Human
aaactgcggatcagggcccct

SEQ ID NO: 410 - DNA, 21 bp, Human
aaacctgtaaccacactacaa

SEQ ID NO: 411 - DNA, 21 bp, Human
aaccacactacaaatagtgag

SEQ ID NO: 412 - DNA, 21 bp, Human
aaactgcatactctatgcact

SEQ ID NO: 413 - DNA, 21 bp, Human
aataagtggtgctcaatgatg

SEQ ID NO: 414 - DNA, 21 bp, Human
aatatctctgtctgaggaaga

SEQ ID NO: 415 - DNA, 21 bp, Human
aaaaggaagaggatagtggaa

SEQ ID NO: 416 - DNA, 21 bp, Human
aaggaagaggatagtggaata

SEQ ID NO: 417 - DNA, 21 bp, Human
aagaggatagtggaatatctg

SEQ ID NO: 418 - DNA, 21 bp, Human
aagcccccttaaactgtttaa

SEQ ID NO: 419 - DNA, 21 bp, Human
aaactgtttaagtttcagggc

SEQ ID NO: 420 - DNA, 21 bp, Human
aagccaaggataatctttcct

SEQ ID NO: 421 - DNA, 21 bp, Human
aaaatatctggctgcacattg

SEQ ID NO: 422 - DNA, 21 bp, Human
aatatctggctgcacattgaa

SEQ ID NO: 423 - DNA, 21 bp, Human
aatgctacaaatgtctcctgt

SEQ ID NO: 424 - DNA, 21 bp, Human
aaacaagcaaccctcaataca

SEQ ID NO: 425 - DNA, 21 bp, Human
aagcaaccctcaatacacata

SEQ ID NO: 426 - DNA, 21 bp, Human
aaaatgcaaatgtcctggtct

SEQ ID NO: 427 - DNA, 21 bp, Human
aagcataaaataaacctggcc

SEQ ID NO: 428 - DNA, 21 bp, Human
aaaataaacctggcctacact

SEQ ID NO: 429 - DNA, 21 bp, Human
aagtctaaccaaaggcttctt

SEQ ID NO: 430 - DNA, 21 bp, Human
aaccaaaggcttcttgcaaaa

SEQ ID NO: 431 - DNA, 21 bp, Human
aaaaagagtttgagtggtcac

SEQ ID NO: 432 - DNA, 21 bp, Human
aactttccttaaggatcttcc

SEQ ID NO: 433 - DNA, 21 bp, Human
aatgtcagctcacttaacatc

SEQ ID NO: 434 - DNA, 21 bp, Human
aacatctgtatctgttggaag

SEQ ID NO: 435 - DNA, 21 bp, Human
aaggtaagtttgaagcccttt

SEQ ID NO: 436 - DNA, 21 bp, Human
aaggtagaggtcatgatcaat

SEQ ID NO: 437 - DNA, 21 bp, Human
aactacaagacaccacaaaga

SEQ ID NO: 438 - DNA, 21 bp, Human
aagagtgtagattatgctcag

SEQ ID NO: 439 - DNA, 21 bp, Human
aaactagaacctcccttttca

SEQ ID NO: 440 - DNA, 21 bp, Human
aaagtcactgactttcttgtg

SEQ ID NO: 441 - DNA, 21 bp, Human
aaaaaggaaagaatccctctc

SEQ ID NO: 442 - DNA, 21 bp, Human
aacataccaacagtaccaaca

SEQ ID NO: 443 - DNA, 21 bp, Human
aacccttgaatccaaggaatt

SEQ ID NO: 444 - DNA, 21 bp, Human
aatccaaggaatttctgcatg

SEQ ID NO: 445 - DNA, 21 bp, Human
aatttctgcatgaggactatg

SEQ ID NO: 446 - DNA, 21 bp, Human
aatccatgtgcacttaatcac

SEQ ID NO: 447 - DNA, 21 bp, Human
aagacatattgccaggttcaa

SEQ ID NO: 448 - DNA, 21 bp, Human
aagttacttaacctctcatgc

SEQ ID NO: 449 - DNA, 21 bp, Human
aacctctcatgctttgatttc

SEQ ID NO: 450 - DNA, 21 bp, Human
aactttgagagtgagaaggaa

SEQ ID NO: 451 - DNA, 21 bp, Human
aaggacataaaggctaacaca

SEQ ID NO: 452 - DNA, 21 bp, Human
aaaggctaacacactgtatag

SEQ ID NO: 453 - DNA, 21 bp, Human
aaggctcccaacatcaaataa

SEQ ID NO: 454 - DNA, 21 bp, Human
aaataacaggtccaagtttgg

SEQ ID NO: 455 - DNA, 21 bp, Human
aagtttgggcatagtgatact

SEQ ID NO: 456 - DNA, 21 bp, Human
aaatttagacacgccctattg

SEQ ID NO: 457 - DNA, 21 bp, Human
aaaaaacctgtggcattcaac

SEQ ID NO: 458 - DNA, 21 bp, Human
aatgtgttccttctgtgttct

SEQ ID NO: 459 - DNA, 21 bp, Human
aatggtttgcttttttggggt

SEQ ID NO: 460 - DNA, 21 bp, Human
aatgagctgcttttattggac

SEQ ID NO: 461 - DNA, 21 bp, Human
aaaaaggaacccaaatttccc

SEQ ID NO: 462 - DNA, 21 bp, Human
aacttgctgttacctttaagg

SEQ ID NO: 463 - DNA, 21 bp, Human
aaggtatgatagaatggttcc

SEQ ID NO: 464 - DNA, 21 bp, Human
aaatatactgtccctgtcttc

SEQ ID NO: 465 - DNA, 21 bp, Human
aatctttgcaaatgacaagcc

SEQ ID NO: 466 - DNA, 21 bp, Human
aaatgacaagccactatagac

SEQ ID NO: 467 - DNA, 21 bp, Human
aatatcagatcctggaagaag

SEQ ID NO: 468 - DNA, 21 bp, Human
aaattatatggcctagatccc

SEQ ID NO: 469 - DNA, 21 bp, Human
aatggtttaacttctgccttc

SEQ ID NO: 470 - DNA, 21 bp, Human
aaagaggagtctctgaaaatg

SEQ ID NO: 471 - DNA, 21 bp, Human
aatgtttccaatttcgctggt

SEQ ID NO: 472 - DNA, 21 bp, Human
aaacaagaaaagcgttggtag

SEQ ID NO: 473 - DNA, 21 bp, Human
aatcccaaaagaatttggcag

SEQ ID NO: 474 - DNA, 21 bp, Human
aagagtttgacttaggggtta

SEQ ID NO: 475 - DNA, 21 bp, Human
aaagaaagaagcaaaaccagg

SEQ ID NO: 476 - DNA, 21 bp, Human
aattaattgctcatttgcctc

SEQ ID NO: 477 - DNA, 21 bp, Human
aaaaatctaagcatgctctct

SEQ ID NO: 478 - DNA, 21 bp, Human
aaagagcagtgaactaaacta

SEQ ID NO: 479 - DNA, 21 bp, Human
aataagtaatcttctggcatg

SEQ ID NO: 480 - DNA, 21 bp, Human
aatcttctggcatgcatttta

SEQ ID NO: 481 - DNA, 21 bp, Human
aattgcttacattcaccagaa

SEQ ID NO: 482 - DNA, 21 bp, Human
aaagccatatagaaatgactg

SEQ ID NO: 483 - DNA, 21 bp, Human
aagtttgagtaatgtctcaga

SEQ ID NO: 484 - DNA, 21 bp, Human
aattaccaaattcatcggact

SEQ ID NO: 485 - DNA, 21 bp, Human
aaattcatcggactattcttg

SEQ ID NO: 486 - DNA, 21 bp, Human
aattcagttaacctgggttat

SEQ ID NO: 487 - DNA, 21 bp, Human
aacaaatatcccatggttatc

SEQ ID NO: 488 - DNA, 21 bp, Human
aactcctttctttctttgact

SEQ ID NO: 489 - DNA, 21 bp, Human
aaggttactgtagatcttgaa

SEQ ID NO: 490 - DNA, 21 bp, Human
aattgccagaaaacatctaga

SEQ ID NO: 491 - DNA, 21 bp, Human
aaaacagtctcattggagtta

SEQ ID NO: 492 - DNA, 21 bp, Human
aacagtctcattggagttatt

SEQ ID NO: 493 - DNA, 21 bp, Human
aaaattcttttcagtctccag

SEQ ID NO: 494 - DNA, 21 bp, Human
aatcaattctactttcctctg

SEQ ID NO: 495 - DNA, 21 bp, Human
aatatggcttaagttcttgtc

SEQ ID NO: 496 - DNA, 21 bp, Human
aagttcttgtcactttccttt

SEQ ID NO: 497 - DNA, 21 bp, Human
aagtcaaaagatgaccacaaa

SEQ ID NO: 498 - DNA, 21 bp, Human
aaaaacaaggatgctgatagt

SEQ ID NO: 499 - DNA, 21 bp, Human
aaataggataaagcacaatgc

SEQ ID NO: 500 - DNA, 21 bp, Human
aatattaacagttacctgtgc

SEQ ID NO: 501 - DNA, 21 bp, Human
aacagttacctgtgcttatat

SEQ ID NO: 502 - DNA, 21 bp, Human
aataaagtcattgtggtttgg

SEQ ID NO: 503 - DNA, 21 bp, Human
aaatacattgcagtttctctg

SEQ ID NO: 504 - DNA, 21 bp, Human
aagccaaggaataatgtcaat

SEQ ID NO: 505 - DNA, 21 bp, Human
aacattgaagttggttgaaac

SEQ ID NO: 506 - DNA, 21 bp, Human
aacagctaacacaaatcttac

SEQ ID NO: 507 - DNA, 21 bp, Human
aacacaaatcttactagcaca

SEQ ID NO: 508 - DNA, 21 bp, Human
aaatcttactagcacagtaga

SEQ ID NO: 509 - DNA, 21 bp, Human
aacatggcaaggaatagaata

SEQ ID NO: 510 - DNA, 21 bp, Human
aaggaatagaatattctgcac

SEQ ID NO: 511 - DNA, 21 bp, Human
aagtgtatcttgagcatgttt

SEQ ID NO: 512 - DNA, 21 bp, Human
aacctttgaaagtgatcaagt

SEQ ID NO: 513 - DNA, 21 bp, Human
aaagtgatcaagtcctagatt

SEQ ID NO: 514 - DNA, 21 bp, Human
aactgcatagttcatttggta

SEQ ID NO: 515 - DNA, 21 bp, Human
aatgtttatgttactgctctg

SEQ ID NO: 516 - DNA, 21 bp, Human
aacagtctttttttcctctca

SEQ ID NO: 517 - DNA, 21 bp, Human
aaacacctgtgttatgttcaa

SEQ ID NO: 518 - DNA, 21 bp, Human
aaattgaatgagtgaaagcca

SEQ ID NO: 519 - DNA, 21 bp, Human
aagggcttaacttcttcaatt

SEQ ID NO: 520 - DNA, 21 bp, Human
aacttatacttcccttattcc

SEQ ID NO: 521 - DNA, 21 bp, Human
aacaaatacaatccagcatct

SEQ ID NO: 522 - DNA, 21 bp, Human
aaaacaagaatcctcccttaa

SEQ ID NO: 523 - DNA, 21 bp, Human
aaagtcccaaaaacttccata

SEQ ID NO: 524 - DNA, 21 bp, Human
aacgatttcctctagaattgt

SEQ ID NO: 525 - DNA, 21 bp, Human
aattgtactctcttcttggtt

SEQ ID NO: 526 - DNA, 21 bp, Human
aattccctgtgagttttctta

SEQ ID NO: 527 - DNA, 21 bp, Human
aattactgtctacccagttat

SEQ ID NO: 528 - DNA, 21 bp, Human
aaaacctacagattacatgac

SEQ ID NO: 529 - DNA, 21 bp, Human
aatgtgggtgtatacacatat

SEQ ID NO: 530 - DNA, 21 bp, Human
aaacttaatttccagtatggc

SEQ ID NO: 531 - DNA, 21 bp, Human
aatttccagtatggcagtatt

SEQ ID NO: 532 - DNA, 21 bp, Human
aagaggtaattggttcataca

SEQ ID NO: 533 - DNA, 21 bp, Human
aattggttcatacagattctg

SEQ ID NO: 534 - DNA, 21 bp, Human
aaatttcagtctcaggtattc

SEQ ID NO: 535 - DNA, 21 bp, Human
aaccacagaaaatagactaag

SEQ ID NO: 536 - DNA, 21 bp, Human
aagagccatcaaactgtatta

SEQ ID NO: 537 - DNA, 21 bp, Human
aagcaggggaatatatataca

SEQ ID NO: 538 - DNA, 21 bp, Human
aatcccaacactttttcatgt

SEQ ID NO: 539 - DNA, 21 bp, Human
aacactttttcatgttcttcc

SEQ ID NO: 540 - DNA, 21 bp, Human
aatgtgcaggttagttacata

SEQ ID NO: 541 - DNA, 21 bp, Human
aatatgcggtgtttggttttt

SEQ ID NO: 542 - DNA, 21 bp, Human
aactcatcactttttatggct

SEQ ID NO: 543 - DNA, 21 bp, Human
aaaaaacaaacaaccccatca

SEQ ID NO: 544 - DNA, 21 bp, Human
aaaacaaacaaccccatcaaa

SEQ ID NO: 545 - DNA, 21 bp, Human
aacaaacaaccccatcaaaaa

SEQ ID NO: 546 - DNA, 21 bp, Human
aacagacacttctcaaaagaa

SEQ ID NO: 547 - DNA, 21 bp, Human
aaaagaagacatttatgcagc

SEQ ID NO: 548 - DNA, 21 bp, Human
aagacatttatgcagccaaaa

SEQ ID NO: 549 - DNA, 21 bp, Human
aacacatgaaaaaatgctcac

SEQ ID NO: 550 - DNA, 21 bp, Human
aaaaccacaatgagataccat

SEQ ID NO: 551 - DNA, 21 bp, Human
aatggcaaccattaaaaagtc

SEQ ID NO: 552 - DNA, 21 bp, Human
aacacttttacactgttggta

SEQ ID NO: 553 - DNA, 21 bp, Human
aactagaaataccatttgacc

SEQ ID NO: 554 - DNA, 21 bp, Human
aaaggactataaatcatgctg

SEQ ID NO: 555 - DNA, 21 bp, Human
aaatcatgctgctataaagac

SEQ ID NO: 556 - DNA, 21 bp, Human
aaagtctttctttttatgggg

SEQ ID NO: 557 - DNA, 21 bp, Human
aaaattcttgttccagccttt

SEQ ID NO: 558 - DNA, 21 bp, Human
aagaatgttcctatgctgaaa

SEQ ID NO: 559 - DNA, 21 bp, Human
aatgttcctatgctgaaacta

SEQ ID NO: 560 - DNA, 21 bp, Human
aacaggtctgtatccatattt

SEQ ID NO: 561 - DNA, 21 bp, Human
aagccatttctttatttgtcc

SEQ ID NO: 562 - DNA, 21 bp, Human
aaacttcaagacatagtttgc

SEQ ID NO: 563 - DNA, 21 bp, Human
aagacatagtttgctcattct

SEQ ID NO: 564 - DNA, 21 bp, Human
aattagccaaaattctaccag

SEQ ID NO: 565 - DNA, 21 bp, Human
aattctaccagcaaactatga

SEQ ID NO: 566 - DNA, 21 bp, Human
aactttaatgagatagccctt

SEQ ID NO: 567 - DNA, 21 bp, Human
aatgagatagcccttgataat

SEQ ID NO: 568 - DNA, 21 bp, Human
aacaacataaaaccctcattc

SEQ ID NO: 569 - DNA, 21 bp, Human
aaatggaaatgcagatgttga

SEQ ID NO: 570 - DNA, 21 bp, Human
aataaacactattctacggga

SEQ ID NO: 571 - DNA, 21 bp, Human
aatagtctaatcacagacact

SEQ ID NO: 572 - DNA, 21 bp, Human
aatcacagacactcaatattc

SEQ ID NO: 573 - DNA, 21 bp, Human
aacctctatgataatctgcaa

SEQ ID NO: 574 - DNA, 21 bp, Human
aaaggaaaaagctgtgatgaa

SEQ ID NO: 575 - DNA, 21 bp, Human
aaagctgtgatgaatatcact

SEQ ID NO: 576 - DNA, 21 bp, Human
aatcctatcagatcacatttg

SEQ ID NO: 577 - DNA, 21 bp, Human
aatattgctgtgaaactgcat

SEQ ID NO: 578 - DNA, 21 bp, Human
aatagaaaataagtggtgctc

SEQ ID NO: 579 - DNA, 21 bp, Human
aaaataagtggtgctcaatga

SEQ ID NO: 580 - DNA, 21 bp, Human
aagaggagatcaaataacatg

SEQ ID NO: 581 - DNA, 21 bp, Human
aataaggtattggtgacacaa

SEQ ID NO: 582 - DNA, 21 bp, Human
aaggtattggtgacacaaata

SEQ ID NO: 583 - DNA, 21 bp, Human
aaatatcatgaacagtgtagc

SEQ ID NO: 584 - DNA, 21 bp, Human
aaaagccaaggataatctttc

SEQ ID NO: 585 - DNA, 21 bp, Human
aaatgtctcctgtatactcat

SEQ ID NO: 586 - DNA, 21 bp, Human
aaccctcaatacacatacata

SEQ ID NO: 587 - DNA, 21 bp, Human
aaacttacatttcctaaaggc

SEQ ID NO: 588 - DNA, 21 bp, Human
aacacagagtatgatgagaaa

SEQ ID NO: 589 - DNA, 21 bp, Human
aagagatttctctttccgttt

SEQ ID NO: 590 - DNA, 21 bp, Human
aaactgtgtaattctgtaagg

SEQ ID NO: 591 - DNA, 21 bp, Human
aaggtaggtactattactatc

SEQ ID NO: 592 - DNA, 21 bp, Human
aaactttagagcttgttccta

SEQ ID NO: 593 - DNA, 21 bp, Human
aatattgttctccccattatg

SEQ ID NO: 594 - DNA, 21 bp, Human
aagaaaatgtcagctcactta

SEQ ID NO: 595 - DNA, 21 bp, Human
aaaatgtcagctcacttaaca

SEQ ID NO: 596 - DNA, 21 bp, Human
aagacatgaaggtaagtttga

SEQ ID NO: 597 - DNA, 21 bp, Human
aatttgtttgagtccacctta

SEQ ID NO: 598 - DNA, 21 bp, Human
aaaagagtgtagattatgctc

SEQ ID NO: 599 - DNA, 21 bp, Human
aaatctcatgttgaaatgtgg

SEQ ID NO: 600 - DNA, 21 bp, Human
aaaagaaagtaaatgcagctg

SEQ ID NO: 601 - DNA, 21 bp, Human
aagaaagtaaatgcagctgat

SEQ ID NO: 602 - DNA, 21 bp, Human
aaagtaaatgcagctgatatc

SEQ ID NO: 603 - DNA, 21 bp, Human
aagaaagggaacttatagaac

SEQ ID NO: 604 - DNA, 21 bp, Human
aaagggaacttatagaacaga

SEQ ID NO: 605 - DNA, 21 bp, Human
aacagaaagttggtttagatc

SEQ ID NO: 606 - DNA, 21 bp, Human
aaagttggtttagatcagtga

SEQ ID NO: 607 - DNA, 21 bp, Human
aatcacaaagtgtagactcaa

SEQ ID NO: 608 - DNA, 21 bp, Human
aatcatctatctatctggtca

SEQ ID NO: 609 - DNA, 21 bp, Human
aattcctttacatagcaactg

SEQ ID NO: 610 - DNA, 21 bp, Human
aactacatagagttggtagta

SEQ ID NO: 611 - DNA, 21 bp, Human
aaaatagataggacatgttgc

SEQ ID NO: 612 - DNA, 21 bp, Human
aatagataggacatgttgcat

SEQ ID NO: 613 - DNA, 21 bp, Human
aattttatatggacctcacct

SEQ ID NO: 614 - DNA, 21 bp, Human
aaatatagattaaggctccca

SEQ ID NO: 615 - DNA, 21 bp, Human
aacatcaaataacaggtccaa

SEQ ID NO: 616 - DNA, 21 bp, Human
aatcagaagcaagaactgaaa

SEQ ID NO: 617 - DNA, 21 bp, Human
aagaactgaaatttagacacg

SEQ ID NO: 618 - DNA, 21 bp, Human
aatggatatgaaacttgaagg

SEQ ID NO: 619 - DNA, 21 bp, Human
aagataaaaaacctgtggcat

SEQ ID NO: 620 - DNA, 21 bp, Human
aaattagggtatggctttttc

SEQ ID NO: 621 - DNA, 21 bp, Human
aaatccaaagtttcagaaagc

SEQ ID NO: 622 - DNA, 21 bp, Human
aactccaaagcttatactctt

SEQ ID NO: 623 - DNA, 21 bp, Human
aaagcttatactcttcgaact

SEQ ID NO: 624 - DNA, 21 bp, Human
aatttgattgtaggttcactg

SEQ ID NO: 625 - DNA, 21 bp, Human
aaagtctcaacaaactggtaa

SEQ ID NO: 626 - DNA, 21 bp, Human
aaactacatcttccctatttc

SEQ ID NO: 627 - DNA, 21 bp, Human
aatcctatccaacaactttct

SEQ ID NO: 628 - DNA, 21 bp, Human
aacaactttctgcattctgtt

SEQ ID NO: 629 - DNA, 21 bp, Human
aactttctgcattctgttctt

SEQ ID NO: 630 - DNA, 21 bp, Human
aaacatactatgtaggttgac

SEQ ID NO: 631 - DNA, 21 bp, Human
aatataaccatgacctggata

SEQ ID NO: 632 - DNA, 21 bp, Human
aaaggttttggttagatcttc

SEQ ID NO: 633 - DNA, 21 bp, Human
aatagacttaattcagcagtc

SEQ ID NO: 634 - DNA, 21 bp, Human
aagagcaaggtatgatagaat

SEQ ID NO: 635 - DNA, 21 bp, Human
aagcaatctttgcaaatgaca

SEQ ID NO: 636 - DNA, 21 bp, Human
aattagtaaaggaatcctgga

SEQ ID NO: 637 - DNA, 21 bp, Human
aagtaaacaaaagctaggaag

SEQ ID NO: 638 - DNA, 21 bp, Human
aagtaatggtttaacttctgc

SEQ ID NO: 639 - DNA, 21 bp, Human
aaaatgtttccaatttcgctg

SEQ ID NO: 640 - DNA, 21 bp, Human
aactgatcagcaggaaattaa

SEQ ID NO: 641 - DNA, 21 bp, Human
aaaagaatttggcagttgcta

SEQ ID NO: 642 - DNA, 21 bp, Human
aacaagttacttaacctcact

SEQ ID NO: 643 - DNA, 21 bp, Human
aagttacttaacctcactaag

SEQ ID NO: 644 - DNA, 21 bp, Human
aacctcactaagattcagttt

SEQ ID NO: 645 - DNA, 21 bp, Human
aacatttccttctgctttttg

SEQ ID NO: 646 - DNA, 21 bp, Human
aagcctatatttgaagcctta

SEQ ID NO: 647 - DNA, 21 bp, Human
aatgcgtcctgcccctcccac

SEQ ID NO: 648 - DNA, 21 bp, Human
aatattctttattcctggaca

SEQ ID NO: 649 - DNA, 21 bp, Human
aagaagatggctcctgccatg

SEQ ID NO: 650 - DNA, 21 bp, Human
aagatggctcctgccatggaa

SEQ ID NO: 651 - DNA, 21 bp, Human
aatcccctactctactgtgtg

SEQ ID NO: 652 - DNA, 21 bp, Human
aaacctaaggtctccttgaac

SEQ ID NO: 653 - DNA, 21 bp, Human
aaggtctccttgaaccctcca

SEQ ID NO: 654 - DNA, 21 bp, Human
aaccctccatggaatagaata

SEQ ID NO: 655 - DNA, 21 bp, Human
aaaggagagaatgtgactctt

SEQ ID NO: 656 - DNA, 21 bp, Human
aagtcagttccaccaaatggt

SEQ ID NO: 657 - DNA, 21 bp, Human
aaatggttccacaatggcagc

SEQ ID NO: 658 - DNA, 21 bp, Human
aatggcagcctttcagaagag

SEQ ID NO: 659 - DNA, 21 bp, Human
aatacaaatgtcagcaccaac

SEQ ID NO: 660 - DNA, 21 bp, Human
aaatgtcagcaccaacaagtt

SEQ ID NO: 661 - DNA, 21 bp, Human
aagttaatgagagtgaacctg

SEQ ID NO: 662 - DNA, 21 bp, Human
aatgagagtgaacctgtgtac

SEQ ID NO: 663 - DNA, 21 bp, Human
aacctgtgtacctggaagtct

SEQ ID NO: 664 - DNA, 21 bp, Human
aagtcttcagtgactggctgc

SEQ ID NO: 665 - DNA, 21 bp, Human
aactgggatgtgtacaaggtg

SEQ ID NO: 666 - DNA, 21 bp, Human
aaggatggtgaagctctcaag

SEQ ID NO: 667 - DNA, 21 bp, Human
aagctctcaagtactggtatg

SEQ ID NO: 668 - DNA, 21 bp, Human
aagtactggtatgagaaccac

SEQ ID NO: 669 - DNA, 21 bp, Human
aaccacaacatctccattaca

SEQ ID NO: 670 - DNA, 21 bp, Human
aacatctccattacaaatgcc

SEQ ID NO: 671 - DNA, 21 bp, Human
aaatgccacagttgaagacag

SEQ ID NO: 672 - DNA, 21 bp, Human
aagacagtggaacctactact

SEQ ID NO: 673 - DNA, 21 bp, Human
aacctactactgtacgggcaa

SEQ ID NO: 674 - DNA, 21 bp, Human
aaagtgtggcagctggactat

SEQ ID NO: 675 - DNA, 22 bp, Human
baaguguggcagcuggacuaug

SEQ ID NO: 676 - DNA, 21 bp, Human
aataaaagctccgcgtgagaa

SEQ ID NO: 677 - DNA, 21 bp, Human
aaaagctccgcgtgagaagta

SEQ ID NO: 678 - DNA, 22 bp, Human
baaagcuccgcgugagaaguac

SEQ ID NO: 679 - DNA, 21 bp, Human
aagctccgcgtgagaagtact

SEQ ID NO: 680 - DNA, 21 bp, Human
aactcagcagcaggtcacatt

SEQ ID NO: 681 - DNA, 21 bp, Human
aagagaaccaggaaaggcttc

SEQ ID NO: 682 - DNA, 21 bp, Human
aaccaggaaaggcttcagact

SEQ ID NO: 683 - DNA, 21 bp, Human
aaaggcttcagacttctgaac

SEQ ID NO: 684 - DNA, 21 bp, Human
aacccacatcctaagccaaac

SEQ ID NO: 685 - DNA, 21 bp, Human
aagccaaaccccaaaaacaac

SEQ ID NO: 686 - DNA, 21 bp, Human
aattgtcaaacacagcttgca

SEQ ID NO: 687 - DNA, 21 bp, Human
aataggagagcaaatcttgct

SEQ ID NO: 688 - DNA, 21 bp, Human
aaatcttgctctcccacagga

SEQ ID NO: 689 - DNA, 21 bp, Human
aagtcttggaaatatctcccc

SEQ ID NO: 690 - DNA, 21 bp, Human
aaatatctccccaggaagtat

SEQ ID NO: 691 - DNA, 21 bp, Human
aagtatcttcaggcagactat

SEQ ID NO: 692 - DNA, 21 bp, Human
aagtcggcctcatccccacca

SEQ ID NO: 693 - DNA, 21 bp, Human
aaaagagcaggagttcctggg

SEQ ID NO: 694 - DNA, 21 bp, Human
aagagcaggagttcctggggg

SEQ ID NO: 695 - DNA, 21 bp, Human
aacagttgtctgctctgtact

SEQ ID NO: 696 - DNA, 21 bp, Human
aaagcaggttatccattctgg

SEQ ID NO: 697 - DNA, 21 bp, Human
aaatgcaacatatctggtgag

SEQ ID NO: 698 - DNA, 22 bp, Human
baatgcaacatatctggtgaga

SEQ ID NO: 699 - DNA, 21 bp, Human
aagcctgggagcaaacactgc

SEQ ID NO: 700 - DNA, 21 bp, Human
aaacactgccagcagcatagc

SEQ ID NO: 701 - DNA, 21 bp, Human
aacgggaattaccatcctgat

SEQ ID NO: 702 - DNA, 21 bp, Human
aacctgaagaagagcttggcc

SEQ ID NO: 703 - DNA, 21 bp, Human
aagaagagcttggcctatatc

SEQ ID NO: 704 - DNA, 21 bp, Human
aagagcttggcctatatccac

SEQ ID NO: 705 - DNA, 21 bp, Human
aagtgctttatggcttccttt

SEQ ID NO: 706 - DNA, 21 bp, Human
aatctgtggagctggggaaga

SEQ ID NO: 707 - DNA, 21 bp, Human
aactcaaaggaaacaaggttc

SEQ ID NO: 708 - DNA, 21 bp, Human
aagacccaggggaaatgtctc

SEQ ID NO: 709 - DNA, 21 bp, Human
aatcacgtgtccagaacactc

SEQ ID NO: 710 - DNA, 21 bp, Human
aacactctgattcacagccaa

SEQ ID NO: 711 - DNA, 21 bp, Human
aaggatccagaaggccaaggt

SEQ ID NO: 712 - DNA, 21 bp, Human
aaggccaaggttttgttaagg

SEQ ID NO: 713 - DNA, 21 bp, Human
aaggttttgttaaggggctac

SEQ ID NO: 714 - DNA, 21 bp, Human
aaggggctactggaaaaattt

SEQ ID NO: 715 - DNA, 21 bp, Human
aaatttctattctctccacag

SEQ ID NO: 716 - DNA, 21 bp, Human
aaagtcaacatgtagtaagca

SEQ ID NO: 717 - DNA, 21 bp, Human
aaaaaaagaaggctggctgaa

SEQ ID NO: 718 - DNA, 21 bp, Human
aaaaagaaggctggctgaaag

SEQ ID NO: 719 - DNA, 21 bp, Human
aaagaaggctggctgaaagtt

SEQ ID NO: 720 - DNA, 21 bp, Human
aaggctggctgaaagttgagt

SEQ ID NO: 721 - DNA, 21 bp, Human
aatagttacagttgggcatga

SEQ ID NO: 722 - DNA, 21 bp, Human
aaagatctgtataattgcctg

SEQ ID NO: 723 - DNA, 21 bp, Human
aattgcctgaatcaccagcac

SEQ ID NO: 724 - DNA, 21 bp, Human
aatcaccagcacattcactga

SEQ ID NO: 725 - DNA, 21 bp, Human
aagtaggaagtggggaacttt

SEQ ID NO: 726 - DNA, 21 bp, Human
aagtggggaacttttattaag

SEQ ID NO: 727 - DNA, 21 bp, Human
aagttactcgttgtctgggga

SEQ ID NO: 728 - DNA, 21 bp, Human
aaacagggaaattataagtgc

SEQ ID NO: 729 - DNA, 21 bp, Human
aagtgcagagattaacatttc

SEQ ID NO: 730 - DNA, 21 bp, Human
aagactaaattaagacctgag

SEQ ID NO: 731 - DNA, 21 bp, Human
aaattaagacctgagctgaaa

SEQ ID NO: 732 - DNA, 21 bp, Human
aagacctgagctgaaataaag

SEQ ID NO: 733 - DNA, 21 bp, Human
aaataaagtgacgtggaaatg

SEQ ID NO: 734 - DNA, 21 bp, Human
aaagtgacgtggaaatggaaa

SEQ ID NO: 735 - DNA, 21 bp, Human
aaagttagacaagcaactggt

SEQ ID NO: 736 - DNA, 21 bp, Human
aagcaactggttgactaatac

SEQ ID NO: 737 - DNA, 21 bp, Human
aatacattaagcgtttgagtc

SEQ ID NO: 738 - DNA, 21 bp, Human
aagcgtttgagtctaagatga

SEQ ID NO: 739 - DNA, 21 bp, Human
aagatgaaaggagaacactgg

SEQ ID NO: 740 - DNA, 21 bp, Human
aaaggagaacactggttatgt

SEQ ID NO: 741 - DNA, 21 bp, Human
aacactggttatgttgataga

SEQ ID NO: 742 - DNA, 21 bp, Human
aatgataaaaagggtcgggcg

SEQ ID NO: 743 - DNA, 21 bp, Human
aaaaagggtcgggcgcggagg

SEQ ID NO: 744 - DNA, 21 bp, Human
aatcccagccctttgggaggc

SEQ ID NO: 745 - DNA, 21 bp, Human
aagtcagtagtttgagaccag

SEQ ID NO: 746 - DNA, 21 bp, Human
aacatagtgaaaccccgtctc

SEQ ID NO: 747 - DNA, 21 bp, Human
aaaccccgtctctactaaaaa

SEQ ID NO: 748 - DNA, 21 bp, Human
aaaaaaaattagctgggtgtg

SEQ ID NO: 749 - DNA, 21 bp, Human
aaaaaattagctgggtgtggt

SEQ ID NO: 750 - DNA, 21 bp, Human
aaaattagctgggtgtggtgg

SEQ ID NO: 751 - DNA, 21 bp, Human
aattagctgggtgtggtggca

SEQ ID NO: 752 - DNA, 21 bp, Human
aatcgcttgaacctgggaggc

SEQ ID NO: 753 - DNA, 21 bp, Human
aacctgggaggcggaggttgc

SEQ ID NO: 754 - DNA, 21 bp, Human
aatgggagactccatctcaaa

SEQ ID NO: 755 - DNA, 21 bp, Human
aaatctgaaaagtggaggaag

SEQ ID NO: 756 - DNA, 21 bp, Human
aaaagtggaggaagagtacaa

SEQ ID NO: 757 - DNA, 21 bp, Human
aagtggaggaagagtacaaat

SEQ ID NO: 758 - DNA, 21 bp, Human
aagtctcattttttggctttg

SEQ ID NO: 759 - DNA, 21 bp, Human
aaagggaaatgcagccataga

SEQ ID NO: 760 - DNA, 21 bp, Human
aaatgcagccatagagggcct

SEQ ID NO: 761 - DNA, 21 bp, Human
aatacatgagttctggtaaag

SEQ ID NO: 762 - DNA, 21 bp, Human
aaaatttgatacacggtttgg

SEQ ID NO: 763 - DNA, 21 bp, Human
aatttgatacacggtttggtg

SEQ ID NO: 764 - DNA, 21 bp, Human
aaatgaagcaagttaacactc

SEQ ID NO: 765 - DNA, 21 bp, Human
aagcaagttaacactctaaga

SEQ ID NO: 766 - DNA, 21 bp, Human
aagtgaagctaagctaaactt

SEQ ID NO: 767 - DNA, 21 bp, Human
aagctaagctaaacttcacat

SEQ ID NO: 768 - DNA, 21 bp, Human
aagctaaacttcacatgccta

SEQ ID NO: 769 - DNA, 21 bp, Human
aattggagggaaaaactaagg

SEQ ID NO: 770 - DNA, 21 bp, Human
aaggataaaatctagcctaga

SEQ ID NO: 771 - DNA, 21 bp, Human
aaaatctagcctagaagatac

SEQ ID NO: 772 - DNA, 21 bp, Human
aatctagcctagaagatacaa

SEQ ID NO: 773 - DNA, 21 bp, Human
aaacatgcattgtgaaactgt

SEQ ID NO: 774 - DNA, 21 bp, Human
aaactgtagagagcaggtagc

SEQ ID NO: 775 - DNA, 21 bp, Human
aataagtatccatcagagaca

SEQ ID NO: 776 - DNA, 21 bp, Human
aagtatccatcagagacagta

SEQ ID NO: 777 - DNA, 21 bp, Human
aagagaaaagtccacagtgat

SEQ ID NO: 778 - DNA, 21 bp, Human
aaaagtccacagtgataagca

SEQ ID NO: 779 - DNA, 21 bp, Human
aagtccacagtgataagcaac

SEQ ID NO: 780 - DNA, 21 bp, Human
aagcaactccacctaaggcat

SEQ ID NO: 781 - DNA, 21 bp, Human
aactccacctaaggcatgaat

SEQ ID NO: 782 - DNA, 21 bp, Human
aaggcatgaatatgcggcaga

SEQ ID NO: 783 - DNA, 21 bp, Human
aatatgcggcagagaaaacag

SEQ ID NO: 784 - DNA, 21 bp, Human
aacagcaatagtgaatgaatg

SEQ ID NO: 785 - DNA, 21 bp, Human
aatgaatgcaaaaggtgctga

SEQ ID NO: 786 - DNA, 21 bp, Human
aatgcaaaaggtgctgagcaa

SEQ ID NO: 787 - DNA, 21 bp, Human
aaaaggtgctgagcaaattcc

SEQ ID NO: 788 - DNA, 21 bp, Human
aaggtgctgagcaaattccac

SEQ ID NO: 789 - DNA, 21 bp, Human
aaattccacacatgagtattg

SEQ ID NO: 790 - DNA, 21 bp, Human
aacatttgcaaagacctttag

SEQ ID NO: 791 - DNA, 21 bp, Human
aaagacctttagagaaagaga

SEQ ID NO: 792 - DNA, 21 bp, Human
aaagagaatgggagcatatgt

SEQ ID NO: 793 - DNA, 21 bp, Human
aatgggagcatatgtgcgaaa

SEQ ID NO: 794 - DNA, 21 bp, Human
aaggtagtgaagaaaagcaag

SEQ ID NO: 795 - DNA, 21 bp, Human
aagtataagagtcctacagat

SEQ ID NO: 796 - DNA, 21 bp, Human
aagagtcctacagatggactg

SEQ ID NO: 797 - DNA, 21 bp, Human
aaatcagtctgagagtattag

SEQ ID NO: 798 - DNA, 21 bp, Human
aagattaaaaggtgacaggta

SEQ ID NO: 799 - DNA, 21 bp, Human
aaaaggtgacaggtagatgtt

SEQ ID NO: 800 - DNA, 21 bp, Human
aaggtgacaggtagatgttaa

SEQ ID NO: 801 - DNA, 21 bp, Human
aatttccctagattgtgaaag

SEQ ID NO: 802 - DNA, 21 bp, Human
aaagtgatcacgacaatcaca

SEQ ID NO: 803 - DNA, 21 bp, Human
aattaagtgacttggtatgct

SEQ ID NO: 804 - DNA, 21 bp, Human
aagtgacttggtatgctttat

SEQ ID NO: 805 - DNA, 21 bp, Human
aattgtagggcctgaggtttt

SEQ ID NO: 806 - DNA, 21 bp, Human
aataaaaaccctaccctttca

SEQ ID NO: 807 - DNA, 21 bp, Human
aaaaaccctaccctttcactg

SEQ ID NO: 808 - DNA, 21 bp, Human
aaaccctaccctttcactgtg

SEQ ID NO: 809 - DNA, 21 bp, Human
aagctgcatctctactcttga

SEQ ID NO: 810 - DNA, 21 bp, Human
aatcacatcacttccatggca

SEQ ID NO: 811 - DNA, 21 bp, Human
aaccctggtttaccaggacct

SEQ ID NO: 812 - DNA, 21 bp, Human
aagatcattctgagagttgcc

SEQ ID NO: 813 - DNA, 21 bp, Human
aatgctggggttctcttcaca

SEQ ID NO: 814 - DNA, 21 bp, Human
aacttcaatgtcatcttctcc

SEQ ID NO: 815 - DNA, 21 bp, Human
aatgtcatcttctccatgaag

SEQ ID NO: 816 - DNA, 21 bp, Human
aagaccactgaatgaacacct

SEQ ID NO: 817 - DNA, 21 bp, Human
aatgaacaccttttcatccag

SEQ ID NO: 818 - DNA, 21 bp, Human
aacaccttttcatccagcctt

SEQ ID NO: 819 - DNA, 21 bp, Human
aatttcttgctccataactac

SEQ ID NO: 820 - DNA, 21 bp, Human
aactactctatcccacgatgc

SEQ ID NO: 821 - DNA, 21 bp, Human
aattattagtgtgcttgtgac

SEQ ID NO: 822 - DNA, 21 bp, Human
aagataatggggaattaaggc

SEQ ID NO: 823 - DNA, 21 bp, Human
aatggggaattaaggcatcaa

SEQ ID NO: 824 - DNA, 21 bp, Human
aacagagttatcgaggagggg

SEQ ID NO: 825 - DNA, 21 bp, Human
aatcaccaaaagccaaggagg

SEQ ID NO: 826 - DNA, 21 bp, Human
aagcctcacccagaccttctt

SEQ ID NO: 827 - DNA, 21 bp, Human
aaggcacagggaagccctctg

SEQ ID NO: 828 - DNA, 21 bp, Human
aaggggtttcctgcagggaca

SEQ ID NO: 829 - DNA, 21 bp, Human
aaagatccttttcacaaaccc

SEQ ID NO: 830 - DNA, 21 bp, Human
aaaccccacgtctccagaggg

SEQ ID NO: 831 - DNA, 21 bp, Human
aaggaagggcaggggccattc

SEQ ID NO: 832 - DNA, 21 bp, Human
aaagtgcccaagcagtggttc

SEQ ID NO: 833 - DNA, 21 bp, Human
aagcagtggttcccatcaatc

SEQ ID NO: 834 - DNA, 21 bp, Human
aatcacccctcagcaccccag

SEQ ID NO: 835 - DNA, 21 bp, Human
aaagcaacaatcaacatcgac

SEQ ID NO: 836 - DNA, 21 bp, Human
aacaatcaacatcgacccttt

SEQ ID NO: 837 - DNA, 21 bp, Human
aatcaacatcgaccctttgtg

SEQ ID NO: 838 - DNA, 21 bp, Human
aacatcgaccctttgtggcaa

SEQ ID NO: 839 - DNA, 21 bp, Human
aaaaccagaagaggccatgct

SEQ ID NO: 840 - DNA, 22 bp, Human
baaaccagaagaggccatgctg

SEQ ID NO: 841 - DNA, 21 bp, Human
aaccagaagaggccatgctgt

SEQ ID NO: 842 - DNA, 21 bp, Human
aagaggccatgctgtggtcag

SEQ ID NO: 843 - DNA, 21 bp, Human
aagaatgcagctgcacagtca

SEQ ID NO: 844 - DNA, 21 bp, Human
aatgcagctgcacagtcacca

SEQ ID NO: 845 - DNA, 21 bp, Human
aaagctgactccccagcccac

SEQ ID NO: 846 - DNA, 21 bp, Human
aagagctgagaggtgacccag

SEQ ID NO: 847 - DNA, 21 bp, Human
aaatgtgcagcctccctggct

SEQ ID NO: 848 - DNA, 21 bp, Human
aagggtcttccacaacatcct

SEQ ID NO: 849 - DNA, 21 bp, Human
aacatcctaaaatgggagaga

SEQ ID NO: 850 - DNA, 21 bp, Human
aaaatgggagagaacgcatga

SEQ ID NO: 851 - DNA, 21 bp, Human
aatgggagagaacgcatgata

SEQ ID NO: 852 - DNA, 21 bp, Human
aacgcatgataggcatgacaa

SEQ ID NO: 853 - DNA, 21 bp, Human
aaagggcagggttccctcata

SEQ ID NO: 854 - DNA, 21 bp, Human
aaagagcactcagagatcact

SEQ ID NO: 855 - DNA, 22 bp, Human
baagagcactcagagatcacta

SEQ ID NO: 856 - DNA, 21 bp, Human
aagaagaaaggtgagcaatag

SEQ ID NO: 857 - DNA, 21 bp, Human
aagaaaggtgagcaataggtt

SEQ ID NO: 858 - DNA, 21 bp, Human
aaaggtgagcaataggttctt

SEQ ID NO: 859 - DNA, 22 bp, Human
baaggtgagcaataggttctta

SEQ ID NO: 860 - DNA, 21 bp, Human
aataggttcttcatggaaagg

SEQ ID NO: 861 - DNA, 21 bp, Human
aaaggaaaaacccagtggccg

SEQ ID NO: 862 - DNA, 21 bp, Human
aaaaacccagtggccgataat

SEQ ID NO: 863 - DNA, 21 bp, Human
aaacccagtggccgataatat

SEQ ID NO: 864 - DNA, 21 bp, Human
aaaagtgaaaggaaggaggag

SEQ ID NO: 865 - DNA, 21 bp, Human
aagtgaaaggaaggaggaggg

SEQ ID NO: 866 - DNA, 21 bp, Human
aaaggaaggaggaggggatga

SEQ ID NO: 867 - DNA, 21 bp, Human
aaggaggaggggatgaagggg

SEQ ID NO: 868 - DNA, 21 bp, Human
aagggggtggaggtgcttctt

SEQ ID NO: 869 - DNA, 21 bp, Human
aagaccactttttagggccag

SEQ ID NO: 870 - DNA, 21 bp, Human
aaaatgccccaatgcaggaga

SEQ ID NO: 871 - DNA, 21 bp, Human
aatgccccaatgcaggagagt

SEQ ID NO: 872 - DNA, 21 bp, Human
aatgcaggagagtcctctcac

SEQ ID NO: 873 - DNA, 21 bp, Human
aaagcggaatgaagcaagcct

SEQ ID NO: 874 - DNA, 21 bp, Human
aatgaagcaagcctgcctcag

SEQ ID NO: 875 - DNA, 21 bp, Human
aagcaagcctgcctcagtctg

SEQ ID NO: 876 - DNA, 21 bp, Human
aagcctgcctcagtctgacag

SEQ ID NO: 877 - DNA, 21 bp, Human
aaaaccccaaagcgggcagcg

SEQ ID NO: 878 - DNA, 21 bp, Human
aaacaacaccagaggaggggc

SEQ ID NO: 879 - DNA, 21 bp, Human
aaaaacacacaggttcacaca

SEQ ID NO: 880 - DNA, 22 bp, Human
baaaacacacaggttcacacac

SEQ ID NO: 881 - DNA, 21 bp, Human
aaacacacaggttcacacaca

SEQ ID NO: 882 - DNA, 22 bp, Human
baacacacaggttcacacacag

SEQ ID NO: 883 - DNA, 21 bp, Human
aacctctcatacacatttgca

SEQ ID NO: 884 - DNA, 21 bp, Human
aatcatgcactcacacaggct

SEQ ID NO: 885 - DNA, 21 bp, Human
aacacacacacagtctcagga

SEQ ID NO: 886 - DNA, 21 bp, Human
aaaaacattccctccaatgcc

SEQ ID NO: 887 - DNA, 21 bp, Human
aaacattccctccaatgccac

SEQ ID NO: 888 - DNA, 21 bp, Human
aatgccacctccgtgactctg

SEQ ID NO: 889 - DNA, 21 bp, Human
aacgggacaactatgacctta

SEQ ID NO: 890 - DNA, 21 bp, Human
aactatgaccttaccagccac

SEQ ID NO: 891 - DNA, 21 bp, Human
aagcagatgttcacctgccgt

SEQ ID NO: 892 - DNA, 21 bp, Human
aagatcttacagtcgtcctgc

SEQ ID NO: 893 - DNA, 21 bp, Human
aacatcacctggctggaggac

SEQ ID NO: 894 - DNA, 21 bp, Human
aaagcgagctcaccctcagcc

SEQ ID NO: 895 - DNA, 21 bp, Human
aagcactggctgtcagaccgc

SEQ ID NO: 896 - DNA, 21 bp, Human
aaggtcacacctttgaggaca

SEQ ID NO: 897 - DNA, 21 bp, Human
aacccgagaggggtgagcgcc

SEQ ID NO: 898 - DNA, 21 bp, Human
aagccggcccagcccgttcga

SEQ ID NO: 899 - DNA, 21 bp, Human
aagtcgcccacgatcacctgt

SEQ ID NO: 900 - DNA, 21 bp, Human
aaggggaccgtgaacctgacc

SEQ ID NO: 901 - DNA, 21 bp, Human
aacctgacctggtcccgggcc

SEQ ID NO: 902 - DNA, 21 bp, Human
aagcctgtgaaccactccacc

SEQ ID NO: 903 - DNA, 21 bp, Human
aaccactccaccagaaaggag

SEQ ID NO: 904 - DNA, 21 bp, Human
aaaggaggagaagcagcgcaa

SEQ ID NO: 905 - DNA, 21 bp, Human
aagcagcgcaatggcacgtta

SEQ ID NO: 906 - DNA, 21 bp, Human
aatggcacgttaaccgtcacg

SEQ ID NO: 907 - DNA, 21 bp, Human
aaccgtcacgtccaccctgcc

SEQ ID NO: 908 - DNA, 21 bp, Human
aagtctatgcgtttgcgacgc

SEQ ID NO: 909 - DNA, 21 bp, Human
aagcgcaccctcgcctgcctg

SEQ ID NO: 910 - DNA, 21 bp, Human
aacttcatgcctgaggacatc

SEQ ID NO: 911 - DNA, 21 bp, Human
aacgaggtgcagctcccggac

SEQ ID NO: 912 - DNA, 21 bp, Human
aagaccaagggctccggcttc

SEQ ID NO: 913 - DNA, 21 bp, Human
aagggctccggcttcttcgtc

SEQ ID NO: 914 - DNA, 21 bp, Human
aatgggagcagaaagatgagt

SEQ ID NO: 915 - DNA, 21 bp, Human
aaagatgagttcatctgccgt

SEQ ID NO: 916 - DNA, 2009 bp, Human
agctttctggggcaggccaggcctgaccttggctttggggcagggagggggctaaggtgaggcaggtggcgccagcaggtgcacacccaatgcccatgagcccagacactggacgctgaacctcgcggacagttaagaacccaggggcctctgcgcctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagcaggctcagcgctcctgcctggacgcatcccggctatgcagccccagtccagggcagcaaggcaggccccgtctgcctcttcacccggagcctctgcccgccccactcatgctcagggagagggtcttctggctttttcccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtcctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagccccgctccccgggctctcgcggtcgcacgaggatgcttggcacgtaccccctgtacatacttcccgggcgcccagcatggaaataaagcacccagcgctgccctgggcccctgcgagactgtgatggttctttccacgggtcaggccgagtctgaggcctgagtggcatgagggaggcagagcgggtc

SEQ ID NO: 917 - DNA, 1932 bp, Human
gcatcccctgcgcgaagctgggtgccccggagagtctgaccaccatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattcttcaaagtgacgcctcccccaggaagcgggccccagaaccagtacgggaacgtgctgtctctccccacacccacctcaggcctcggacgcgcccagcgttgggccgcaggcctggggggcactgccccgtcttatggaaacccgagcagcgacgtccaggcggatggagccttggggtcccggagcccgccgggagtgggcccagaagaagaggaaggggagggctatgaggaacctgacagtgaggaggactccgagttctatgagaacgactccaaccttgggcaggaccagctctcccaggatggcagcggctacgagaaccctgaggatgagcccctgggtcctgaggatgaagactccttctccaacgctgagtcttatgagaacgaggatgaagagctgacccagccggtcgccaggacaatggacttcctgagccctcatgggtcagcctgggaccccagccgggaagcaacctccctggggtcccagtcctatgaggatatgagaggaatcctgtatgcagccccccagctccgctccattcggggccagcctggacccaatcatgaggaagatgcagactcttatgagaacatggataatcccgatgggccagacccagcctggggaggagggggccgcatgggcacctggagcaccaggtgatcctcaggtggccagcctggatctcctcaagtccccaagattcacacctgactctgaaatctgaagacctcgagcagatgatgccaacctctggagcaatgttgcttaggatgtgtgcatgtgtgtaagtgtgtgtgtgtgtgtgtgtgtgtatacatgccagtgacacttccagtcccctttgtattccttaaataaactcaatgagctcttccaa

SEQ ID NO: 918 - DNA, 1653 bp, Human
agcgatttcatcttcaggcctggactacaccactcaccctcccagtgtgcttgagaaacaaactgcacccactgaactccgcagctagcatccaaatcagcccttgagatttgaggccttggagactcaggagttttgagagcaaaatgacaacacccagaaattcagtaaatgggactttcccggcagagccaatgaaaggccctattgctatgcaatctggtccaaaaccactcttcaggaggatgtcttcactggtgggccccacgcaaagcttcttcatgagggaatctaagactttgggggctgtccagattatgaatgggctcttccacattgccctggggggtcttctgatgatcccagcagggatctatgcacccatctgtgtgactgtgtggtaccctctctggggaggcattatgtatattatttccggatcactcctggcagcaacggagaaaaactccaggaagtgtttggtcaaaggaaaaatgataatgaattcattgagcctctttgctgccatttctggaatgattctttcaatcatggacatacttaatattaaaatttcccattttttaaaaatggagagtctgaattttattagagctcacacaccatatattaacatatacaactgtgaaccagctaatccctctgagaaaaactccccatctacccaatactgttacagcatacaatctctgttcttgggcattttgtcagtgatgctgatctttgccttcttccaggaacttgtaatagctggcatcgttgagaatgaatggaaaagaacgtgctccagacccaaatctaacatagttctcctgtcagcagaagaaaaaaaagaacagactattgaaataaaagaagaagtggttgggctaactgaaacatcttcccaaccaaagaatgaagaagacattgaaattattccaatccaagaagaggaagaagaagaaacagagacgaactttccagaacctccccaagatcaggaatcctcaccaatagaaaatgacagctctccttaagtgatttcttctgttttctgtttccttttttaaacattagtgttcatagcttccaagagacatgctgactttcatttcttgaggtactctgcacatacgcaccacatctctatctggcctttgcatggagtgaccatagctccttctctcttacattgaatgtagagaatgtagccattgtagcagcttgtgttgtcacgcttcttcttttgagcaactttcttacactgaagaaaggcagaatgagtgcttcagaatgtgatttcctactaacctgttccttggataggctttttagtatagtatttttttttgtcattttctccatcaacaaccagggagactgcacctgatggaaaagatatatgactgcttcatgacattcctaaactatcttttttttattccacatctacgtttttggtggagtcccttttgcatcattgttttaaggatgataaaaaaaaaataacaactagggacaatacagaacccattccatttatctttctacagggctgacattgtggcacattcttagagttaccacaccccatgagggaagctctaaatagccaacacccatctgttttttgtaaaaacagcatagctt

SEQ ID NO: 919 - DNA, 17196 bp, Human
  misc_feature (1)...(17196)
  misc_feature (1)...(17196): n = A,T,C or G
ttgcacaaactgcaattacttttgcaycaactgatggatcagaatctctgagagtgagccccagtttatttttgtgagcctcttccatgattctaatgctcagccactgatctcaccagcaaagctgtctcatcaggttacagcagcgtccagatcaaaaccacagttacttttgcacaaacctgataattggtcttggaagtgaatcagtaaatttaaaaagctccccaagtgattctaatatactgtcagggttaagagtcacagccttgcgtgaaattgggtgaaacctctctcaatatactttccttgtatcttttcttggattccaacttgagaagtagccttctgatgcctagctgctggttaggaaaaaagaggagaggatccatgtaggagacatgaaggtactggtcactggaaaagccatacaatttataaaaacggttgcttgggagttctgtttctgtgaatctctccactagaaaacaatccatggtccttatgctgatggaagtcactttctgagactgtgaagtcagggatgaggcttggaacatggggacacatatgttaaccttgagaaatcttatgtgggtgactagccagcctggatacacaatgccaactttcttgtctgtggccccaggttttctagtttctgttgcaaaccacctctgtttcattgaaatattctttcttttttattataaaatatttcaaatatacaggaaaatcagaagaacagtgaccaactataagcccaataccaaactttgccatgtctcatcatttagtacatgtgcttcagaatttttacaataaaataacagctataaagccccttgtccaatccaagttccctccttctacacttagaggtaagcactatccataaagtggcattcatcctttctatggatgttttatcattttaccaaaattagatacattttaaaacaaatgcatgttgttaaaaatgtatgcaaaacagtactgtttcatatagtcgtgatttttatataaatggcatcatccttcatgtatcctgcaatttgccatctttgcttcagtacgactttgagtgccatgctaatgcatgtagctttaattcaatcattttcactgatgcataaatcattaaaaattgtataaatgcttgagtttctcttgggtgtatatagaggtgtaattgttgggtcaatacatgtatacatctttaaatttactagatatgaataaattattttccaaagtagttctaattttatgaaactccttgataattctcaaaattctataacactttcccttctacaatatgtgaatgctggcagaaatcatttataaagcagcatatacagacacagaatattgagacaaggaaagggactaaaagtaagttagccattaaagaagatgttatgctaagagaaatagtgtactacattattgcatgatgaaactggaaactgccttccaactcagtggaagcatccttatttttcgaaacagacttcagcaaagagaagtggaagaattatgacaaaatttactgacaattgaatgaattttcaacagtttaacattaaactgaagtgagagagtgatacaatgtgagagtaaactataattaatccagtagaagagagacataaacaaacaacaacaataaaaccttttactgagccctgaagcagagcttctttgtgagattatgtgctttccctcttctcaaaagctaaggtagaggctgaagtccagtccaaattaggcttcccagcactgggctctttctgcctggaggaagaggcctgttctagaagttattgcaaggtgctacaaagtacatcaacaaattttcataactgctattaagctttcaaaaccaccagacaaagaaataggaatggattgtaaattccctagagaaaagctacatgtctaactacttcgagcaaatccatgttttattacttatggtacttcttatacaaaatgctttggagaagtaaaattgttaggatgtatgatggacaatatactggggaattattacctgtaatttaggatggctgaaaacattcactatgttaaaggagaaatagttactaatagaagtagtaaatatctttagatacaatggtttcttaatattttcgtgcattatttaactttgacatatgaactattttctaaaacttatgaaggaagaaagattttctttcctttgattggccatatactggcagtggaacttgaaatgactactaattaccactttaaattatacgttatctggcaagaatttatagattttctaggaggctaatagttggggaatgaagtcgattttacgtatctttgctccctgttaattggaattaaaagtgatattgaatgtgtgtaaatgtatttcagtatattgactaactattgtncttgtgattataagttacagaccctttnctgaacttgaccttttcatatatttgctatatttccanwangagctgttattgntcaacttccatctatatanggnatttttatataaanggcatacacacacacacacacacacacacacatatatatatatagagagagagagagagagagaaatckcaaattgttttgtgttaccaatgatctagaaagtttggaactgtttataaaataaaggttttattaggtatattgtcgctgttgtttttattgcttttaagtttagaggctttttttttcgtcttcaaatcatgtaagctaaagtgaatatactcatttggagcctttcccaatgtgttaaacttgctttaataagtttatcaacttttaatgtatattgttactcctgtaacagagaaattctatttttagcttggttagttgtgttgtcacatattaataagtaactttacagcttattaattnctgaagaaattcaaaagtacttcacttgattacactgtcgtatttccagtattgagaataaggctttctaatgcctcttaatattcaaattgaactccaacatatgaaaatcattttgcttcctatcagaataccaagtactcttgcgttttaaaaatgcttctgcaagttcttactttaggctgggcatgatggcatgagtttgaggccagtctgaacaacatagcaagatcccatttctaaaaaaattaatttaattaaaaaaataaaattttactttgtaaaattgtaaaatttcacttggtaaacttgtatgtatatttcaattaactaccaattatctgtatatcttggcagtgtcttttctcatccttcagtgaaaagtttttaaaaacttgattgctgataaattagggaagggaaagtaatttatttttaataaaatactattctaaatgtttcagatttagttttgtgtcatcactaaattatattaagttacatcactaaagtaaattagtttattaaagttctttttaaactgaagtctcggtaaagaagatcaactataggacttctccacatgtcagggagaagtagaaataaagatgatgagacatgaccaatcctgtgtcttcttgaagccaaagatatttgtttaaatgaagatttggataaactccaaggtaaacaaaagggctattaagaaaaggtagggaattctggaaggagtgtgaaaggacaccctcttccactacttactggcccctcactgttcttcagtatcacggccacctctggaggcagacataggatgacccgatgacagcaatccatctctgttcccaaagggtattctgcatcccttaaaactgtgaggggtcaagcctttgtttcagtacctcataaacacaaaatctgaaataagtaaaatacagtaaaatatgatatgcaaatccatgagttttgcaggaagacaggtccaaatggcaggaaaatctctttctgctttgactggaaggaatgaaaacagaaatggtgatgaaatgcctgcaaagcattaggcagtttggaaagtccctgcaaatgtccctgtcccccatcctccagaccaccccacaccattatcacgactagcaacatgaccaggtcacacagatgtttgaccatttttgtggactgctcattgattagcattatgtgacagaagtattccgaggcagcttactgcttactgtagaaggtgctggttttcagcgaagatagtcaagtggtttggaaggggaaagaattatcttagccggaatctagacactgattctactcattctgatactacattaattctgctttgcaaaggggaaagaatattccttcttatttgactcctaaatgaacttctttcactattttgagggcccaacatcccacacaccccaggccagtgcggaaaccccctaaaggaagcagttcctcttctttttggataactggccagatttttttccaggaaatctctaggattcattttgtttttttacatatgacttttatgttgtattcaagatacccaaactctaagtaatgtaagtttattagattgcaaaacattgcaactgttttaaaactccatattagtgtcatgacaggaaaccgtaactgatttttaaaatacttgaattgggaaaaattaagtctcatggagagtgattctcttctcttgcatccacctgcatggtctctaccatgcccctacacttgctatgtgttcttggcaataccccaggaggttcagacaatagctcatcaacactgagaaccacaacttgcagatgtggtagccccatttacacaatggcaattgctttgtgctggagcaaggatctcatccacaacctatggtctaagggtaacttgtgcataaatagaattctgctgtaaggcaggaggactagagatggtttagaatggcttgatatcaatctcctttataattcagcagaagctaggaagttccaaagctgatcatggaaacagatactatcttaggataaatcatagattgggaaacattttgctaaggacttctggttggtgcattctggttgctgttgacagcaaacataccctctcttcttgatctatctgcagtaatggccccttatacctttctaatattcatcattacttttatttttgtccctctgtttattcttgatcattctcactggtcatttgacaaattaattagattttttaagaacccaacttggctcctgtttttgatgtcactgattttttgttttaattttactatctcctttcttcggatttttttttctgacctcttaagacaatatatctaatatctctttctaaaacccgcattagctgtatcccataaattttaatgtttttttctcctatggctcagtttgaaaaacacactaatatatgttcgtttctattatgatttttttctgccctatgtataagttagacatgtctatcttaattttcaaacatacagtttttaaaaagtatttattgattattaatttctagctcaatggcgtagagatcagagaacatattccacatgatttcaatctttgaagtttttcgagacttgccctatgatctagcttgcaatcaatttttgtacattagctttctgtatctgaaaaaggatgtcttctgcatttatcaggcgcagtgttctataagttccctatgccttgcaagttaatcatgtataaattttcaatttatttgctaattttatcaacttattctatcaattatcaacagataaaatgtatctgtgaatttaaatttgcattgtaaatttgtctgtttcttcttgtagttctgttgaatttgtcttaaatgttttgaagatacactattatatgtagaaaaacttaaaattattatatcatctaagtgaactgttcttaaattttcttttgtcaacaattgatataattacatatatcttttactactgtttacataacatatctttttccattactgattttcaagttttctttatctttagggttttttaaatacagttttattaaaccataattcatataccatgcaattcatccatttaaagtatgcaattcaatgacttttagtatatttacaaatacatttagtcattactagagtccattttagaacattttcatcacctgaagaatactcttttagtgtcatcctctatccactccctattatccatcctccctttatactcccctccgctgctaagcaatcactgatctatttcctgtctctatagattttcctgttgcagactttcacgtgaatggaatcatataatgtgtggacttttgttattggcttcctccagttagcataatgttttcatgcctcatccaagttgtaacatatcagtacatcattccatcttatggttttttccgttatatggatgtacacactttgtttatccatcaatccgttgattgacatttgagtcatctctaccttttgactgttatgaataatgctgctatgaacattcatgtacaagtttttgtgtgaacatatgctttcatttctattgggtatttacctaggagaggaattgctgggtttaaccatttgagaaactaccagattgctttccaaagtacctgcaccatttgatattcctgccagcagtgtacaagggttccagtttcactacatcctcatcagcactttttattatctgattgtttaatttcagccatcctggtgagctttgatttctgtttctagtggcccgaaattctacaagaatcaaagctcaaattctcccacacccctaaaatgacctagtagatttccactttctcttttatgttgccctaataatttctcttatacattctgattatatatatttatagggatatctagatagatgatagatagatagatatatagatagataggcaggatggatggacagataggatagatagatcgatctaaacacacacacacacacacacatacacacacacacacacacacgtatttcagcacttagggttgttttcagtggaataattcatccagttaacctaggacataacattactgaaaatagaattccattcttttacccattttaattggtatatcgttcctatgggtatgtagcagttcttccatgttctacataacagtctcctttttagttatatgtattgcagatgacttatgtcttatgtaagtcatgtggttgtgctcttgtaataatatattttaacaaatgaagtttttagtgttaatatacttgaatccatcaaacttttcctttatcatttgcattttcatgtctattataagaaatatttccttaccccaaggtcataaaagtactctcatatataggttcttctacattttttaatgtcttacctttcatgtttaagccatcatccattgggaatagaattaatatgtatgaggtttggattgattttattattccattatatgtaatgaattgtccccaaattatttgttgaatagtctctctcatctttcaccagcaatcttcagcactacccatgccatgaagctactttctatatatgaggtctttctcttttttcttattaatctatttttctactcattcatcaataccacactatcctaaatattaagaatttaggtcttgatacctactagaacaagtatcaggaagtgtcacttttactgtttgtgtccctttgctatgatgtattttagaacgacattgtcaggttcctttaaaaatgttaaattttgacaaaaattgcattgactatacatttgatcagtttgtagagagctcatatattttttaatcaagttgtcctgtacatgaatgtgcttatttcttgatttatttaggtcacattcactttacctgaataaaatgttataattttctccataaagattcttcaactattctgttagatttattcctattacgcacttttagtaagcatcctcttcttaagatttaaaatgagctcagtttaaaacaaaatgcttctaatattttaccataaggaaagatatttgctgcaggtattttttgcaaatatctttatagggctaagggaactcttttctatttctctgtagtttaaagggcttttaaaaatcggttaaattttattaaatcttttttttttttttttttttgagatgcagtcttgctctgtcgcccaggctggagtgcagtggtgcaatctcggcacactgcaagctccgcctcccggcttcacgccattctcctgcctcagcctcccaagtagctgggactacaggagcccgccaccatgcccggctaagtttttgtatttttagtagagatggtatttcaccgtgttatcctggatggtctcgatctcctgacctcatgatccgcccgcctgggcctcccaaagtgctgggattacagccgtgagccactgcacccggccaaatcttttatgagtttatgtttgtggtagattttaatctgttgaaaactatctttgtaatcctagcactttgaggacatggcaggaggattgcttgaggccaggagtttgagaccagcctgggcaacaagggaaaccctgtctctacagaaatctttaaaaatagccatgcatggtggcatgtgcctgtagtcccagctactgtggtgaggcaagaggatcacttgagcccaggagttcgaggctgcagtgagctatgatagtgatactgcactccagcctgggcaacacagcaagatactgtctctaacaaaacaatacaaaaaaactgaaggatggttcctctgaagaagaatgtgtttccttcagttagaagccagaggaccacaacagcactcggcttaattcaagtatcttaagtcaaattgcctaccttgtgcaactccctgcatataaagcttagtcaattgtagtaaaaattaacacaggacaacctcctttgtgtacacctacaaatcactgctccaattttagttccatttacctttttttcaaaattgtccccattggcaccaacatgtatgtactctggattttgtttattataaacaactacaaaatccttttgatgtatgtctaatgtcttaagcatattacaggatgaaaactctgtgcagaatccccattttctcatgagatttgtgtatttattcattcaaaaaatttctatgaagtgtctaccatggatcaggcaatattcatggaactagggggctcgaacatagagaaacagacagacaaaaaaacaaaatatttccttcatggatcttacattgtaagagagacaaacacaaatgaggtcattgtaaaatatataatgtgttcaatggagctaagtagtaagagaagaaaaatagaccaaagaaggggatatgaatggaaagggaggtaaaagtgtaagaggatatctaggaaagatctcactgagaaggtatcttcgagtaaagactagaatgaaatgagggagctggccactggggctattcaggagaaaaacaatctggacagagcaaacaagaccctccgagggcaaaggaccccagtcagagtgcaaagaatactagagcagcaaaggccattgtggccagagggagaaaagaaggagaaaagggtcaaagatgaactcagagaagttaaaggaaagcagatcacattggactgaatggatcatagttatgacattgccttctactttgaatgagataggaaattattggagggtttggagcagaaggcagtgatttcttttttcctgacaaacatgtaaacaggatcactgcaagatactagacaccaacacactcatctacagaggagttgagtttttgtgttgctgagaagtgtttattaataaaaatgtatttgggaaatttatgagggcagacaagaataagcatgaaaagaatcatgaatctaactgtaaaataaatgaaacaatacacgtgcaagaagaaaacatggatgaattattgtgtaacctggagaatggtttttaaaaccatggctcctaatccagatgcaataaaagaaaagatttatcaacttgactgcattaaaacagacaatgcttacaaggcaaaacgatgccataaacaaagtcaaaagacaactgacaaactgggaggaaacatttgcaaaacacatccagaataacaatcctaatatatataaataagtctcaaacattgagagaggaaaaggcaaatatacatgataaaaatatattttataaatgttcagcgttactcatagggaatgcaaattaaagctacactgagataaaatgtctcatttattagactgactaaaatttaaaaccatgatgacagcacattagattaatgaggtgctctcacacagttttgtaggaatatatgtggtacaactcttattaaggaaatttggcaatatctaacaaacctacatatgtgtttaccatttgacccagcaatcccacttctaggaatttgaactgaagacatgcttctaacagcagaaaaataaatattcacaaagttactctttagagcattaaaaacaaccaaactgtccatacatagggagatggttgaataaactatggcatatccacacagtgaagtattgcatgcaactgtaaaaatgaatgaggaatatttgaatttgtaggaaatagtggattccagcatatttgttaagttaaaaaagaaatgtgcaaaagaatacctatagcatgcttttaaaatgtaataaaaaaggagaactaagaagacaaatgtgtagcagctcatttgagcaaaactaaggacaaaccagaaactactgaaattgagtacctatggtggagaggatagaggtgaggagagaagtgacatttcccttttatkgtgtctttttatatcaatctgacatttgggactatgttaatgttttatttaagcaaaaaaaaaatcaaaaagattgaggggaaactaaaatagaatacaaagagaaacaaattaacagtatttttgaatgaataacaatcacactgaagagggggaataatctgagtaactttggaacacaccaatgctgtttttatattttcgggctataaacaaaataaaactttaaaaacgagtagatttgatttccatcatagtatagaaatagcaattttaaacttctttctgtgtattctaggattaagcaaataagtaaatatattgtggatctaggtgtctcactgttgaagaagggagttacacatgcagaaagagggaaggacagaatgaactctatgctaacggactgaaattggaagaatcactgtgaattcatgacatatataaatatagatatgggaatggatatacatatatatatataatacatatccttgctctgtctgctgagagggtgcggaaacaatgatactccaataaaaatgaacacacttagcatccaattctggcatctaatgccattctctgccagaaagaaccagcattccttggagaaacagcaaattccagtgtcaggtctagaggtgagtctggagcatttttttgtgtgtgtgtgccagaaagtaaggaagtgctcaaagaacaataagaacaagtcaaaatgacataggaactattttgagggctcccactggcccgatcagggagaatttaagcatcaaaataaataatggtagccatgaattatgacccattgagtaaataaataaaattcatattgatatagtttatgctgatatagataaatgaataaataaatagagaagggaaaacttttatttacagtaaaatgccaacagatgaatatagaagtaattattaaattaggaaaaaacaccctttgacaaccatcatagtaaaaattgattcaagaaaggatcatcaatagacgctaaaatttgtgggtaaaaatttgatgaaaaaatgaggaatttacatagttttaaagtatctccctataaaatacttattaattacaaagggaaaaatactgagtctacactaagaaacatggcaggcaccatcttaactaagtgatcaaagttaccaccaccagtaataggacaaatagacaacatatgcctccccgtaaggataagacatcctttctggtattcctgtcaaataattagattcaataacctgaatctatcatgaagaaatgatagacaaatccaaattgtggggtgtgatcctgtctcctgacattaccatccacgtatactgccctcctacactgaaaagggctgatctgtacaatcaattggatattctggagttaacatattgtgatttctgaggacaggtcacaaaggacattatggcttcttctttcttgttctcagagacgctagctggcatgtcacgaggacactcaacacacaagcagcgagccctatggcaaaatccacgtggtgaagaatgaagcttccagccaaaggctggtaagtgagccatcttagatgtggattcgcctatcccagttaacccttcagatgactgcacctccagctggtgtcttaactgcaatctcctgagcgatcccaagccagaaccacctgaccgacccacagaaactgtgagatcataaatgtttattcttgctttacaccactaccttcagggtaatttgttattataatagataacggacttaaaagtcattctccaaaaataattgtggtgtattcgtcaaaaatgtaaaggtcatgcaataccaaaaatgctgaggacatttttccagaataaagaactgggggtggggggaaggcataatgatcaaacacaatacatgatcctgaaatgattctggaccaggaaaaacaaaggttggtttctattgttgttattgtttttaccataacacaggttattgagatgattgtcaaaatttaaatacagatggtagcttatagtcttgtgtcaatgttaagttcttatttttgatcattgcactctagttatggaacagaaagtccttagtgaaccatttagaataggaataaatgctagatggttccaaagtaataattatataattataattatataatcacatatatgtacatatatgtctatagagaaagaatagagaatggtaaagaaaatacagcaaaatgttaacatgcagagaatctgggtgacaagtatatgggaattctctgcagtactcttgaaacttttctgtagagattacttcaaaataaagaacagagcagaagtggctgctgttcctaaattccctaagtaaacttacactgctttttcaaactgggagatgaatccactaataggttttgaaatgagtttggtggatttctaataagaataggatagcctagcctagtctagcataacacagcatggcacagaggacgaacattgtagattactaagggtgagtactaacttgtgaaagttttgtttaagttctagtaagagaaacaagtgagtgtagtgggttgcgtggtcaaaaaagtggggaagccaccgcaacgaggggtgagtctgagccaaagagtggctcagagctccccgcccacctcgtgccgggccgtccctcccgcttgtcccaccctcaccggcgccgcgtcagcccccaggccgcctgcaggtgtgcgctcagaactagcacgtgtgccggacactatttaagggcccgcctctcctggctcacagctgcttgctgctccagccttgccctcccagagctgccggacgctcgcgggtctcggaacgcatcccgccgcgggggcttcggccgtggcatgggcgccgcgggcctgctcggggttttcttggctctcgtcgcaccgggggtcctcggtgagctgggagggggagcacggaggtggggacgcgtcccgggcagggaaagtttctgtgccgcgatgcaaagcagggggccaaaagcgagacggtgggggcagtgctcgacgcgtgtccgcctcccgctagctttgagggaccactgcaataaaccgcctggtcctgattgtccccactggcccctccgggagctgggacctccagaattggaggctgcgccacagaggggcgctgtctggtcggcccggtgtggctggcggcgtgcgggtgctcgcggtctcctgccgggctccccgcccccaggattctgcaggtgctcatcgctctctggccggcggtcagcgctaccgctcgccacgggaaacctacccactgcatgaggcaccagctagccacgtgaggctgtttctgtacacccgaaccgcgctcttgcccagcaaaagcggcagaacctcggctcccttccctaaataagagtaagacctggttcccaagcatgggtctcagattttctgaactctaatgtgatattggtagagatcctaaacattaaccatccgtagttatttttgagacatggattaaaactattgttttcttcttgttattaacctactgtaatggtgtggacgtccgcaaaggtctcattcaccgtttattatatctttttttcctccagattggcttgattttaaagaaagattataattaagattagcgtctcttggctacaggatgggtctaagtttctaattatctccacggtaccgtgcattcctagtttataagacgtttactgtgtgcagacctcaataactgggtgacttttggcaagtcatttagctccctgatcttcagtttcttcatctgtaaaatgaaactagtgattaactggccttccacctcactgagctgccctgagcacagatgaaactaagcaaagaaagttggatgttagtcgttactactaaatatcagtaataagcctcctgtacagacttgattttatggagccagcatttaagctaacaagctgggtgtccaattgtttctatggacatccagcttattgaggtgtctggaatacagcacatattagtttattacaactttttgctttcctgacattttactgtctcctgacccttaaacctttctccagaccattatcttcatctcttttctttttctgcatattaactccctttgaacaaatgtatattcacactttttctaagagcagagtacgtccctgacttctcacctatttctaaatttcaaagaggatattatcaactgaaaatcaaattcttcaccagtcctgccaattcagggccacagcagcagcatcatgtaggtcacttaactggagccccatgaaataccaggtctacctatcagccattcaagctactgtgcttctgttgtttgtccatttctcatttttgtaccagtattcccatcctaagcattataattacctgtgtaacaacctcatctcttttaagagattttggttcttatgattggaaaggttaaagagtgacctataggtcactttccaattatgaaaacaaaaaattaagaaatacatatattttcattatttcactccattgttcaaaaatctaaaaggcctccagtggaagaaaatctggtgtcttcagcctttctagccctttggaatggaatctcaccgaagctcacaccccctgatttctccagctgcctgtcttgtatgcgctgctcatggtcttccccacctgtctcccatggctcatgctttgcccactctccactgctaacttttactgctactgcatgctctccacacatcatctcttggccagttcctgcacttgagtgtgcttaataaactactaaagtacctttataaaatgtaggttcctggttccacctccccctccagaggttcataggtttggagttagcacaggaaagctgaggtttaacaatcactctagaagattctgcaacaagtatctatagggcacactgctataagcaaagtcaatggaagcaaatttcctttttagtgttttcctatttttccttttgtctatttgcataaacagtttcaaatttcctacacttctagattttactcaagccttcagtggccacctaactttatcctatgckagaaaacaaaacccatgaaacaagaatgctcccattttctaatcagctacaaacctaccaacttacaaacttacctccatctagaatcagattttgttctccactgcagaaaactgctgaggccttcccgtcacattagaataaaacccataccccttattctccagtctgaggccttctgcctctccctgaaattctctgtaccccactcactctctctgccccagccacactggccttctctttattctttgagcacaattgcactaggtattctctcttttgaaatgctcttcctacaaattattctttgcctgtctccttgacattcaaggctcagccctctgttgtgcttctctgactcttgtctgaagtatccatattccagggtactctccaatacaaaactgtgttctattttctacagaccacctatcaccatctaaaaccttcttgcttatttatgtacctgtttgttgtttgttccctcactggaatacaagttccatgagtgcaagaccttaatctgccttatttcctgatttgagttcctatcaatagcctgtaatttggaattcaataaatatgaattgaaatcttggtatgtttcctcctataacaatgaaagaagtatccattctgctgacaaattcagctccttttcctgtattcatgatcatgttccataccctcttgcctttgcaaggactttacttctgcagatatctcctaccttgcatcatcaatgtacctctttctgtatgatcatccccrccagcatttaagtataccccaggaaatcctttatggaccttctcatgacccactggaactccattttcctgtccctcttcttgttcttctcagcaaaacttttcattgactacttactcaaatttcgttatctccttctccacttcagacaggcttcttccccacagtctcctgaagctgtttcttgcaatgtcgctactgacccttatgctgataaatccaatggtcatttttctatgttaatctttctcagtctttcagcaaaaactgatgacttcctttaccttgaaaccattatcattctcatcttccaaaccaagacactcttacctgttttttccatacttccctgttgtctcttagtcttttttgttctatctctaaatattagagggtactggggctcagccctgagccncttcttgcttcatttaytactt

SEQ ID NO: 920 - DNA, 3260 bp, Human
ccatcccatagtgagggaagacacgcggaaacaggcttgcacccagacacgacaccatgcatctcctcggcccctggctcctgctcctggttctagaatacttggctttctctgactcaagtaaatgggtttttgagcaccctgaaaccctctacgcctgggagggggcctgcgtctggatcccctgcacctacagagccctagatggtgacctggaaagcttcatcctgttccacaatcctgagtataacaagaacacctcgaagtttgatgggacaagactctatgaaagcacaaaggatgggaaggttccttctgagcagaaaagggtgcaattcctgggagacaagaataagaactgcacactgagtatccacccggtgcacctcaatgacagtggtcagctggggctgaggatggagtccaagactgagaaatggatggaacgaatacacctcaatgtctctgaaaggccttttccacctcatatccagctccctccagaaattcaagagtcccaggaagtcactctgacctgcttgctgaatttctcctgctatgggtatccgatccaattgcagtggctcctagagggggttccaatgaggcaggctgctgtcacctcgacctccttgaccatcaagtctgtcttcacccggagcgagctcaagttctccccacagtggagtcaccatgggaagattgtgacctgccagcttcaggatgcagatgggaagttcctctccaatgacacggtgcagctgaacgtgaagcacaccccgaagttggagatcaaggtcactcccagtgatgccatagtgagggagggggactctgtgaccatgacctgcgaggtcagcagcagcaacccggagtacacgacggtatcctggctcaaggatgggacctcgctgaagaagcagaatacattcacgctaaacctgcgcgaagtgaccaaggaccagagtgggaagtactgctgtcaggtctccaatgacgtgggcccgggaaggtcggaagaagtgttcctgcaagtgcagtatgccccggaaccttccacggttcagatcctccactcaccggctgtggagggaagtcaagtcgagtttctttgcatgtcactggccaatcctcttccaacaaattacacgtggtaccacaatgggaaagaaatgcagggaaggacagaggagaaagtccacatcccaaagatcctcccctggcacgctgggacttattcctgtgtggcagaaaacattcttggtactggacagaggggcccgggagctgagctggatgtccagtatcctcccaagaaggtgaccacagtgattcaaaaccccatgccgattcgagaaggagacacagtgaccctttcctgtaactacaattccagtaaccccagtgttacccggtatgaatggaaaccccatggcgcctgggaggagccatcgcttggggtgctgaagatccaaaacgttggctgggacaacacaaccatcgcctgcgcacgttgtaatagttggtgctcgtgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagcccgggggaccaagtgatggaggggaagagtgcaaccctgacctgtgagagtgacgccaaccctcccgtctcccactacacctggtttgactggaataaccaaagcctcccccaccacagccagaagctgagattggagccggtgaaggtccagcactcgggtgcctactggtgccaggggaccaacagtgtgggcaagggccgttcgcctctcagcacccttactgtctactatagcccggagaccatcggcaggcgagtggctgtgggactcgggtcctgcctcgccatcctcatcctggcaatctgtgggctcaagctccagcgacgttggaagaggacacagagccagcaggggcttcaggagaattccagcggccagagcttctttgtgaggaataaaaaggttagaagggcccccctctctgaaggcccccactccctgggatgctacaatccaatgatggaagatggcattagctacaccaccctgcgctttcccgagatgaacataccacgaactggagatgcagagtcctcagagatgcagagacctccccggacctgcgatgacacggtcacttattcagcattgcacaagcgccaagtgggcgactatgagaacgtcattccagattttccagaagatgaggggattcattactcagagctgatccagtttggggtcggggagcggcctcaggcacaagaaaatgtggactatgtgatcctcaaacattgacactggatgggctgcagcagaggcactgggggcagcgggggccagggaagtccccgagtttccccagacaccgccacatggcttcctcctgcgtgcatgtgcgcacacacacacacacacgcacacacacacacacacactcactgcggagaaccttgtgcctggctcagagccagtctttttggtgagggtaaccccaaacctccaaaactcctgcccctgttctcttccactctccttgctacccagaaatcatctaaatacctgccctgacatgcacacctcccctgccccaccagcccactggccatctccacccggagctgctgtgtcctctggatctgctcgtcattttccttcccttctccatctctctggccctctacccctgatctgacatccccactcacgaatattatgcccagtttctgcctctgagggaaagcccagaaaaggacagaaacgaagtagaaaggggcccagtcctggcctggcttctcctttggaagtgaggcattgcacggggagacgtacgtatcagcggccccttgactctggggactccgggtttgagatggacacactggtgtggattaacctgccagggagacagagctcacaataaaaatggctcagatgccacttcaaagaaaaaaaaaa

SEQ ID NO: 921 - DNA, 1158 bp, Human
ggaattccgtggccaggatgctgagcctgctgctgctggcgctgcccgtcctggcgagccgcgcctacgcggcccctgccccagtccaggccctgcagcaagcgggtatcgtcgggggtcaggaggcccccaggagcaagtggccctggcaggtgagcctgagagtccgcgaccgatactggatgcacttctgcgggggctccctcatccacccccagtgggtgctgaccgcggcgcactgcctgggaccggacgtcaaggatctggccaccctcagggtgcaactgcgggagcagcacctctactaccaggaccagctgctgccagtcagcaggatcatcgtgcacccacagttctacatcatccagactggagcggatatcgccctgctggagctggaggagcccgtgaacatctccagccgcgtccacacggtcatgctgccccctgcctcggagaccttccccccggggatgccgtgctgggtcactggctggggcgatgtggacaatgatgagcccctcccaccgccatttcccctgaagcaggtgaaggtccccataatggaaaaccacatttgtgacgcaaaataccaccttggcgcctacacgggagacgacgtccgcatcatccgtgacgacatgctgtgtgccgggaacagccagagggactcctgcaagggcgactctggagggcccctggtgtgcaaggtgaatggcacctggctacaggcgggcgtggtcagctgggacgagggctgtgcccagcccaaccggcctggcatctacacccgtgtcacctactacttggactggatccaccactatgtccccaaaaagccgtgagtcaggcctgggtgtgccacctgggtcactggaggaccaacccctgctgtccaaaacaccactgcttcctacccaggtggcgactgccccccacaccttccctgccccgtcctgagtgccccttcctgtcctaagccccctgctctcttctgagccccttcccctgtcctgaggacccttccccatcctgagcccccttccctgtcctaagcctgacgcctgcactgggccctccggccctcccctgcccaggcagctggtggtgggcgctaatcctcctgagtgctggacctcattaaagtgcatggaaatc

SEQ ID NO: 922 - DNA, 1193 bp, Human
ataaatggggaggggagagcccactgggtagaaggaacagggagcggccaggatgctgaatctgctgctgctggcgctgcccgtcctggcgagccgcgcctacgcggcccctgccccaggccaggccctgcagcgagtgggcatcgtcgggggtcaggaggcccccaggagcaagtggccctggcaggtgagcctgagagtccacggcccatactggatgcacttctgcgggggctccctcatccacccccagtgggtgctgaccgcagcgcactgcgtgggaccggacgtcaaggatctggccgccctcagggtgcaactgcgggagcagcacctctactaccaggaccagctgctgccggtcagcaggatcatcgtgcacccacagttctacaccgcccagatcggagcggacatcgccctgctggagctggaggagccggtgaacgtctccagccacgtccacacggtcaccctgccccctgcctcagagaccttccccccggggatgccgtgctgggtcactggctggggcgatgtggacaatgatgagcgcctcccaccgccatttcctctgaagcaggtgaaggtccccataatggaaaaccacatttgtgacgcaaaataccaccttggcgcctacacgggagacgacgtccgcatcgtccgtgacgacatgctgtgtgccgggaacacccggagggactcatgccagggcgactccggagggcccctggtgtgcaaggtgaatggcacctggctgcaggcgggcgtggtcagctggggcgagggctgtgcccagcccaaccggcctggcatctacacccgtgtcacctactacttggactggatccaccactatgtccccaaaaagccgtgagtcaggcctgggttggccacctgggtcactggaggaccaacccctgctgtccaaaacaccactgcttcctacccaggtggcgactgccccccacaccttccctgccccgtcctgagtgccccttcctgtcctaagccccctgctctcttctgagccccttcccctgtcctgaggacccttccctatcctgagcccccttccctgtcctaagcctgacgcctgcaccgggccctccagccctcccctgcccagatagctggtggtgggcgctaatcctcctgagtgctggacctcattaaagtgcatggaaatc

SEQ ID NO: 923 - DNA, 1148 bp, Human
ggccaggatgctgaatctgctgctgctggcgctgcccgtcctggcgagccgcgcctacgcggcccctgccccaggccaggccctgcagcgagtgggcatcgttgggggtcaggaggcccccaggagcaagtggccctggcaggtgagcctgagagtccacggcccatactggatgcacttctgcgggggctccctcatccacccccagtgggtgctgaccgcagcgcactgcgtgggaccggacgtcaaggatctggccgccctcagggtgcaactgcgggagcagcacctctactaccaggaccagctgctgccggtcagcaggatcatcgtgcacccacagttctacaccgcccagatcggagcggacatcgccctgctggagctggaggagccggtgaaggtctccagccacgtccacacggtcaccctgccccctgcctcagagaccttccccccggggatgccgtgctgggtcactggctggggcgatgtggacaatgatgagcgcctcccaccgccatttcctctgaagcaggtgaaggtccccataatggaaaaccacatttgtgacgcaaaataccaccttggcgcctacacgggagacgacgtccgcatcgtccgtgacgacatgctgtgtgccgggaacacccggagggactcatgccagggcgactccggagggcccctggtgtgcaaggtgaatggcacctggctgcaggcgggcgtggtcagctggggcgagggctgtgcccagcccaaccggcctggcatctacacccgtgtcacctactacttggactggatccaccactatgtccccaaaaagccgtgagtcaggcctgggttggccacctgggtcactggaggaccaacccctgctgtccaaaacaccactgcttcctacccaggtggcgactgccccccacaccttccctgccccgtcctgagtgccccttcctgtcctaagccccctgctctcttctgagccccttcccctgtcctgaggacccttccccatcctgagcccccttccctgtcctaagcctgacgcctgcaccgggccctccggccctcccctgcccaggcagctggtggtgggcgctaatcctcctgagtgctggacctcattaaagtgcatggaaatc

SEQ ID NO: 924 - DNA, 784 bp, Human
agcatttgctcaggcagcctctctgggaagatgctgcttcttcctctccccctgctgctctttctcttgtgctccagagctgaagctggggagatcatcgggggcacagaatgcaagccacattcccgcccctacatggcctacctggaaattgtaacttccaacggtccctcaaaattttgtggtggtttccttataagacggaactttgtgctgacggctgctcattgtgcaggaaggtctataacagtcacccttggagcccataacataacagaggaagaagacacatggcagaagcttgaggttataaagcaattccgtcatccaaaatataacacttctactcttcaccacgatatcatgttactaaagttgaaggagaaagccagcctgaccctggctgtggggacactccccttcccatcacaattcaactttgtcccacctgggagaatgtgccgggtggctggctggggaagaacaggtgtgttgaagccgggctcagacactctgcaagaggtgaagctgagactcatggatccccaggcctgcagccacttcagagactttgaccacaatcttcagctgtgtgtgggcaatcccaggaagacaaaatctgcatttaagggagactctgggggccctcttctgtgtgctggggtggcccagggcatcgtatcctatggacggtcggatgcaaagccccctgctgtcttcacccgaatctcccattaccggccctggatcaaccagatcctgcaggcaaattaatcctggatcc

SEQ ID NO: 925 - DNA, 1266 bp, Mouse
tctatcaggaaccctcagctctaccccttgaagccctgtaaaggcactgcttccatgaccctgggctgcctggtaaaggactacttccctggtcctgtgactgtgacctggtattcagactccctgaacatgagcactgtgaacttccctgcccttggttctgaactcaaggtcaccaccagccaagtgaccagctggggcaagtcagccaagaacttcacatgccacgtgacacatcctccatcattcaacgaaagtaggactatcctagttcgacctgtcaacatcactgagcccaccttggagctactccattcatcctgcgaccccaatgctttccactccaccatccagctgtactgcttcatttatggccacatcctaaatgatgtctctgtcagctggctaatggacgatcgggagataactgatacacttgcacaaactgttctaatcaaggaggaaggcaaactagcctctacctgcagtaaactcaacatcactgagcagcaatggatgtctgaaagcaccttcacctgcaaggtcacctcccaaggcgtagactatttggcccacactcggagatgcccagatcatgagccacggggtgtgattacctacctgatcccacccagccccctggacctgtatcaaaacggtgctcccaagcttacctgtctggtggtggacctggaaagcgagaagaatgtcaatgtgacgtggaaccaagagaagaagactccagtctcagcatcccagtggtacactaagcaccaccataacgccacaaccagtatcacctccatcctgcctgtagttgccaaggactggattgaaggctacggctatcagtgcatagtggaccaccctgattttcccaagcccattgtgcgttccatcaccaagaccccaggccagcgctcagcccccgaggtatatgtgttcccaccaccagaggaggagagcgaggacaaacgcacactcacctgcttgatccagaacttcttccctgaggatatctctgtgcagtggctggaggatggcaaactgatctcaaacagccatcacagtaccacaacacccctgaaatccaatggctccaatcaaggcttcttcatcttcagtcgcctagaggtcgccaagacactctggacacagagaaaacagttcacctgccaagtgatccatgaggcacttcagaaacccaggaaactggagaaaacaatatccacgagccttggtaacacctccctccatccctcctag

SEQ ID NO: 926 - DNA, 19 bp, Mouse
aggcactgcttccatgacc

SEQ ID NO: 927 - DNA, 19 bp, Mouse
agcaccttcacctgcaagg

SEQ ID NO: 928 - DNA, 19 bp, Mouse
cgccacaaccagtatcacc

SEQ ID NO: 929 - DNA, 19 bp, Mouse
gacactctggacacagaga

SEQ ID NO: 930 - DNA, 19 bp, Mouse
gagctactccattcatcct

SEQ ID NO: 931 - DNA, 19 bp, Mouse
ggcaaactagcctctacct

SEQ ID NO: 932 - DNA, 63 bp, Mouse
gatccaggcactgcttccatgaccttcaagagaggtcatggaagcagtgcctttttttggaaa

SEQ ID NO: 933 - DNA, 1010 bp, Mouse
ggatagcccaattaatgaaaaaatggacacagaaaataggagcagagcagatcttgctctcccaaatccacaagaatcctccagtgcacctgacattgaactcttggaagcatctcctgccaaagcagccccaccaaagcagacatggcggacatttttgaagaaagagttggagttcctgggagcaacacaaattctggttggtttgatatgcctttgttttggaacaattgtctgctccgtactctatgtttcagactttgatgaagaagtgcttttactttataaactaggctatccattctggggtgcagtgctgtttgttttgtctggatttttgtcaattatctccgaaagaaaaaacacattgtatctggtgagaggcagcctgggagcaaacattgtcagtagcatcgctgcagggacggggatcgccatgctgatcctcaatctgaccaataacttcgcttatatgaacaactgcaagaatgtaaccgaagacgacggctgctttgtggcttctttcaccacagaactggtgttgatgatgctgtttctcaccatcctggccttttgcagtgctgtgttgttcactatctataggattggacaagagttagaaagtaaaaaggtcccagatgatcgtctttatgaagaattaaatgtgtattcaccaatttacagtgagttggaagacaaaggggaaacatcttctccagttgattcataagaatcaggggaccaggacaatctgattcaagtataatcttgaaagttgatctttttacaaaattctcgcaaaatttctgtttgttccacattctgtcagtttttcaattggattgttctgcagatgccactcttttagttatgctgtatctgatcttctaaatatctccctttttgcggatatcattcactccaattttcttgttttgtgtcacaatttcacatacatcttttctggaaagtcatcaaggaataagttggctttattgtatgtctacttt

SEQ ID NO: 934 - DNA, 19 bp, Mouse
ctcaatctgaccaataact

SEQ ID NO: 935 - DNA, 19 bp, Mouse
agtgcacctgacattgaac

SEQ ID NO: 936 - DNA, 19 bp, Mouse
aaagtcatcaaggaataag

SEQ ID NO: 937 - DNA, 19 bp, Mouse
aggacaatctgattcaagt

SEQ ID NO: 938 - DNA, 66 bp, Mouse
gatccgctcaatctgaccaataactttcaagagaagttattggtcagattgaggattttttggaaa

SEQ ID NO: 939 - DNA, 19 bp, Mouse
gtgctttgtggcttctttc

SEQ ID NO: 940 - DNA, 66 bp, Mouse
gtgctttgtggcttctttcattcaagagatgaaagaagccacaaagcagcttttttggaaaagctt

SEQ ID NO: 941 - DNA, 19 bp, Mouse
gtcccagatgatcgtcttt

SEQ ID NO: 942 - DNA, 3975 bp, Human
ccggaaacagcgggctggggcagccactgcttacactgaagagggaggacgggagaggagtgtgtgtgtgtgtgtgtgtgtgtgtgtgtatgtatgtgtgtgctttatcttatttttctttttggtggtggtggtggaaggggggaggtgctagcagggccagccttgaactcgctggacagagctacagacctatggggcctggaagtgcccgctgagaaagggagaagacagcagaggggttgccgaggcaacctccaagtcccagatcatgtctctgtggggtctggtctccaagatgcccccagaaaaagtgcagcggctctatgtcgactttccccaacacctgcggcatcttctgggtgactggctggagagccagccctgggagttcctggtcggctccgacgccttctgctgcaacttggctagtgccctactttcagacactgtccagcaccttcaggcctcggtgggagagcagggggaggggagcaccatcttgcaacacatcagcacccttgagagcatatatcagagggaccccctgaagctggtggccactttcagacaaatacttcaaggagagaaaaaagctgttatggaacagttccgccacttgccaatgcctttccactggaagcaggaagaactcaagtttaagacaggcttgcggaggctgcagcaccgagtaggggagatccaccttctccgagaagccctgcagaagggggctgaggctggccaagtgtctctgcacagcttgatagaaactcctgctaatgggactgggccaagtgaggccctggccatgctactgcaggagaccactggagagctagaggcagccaaagccctagtgctgaagaggatccagatttggaaacggcagcagcagctggcagggaatggcgcaccgtttgaggagagcctggccccactccaggagaggtgtgaaagcctggtggacatttattcccagctacagcaggaggtaggggcggctggtggggagcttgagcccaagacccgggcatcgctgactggccggctggatgaagtcctgagaaccctcgtcaccagttgcttcctggtggagaagcagcccccccaggtactgaagactcagaccaagttccaggctggagttcgattcctgttgggcttgaggttcctgggggccccagccaagcctccgctggtcagggccgacatggtgacagagaagcaggcgcgggagctgagtgtgcctcagggtcctggggctggagcagaaagcactggagaaatcatcaacaacactgtgcccttggagaacagcattcctgggaactgctgctctgccctgttcaagaacctgcttctcaagaagatcaagcggtgtgagcggaagggcactgagtctgtcacagaggagaagtgcgctgtgctcttctctgccagcttcacacttggccccggcaaactccccatccagctccaggccctgtctctgcccctggtggtcatcgtccatggcaaccaagacaacaatgccaaagccactatcctgtgggacaatgccttctctgagatggaccgcgtgccctttgtggtggctgagcgggtgccctgggagaagatgtgtgaaactctgaacctgaagttcatggctgaggtggggaccaaccgggggctgctcccagagcacttcctcttcctggcccagaagatcttcaatgacaacagcctcagtatggaggccttccagcaccgttctgtgtcctggtcgcagttcaacaaggagatcctgctgggccgtggcttcaccttttggcagtggtttgatggtgtcctggacctcaccaaacgctgtctccggagctactggtctgaccggctgatcattggcttcatcagcaaacagtacgttactagccttcttctcaatgagcccgacggaacctttctcctccgcttcagcgactcagagattgggggcatcaccattgcccatgtcatccggggccaggatggctctccacagatagagaacatccagccattctctgccaaagacctgtccattcgctcactgggggaccgaatccgggatcttgctcagctcaaaaatctctatcccaagaagcccaaggatgaggctttccggagccactacaagcctgaacagatgggtaaggatggcaggggttatgtcccagctaccatcaagatgaccgtggaaagggaccaaccacttcctaccccagagctccagatgcctaccatggtgccttcttatgaccttggaatggcccctgattcctccatgagcatgcagcttggcccagatatggtgccccaggtgtacccaccacactctcactccatccccccgtatcaaggcctctccccagaagaatcagtcaacgtgttgtcagccttccaggagcctcacctgcagatgccccccagcctgggccagatgagcctgccctttgaccagcctcacccccagggcctgctgccgtgccagcctcaggagcatgctgtgtccagccctgaccccctgctctgctcagatgtgaccatggtggaagacagctgcctgagccagccagtgacagcgtttcctcagggcacttggattggtgaagacatattccctcctctgctgcctcccactgaacaggacctcactaagcttctcctggaggggcaaggggagtcggggggagggtccttgggggcacagcccctcctgcagccctcccactatgggcaatctgggatctcaatgtcccacatggacctaagggccaaccccagttggtgatcccagctggagggagaacccaaagagacagctcttctactacccccacagacctgctctggacacttgctcatgccctgccaagcagcagatggggagggtgccctcctatccccacctactcctgggtcaggaggaaaagactaacaggagaatgcacagtgggtggagccaatccactccttcctttctatcattcccctgcccacctccttccagcactgactggaagggaagttcaggctctgagacacaccccaacatgcctgcacctgcagcgcgcacacgcacgcacacacacatacagagctctctgagggtgatggggctgagcaggaggggggctgggtaagagcacaggttagggcatggaaggcttctccgcccattctgacccagggcctaggacggataggcaggaacatacagacacatttacactagaggccagggatagaggatattgggtctcagccctaggggaatgggaagcagctcaagggaccctgggtgggagcataggaggggtctggacatgtggttactagtacaggttttgccctgattaaaaaatctcccaaagccccaaattcctgttagccaggtggaggcttctgatacgtgtatgagactatgcaaaagtacaagggctgagattcttcgtgtatagctgtgtgaacgtgtatgtacctaggatatgttaaatgtatagctggcaccttagttgcatgaccacatagaacatgtgtctatctgcttttgcctacgtgacaacacaaatttgggagggtgagacactgcacagaagacagcagcaagtgtgctggcctctctgacatatgctaacccccaaatactctgaatttggagtctgactgtgcccaagtgggtccaagtggctgtgacatctacgtatggctccacacctccaatgctgcctgggagccagggtgagagtctgggtccaggcctggccatgtggccctccagtgtatgagagggccctgcctgctgcatcttttctgttgccccatccaccgccagcttcccttcactcccctatcccattctccctctcaaggcaggggtcatagatcctaagccataaaataaattttattccaaaataacaaaataaataatctactgtacacaatctg

SEQ ID NO: 943 - DNA, 1000 bp, Human
aggtgtccgggagtaaaggcctctctggatcccttggtctcctccagctcctcccccagcaaaaactgcagaaccctccactagttatgttgatgactcagaagttgagcaagactgtgtgtgtgtgtgtgtgtgtgtgtgtgtgtgtgtgtgtgtggtgttgtgattgcaatgggctctgtttgtgagcctgcctgcacgtgtgtgtgtgtgtgtgtgtgtgtgtgtgtgtagtcttgtggtcagggaagttgtgcatgtgtgtgtttgtttcttggcgtgtctcagtgtttaccccagaaacatataggaacttggcagataggaacacagcagattcgtattcaaacttgccccttgtgaatctgcaggcagcagctccggcttgtgctggttcccaccacagtctcaggaggggtgccctgtgaggagagagcaaagaccagcttcagtccaagggactcctagagtcttccagaattctgagctgagggttcccctcccccactccctcccgtcagtggtcacgagaccgacctctaaggcgttccctgccggaagggagggggacctaggagttggctggcatcgagctccctggcggctttttagggtcctccactggagggagcgcagagtccagagggatttacttttcctgaggccctggggagcccagtcccttgtgggtccaaaccccagcccttggcagagtttgagtttgggagccaggcagttaggggtggcaaatctctgtttgatattgggtgactttctggagaaaagctgatgcttttgagggggacagagtaagtgggggtcagcctccccccaagcctggctccaaggcctggaccccagtcctgatcccccacgtgttcccccactcggcacaggaggcacacatattcaccccactttcttcctcttcctcctccagcccactttctcttctctgtgtcgtcagagctccagggagggacctgggtagaaggagaag

SEQ ID NO: 944 - DNA, 21 bp, Artificial Sequence
  Made in a Lab
agccactgcttacactgaaga

SEQ ID NO: 945 - DNA, 21 bp, Artificial Sequence
  Made in a Lab
gccactgcttacactgaagag

SEQ ID NO: 946 - DNA, 21 bp, Artificial Sequence
  Made in a Lab
gggagaggagtgtgtgtgtgt

SEQ ID NO: 947 - DNA, 21 bp, Artificial Sequence
  Made in a Lab
gtgtatgtatgtgtgtgcttt

SEQ ID NO: 948 - DNA, 19 bp, Artificial Sequence
  Made in a Lab
ggacagagctacagaccta

SEQ ID NO: 949 - DNA, 21 bp, Artificial Sequence
  Made in a Lab
cacccttgagagcatatatca

SEQ ID NO: 950 - DNA, 21 bp, Artificial Sequence
  Made in a Lab
gtggccactttcagacaaata

SEQ ID NO: 951 - DNA, 21 bp, Artificial Sequence
  Made in a Lab
ccactttcagacaaatacttc

SEQ ID NO: 952 - DNA, 21 bp, Artificial Sequence
  Made in a Lab
gcacagcttgatagaaactcc

SEQ ID NO: 953 - DNA, 19 bp, Artificial Sequence
  Made in a Lab
aaagcctggtggacattta

SEQ ID NO: 954 - DNA, 21 bp, Artificial Sequence
  Made in a Lab
gcagaaagcactggagaaatc

SEQ ID NO: 955 - DNA, 21 bp, Artificial Sequence
  Made in a Lab
ctggagaaatcatcaacaaca

SEQ ID NO: 956 - DNA, 25 bp, Artificial Sequence
  Made in a Lab
ggtggtcatcgtccatggcaaccaa

SEQ ID NO: 957 - DNA, 21 bp, Artificial Sequence
  Made in a Lab
cccagaagatcttcaatgaca

SEQ ID NO: 958 - DNA, 21 bp, Artificial Sequence
  Made in a Lab
tgggccgtggcttcacctttt

SEQ ID NO: 959 - DNA, 19 bp, Artificial Sequence
  Made in a Lab
gtacgttactagccttctt

SEQ ID NO: 960 - DNA, 19 bp, Artificial Sequence
  Made in a Lab
ggatggctctccacagata

SEQ ID NO: 961 - DNA, 21 bp, Artificial Sequence
  Made in a Lab
cactacaagcctgaacagatg

SEQ ID NO: 962 - DNA, 19 bp, Artificial Sequence
  Made in a Lab
ctaccatggtgccttctta

SEQ ID NO: 963 - DNA, 21 bp, Artificial Sequence
  Made in a Lab
gcacttggattggtgaagaca

SEQ ID NO: 964 - DNA, 25 bp, Artificial Sequence
  Made in a Lab
gcacaggttagggcatggaaggctt

SEQ ID NO: 965 - DNA, 21 bp, Artificial Sequence
  Made in a Lab
gaggccagggatagaggatat

SEQ ID NO: 966 - DNA, 21 bp, Artificial Sequence
  Made in a Lab
ggctgagattcttcgtgtata

SEQ ID NO: 967 - DNA, 21 bp, Artificial Sequence
  Made in a Lab
acgtgtatgtacctaggatat

SEQ ID NO: 968 - DNA, 25 bp, Artificial Sequence
  Made in a Lab
gcaagtgtgctggcctctctgacat

SEQ ID NO: 969 - DNA, 21 bp, Artificial Sequence
  Made in a Lab
gtggctgtgacatctacgtat

SEQ ID NO: 970 - DNA, 19 bp, Artificial Sequence
  Made in a Lab
atagatcctaagccataaa

SEQ ID NO: 971 - DNA, 25 bp, Artificial Sequence
  Made in a Lab
gagtaaaggcctctctggatccctt

SEQ ID NO: 972 - DNA, 21 bp, Artificial Sequence
  Made in a Lab
cagaaccctccactagttatg

SEQ ID NO: 973 - DNA, 21 bp, Artificial Sequence
  Made in a Lab
ctccactagttatgttgatga

SEQ ID NO: 974 - DNA, 21 bp, Artificial Sequence
  Made in a Lab
cttggcgtgtctcagtgttta

SEQ ID NO: 975 - DNA, 21 bp, Artificial Sequence
  Made in a Lab
catataggaacttggcagata

SEQ ID NO: 976 - DNA, 21 bp, Artificial Sequence
  Made in a Lab
acagcagattcgtattcaaac

SEQ ID NO: 977 - DNA, 25 bp, Artificial Sequence
  Made in a Lab
gggactcctagagtcttccagaatt

SEQ ID NO: 978 - DNA, 19 bp, Artificial Sequence
  Made in a Lab
tcacgagaccgacctctaa

SEQ ID NO: 979 - DNA, 21 bp, Artificial Sequence
  Made in a Lab
gtggcaaatctctgtttgata

SEQ ID NO: 980 - DNA, 21 bp, Artificial Sequence
  Made in a Lab
cggcacaggaggcacacatat